SEQUENCE LISTING (231 sequences)

SEQ ID NO: 1 - skipped

SEQ ID NO: 2 - protein, 19 aa, synthetic construct
MNFGLSLIFLALILKGVQC

SEQ ID NO: 3 - protein, 18 aa, synthetic construct
MRVLILLWLFTAFPGILS

SEQ ID NO: 4 - skipped

SEQ ID NO: 5 - protein, 5 aa, synthetic construct
NYVMS

SEQ ID NO: 6 - protein, 5 aa, synthetic construct
RFVMS

SEQ ID NO: 7 - protein, 5 aa, synthetic construct
SYVMS

SEQ ID NO: 8 - protein, 5 aa, synthetic construct
RNVMS

SEQ ID NO: 9 - protein, 5 aa, synthetic construct
RDVMS

SEQ ID NO: 10 - protein, 5 aa, synthetic construct
AYVMS

SEQ ID NO: 11 - protein, 6 aa, synthetic construct
SGYGWH

SEQ ID NO: 12 - protein, 18 aa, synthetic construct
  VARIANT 3..5: note = A C D E F G H I K L M N P Q R S T V W Y
  VARIANT 7..10: note = A C D E F G H I K L M N P Q R S T V W Y
  VARIANT 12..14: note = A C D E F G H I K L M N P Q R S T V W Y
  VARIANT 15: note = A C D E F G H I K L M N P Q R S T V W Y
TIXXXGXXXXYXXXXVKG

SEQ ID NO: 13 - protein, 17 aa, synthetic construct
TINSGGSYTYYPDTVKG

SEQ ID NO: 14 - protein, 17 aa, synthetic construct
TISSGGIHTYYLDSVKG

SEQ ID NO: 15 - protein, 18 aa, synthetic construct
TISYGGGHYTYYPDSVKG

SEQ ID NO: 16 - protein, 17 aa, synthetic construct
TISSGGVHTYYLDSVKG

SEQ ID NO: 17 - protein, 17 aa, synthetic construct
TINSGGRYAYYPDSVKG

SEQ ID NO: 18 - protein, 17 aa, synthetic construct
TINSVGRYTYYTDSVKG

SEQ ID NO: 19 - protein, 16 aa, synthetic construct
YINYSGHIELNPSLKD

SEQ ID NO: 20 - protein, 17 aa, synthetic construct
TINSGGRFTYYPDSVKG

SEQ ID NO: 21 - protein, 17 aa, synthetic construct
TISSNGIYIYYTDSVKG

SEQ ID NO: 22 - protein, 17 aa, synthetic construct
TINSGGRYTYYPDSVKG

SEQ ID NO: 23 - protein, 17 aa, synthetic construct
TISRGGSYIYYPDSVKG

SEQ ID NO: 24 - skipped

SEQ ID NO: 25 - protein, 6 aa, synthetic construct
GNFLDY

SEQ ID NO: 26 - protein, 6 aa, synthetic construct
GNALDY

SEQ ID NO: 27 - protein, 6 aa, synthetic construct
GNFLDY

SEQ ID NO: 28 - protein, 6 aa, synthetic construct
GNALNY

SEQ ID NO: 29 - protein, 6 aa, synthetic construct
GNNLDY

SEQ ID NO: 30 - protein, 6 aa, synthetic construct
GNNLDF

SEQ ID NO: 31 - protein, 12 aa, synthetic construct
GGGLYSSYGGDY

SEQ ID NO: 32 - protein, 6 aa, synthetic construct
GNNLDS

SEQ ID NO: 33 - protein, 20 aa, synthetic construct
MRFSAQLLGLLVLWIPGSTA

SEQ ID NO: 34 - protein, 17 aa, synthetic construct
  VARIANT 8..10: note = misc_feature - Xaa can be any naturally occurring amino acid
  VARIANT 17: note = misc_feature - Xaa can be any naturally occurring amino acid
RSSKSLLXXXGITYLYX

SEQ ID NO: 35 - protein, 16 aa, synthetic construct
RSSKSLLNSYGITYLY

SEQ ID NO: 36 - protein, 16 aa, synthetic construct
RSSKSLLHSNGITYLY

SEQ ID NO: 37 - protein, 16 aa, synthetic construct
RSSKSLLHYTGITYLY

SEQ ID NO: 38 - protein, 16 aa, synthetic construct
RSSKSLLQRNGITYLY

SEQ ID NO: 39 - protein, 11 aa, synthetic construct
LASQTIGTWLA

SEQ ID NO: 40 - protein, 16 aa, synthetic construct
RSSKSLLQRSGITYLY

SEQ ID NO: 41 - protein, 16 aa, synthetic construct
RSSKSLLHRNGITYLY

SEQ ID NO: 42 - protein, 16 aa, synthetic construct
RSSKSLLQINGITYLY

SEQ ID NO: 43 - protein, 7 aa, synthetic construct
  VARIANT 1..2: note = A C D E F G H I K L M N P Q R S T V W Y
XXSNLAS

SEQ ID NO: 44 - protein, 7 aa, synthetic construct
QMSNLAS

SEQ ID NO: 45 - protein, 7 aa, synthetic construct
RLSNLAS

SEQ ID NO: 46 - protein, 7 aa, synthetic construct
GAIRLAD

SEQ ID NO: 47 - protein, 7 aa, synthetic construct
RMSNLAS

SEQ ID NO: 48 - protein, 9 aa, synthetic construct
  VARIANT 4: note = A C D E F G H I K L M N P Q R S T V W Y
  VARIANT 9: note = A C D E F G H I K L M N P Q R S T V W Y
GQDXELPTX

SEQ ID NO: 49 - protein, 9 aa, synthetic construct
GQDLELPYT

SEQ ID NO: 50 - protein, 9 aa, synthetic construct
GQDLELPYS

SEQ ID NO: 51 - protein, 9 aa, synthetic construct
QQLYRSPYA

SEQ ID NO: 52 - protein, 9 aa, synthetic construct
GQDMELPYT

SEQ ID NO: 53 - protein, 336 aa, synthetic construct
  VARIANT 5: note = A C D E F G H I K L M N P Q R S T V W Y
  VARIANT 14..18: note = A C D E F G H I K L M N P Q R S T V W Y
  VARIANT 20: note = A C D E F G H I K L M N P Q R S T V W Y
  VARIANT 22: note = A C D E F G H I K L M N P Q R S T V W Y
  VARIANT 36: note = A C D E F G H I K L M N P Q R S T V W Y
  VARIANT 39: note = A C D E F G H I K L M N P Q R S T V W Y
  VARIANT 49: note = A C D E F G H I K L M N P Q R S T V W Y
  VARIANT 56: note = A C D E F G H I K L M N P Q R S T V W Y
  VARIANT 60: note = A C D E F G H I K L M N P Q R S T V W Y
  VARIANT 64: note = A C D E F G H I K L M N P Q R S T V W Y
  VARIANT 71..73: note = A C D E F G H I K L M N P Q R S T V W Y
  VARIANT 76: note = A C D E F G H I K L M N P Q R S T V W Y
  VARIANT 78: note = A C D E F G H I K L M N P Q R S T V W Y
  VARIANT 80: note = A C D E F G H I K L M N P Q R S T V W Y
  VARIANT 83: note = A C D E F G H I K L M N P Q R S T V W Y
  VARIANT 92: note = A C D E F G H I K L M N P Q R S T V W Y
  VARIANT 97..98: note = A C D E F G H I K L M N P Q R S T V W Y
  VARIANT 100..114: note = A C D E F G H I K L M N P Q R S T V W Y
  VARIANT 116..117: note = A C D E F G H I K L M N P Q R S T V W Y
  VARIANT 119..127: note = A C D E F G H I K L M N P Q R S T V W Y
  VARIANT 135..136: note = A C D E F G H I K L M N P Q R S T V W Y
  VARIANT 141: note = A C D E F G H I K L M N P Q R S T V W Y
  VARIANT 143: note = A C D E F G H I K L M N P Q R S T V W Y
  VARIANT 145: note = A C D E F G H I K L M N P Q R S T V W Y
  VARIANT 147: note = A C D E F G H I K L M N P Q R S T V W Y
  VARIANT 155: note = A C D E F G H I K L M N P Q R S T V W Y
  VARIANT 157: note = A C D E F G H I K L M N P Q R S T V W Y
  VARIANT 161: note = A C D E F G H I K L M N P Q R S T V W Y
  VARIANT 163: note = A C D E F G H I K L M N P Q R S T V W Y
  VARIANT 164: note = A C D E F G H I K L M N P Q R S T V W Y
  VARIANT 169..170: note = A C D E F G H I K L M N P Q R S T V W Y
  VARIANT 179..180: note = A C D E F G H I K L M N P Q R S T V W Y
  VARIANT 181..182: note = A C D E F G H I K L M N P Q R S T V W Y
  VARIANT 184..185: note = A C D E F G H I K L M N P Q R S T V W Y
  VARIANT 191: note = A C D E F G H I K L M N P Q R S T V W Y
  VARIANT 194: note = A C D E F G H I K L M N P Q R S T V W Y
  VARIANT 198: note = A C D E F G H I K L M N P Q R S T V W Y
  VARIANT 203..204: note = A C D E F G H I K L M N P Q R S T V W Y
  VARIANT 211: note = A C D E F G H I K L M N P Q R S T V W Y
  VARIANT 214: note = A C D E F G H I K L M N P Q R S T V W Y
  VARIANT 215..217: note = A C D E F G H I K L M N P Q R S T V W Y
  VARIANT 223: note = A C D E F G H I K L M N P Q R S T V W Y
  VARIANT 228..229: note = A C D E F G H I K L M N P Q R S T V W Y
  VARIANT 231..233: note = A C D E F G H I K L M N P Q R S T V W Y
  VARIANT 239..240: note = A C D E F G H I K L M N P Q R S T V W Y
  VARIANT 244
  VARIANT 246..253: note = A C D E F G H I K L M N P Q R S T V W Y
  VARIANT 257..260: note = A C D E F G H I K L M N P Q R S T V W Y
  VARIANT 262: note = A C D E F G H I K L M N P Q R S T V W Y
  VARIANT 264..265: note = A C D E F G H I K L M N P Q R S T V W Y
  VARIANT 267: note = A C D E F G H I K L M N P Q R S T V W Y
  VARIANT 269: note = A C D E F G H I K L M N P Q R S T V W Y
  VARIANT 271..272: note = A C D E F G H I K L M N P Q R S T V W Y
  VARIANT 275..278: note = A C D E F G H I K L M N P Q R S T V W Y
  VARIANT 282: note = A C D E F G H I K L M N P Q R S T V W Y
  VARIANT 284..287: note = A C D E F G H I K L M N P Q R S T V W Y
  VARIANT 289: note = A C D E F G H I K L M N P Q R S T V W Y
  VARIANT 295: note = A C D E F G H I K L M N P Q R S T V W Y
  VARIANT 300: note = A C D E F G H I K L M N P Q R S T V W Y
  VARIANT 301..305: note = A C D E F G H I K L M N P Q R S T V W Y
  VARIANT 307..313: note = A C D E F G H I K L M N P Q R S T V W Y
  VARIANT 315: note = A C D E F G H I K L M N P Q R S T V W Y
  VARIANT 317: note = A C D E F G H I K L M N P Q R S T V W Y
  VARIANT 322: note = A C D E F G H I K L M N P Q R S T V W Y
  VARIANT 324..327: note = A C D E F G H I K L M N P Q R S T V W Y
  VARIANT 329..330: note = A C D E F G H I K L M N P Q R S T V W Y
  VARIANT 332..334: note = A C D E F G H I K L M N P Q R S T V W Y
  VARIANT 219: note = A C D E F G H I K L M N P Q R S T V W Y
AKTTXPSVYPLAPXXXXXTXSXVTLGCLVKGYFPEXVTXTWNSGSLSSXVHTFPAXLQSXLYTXSSSVTVXXXTWXSXTXTCXVAHPASSTXVDKKXXPXXXXXXXXXXXXXXXCXXCXXXXXXXXXSVFIFPPXXKDVLXIXLXPXVTCVVVDXSXDDPXVXXSWFVXXVEVHTAQTXXXXEXXNSTIRXVSXLPIXHQDWXXGKEFKCXVNXXXXPXPIEXTISKXXGXXXAPQVYXXPPPXEXXXXXXXXLTCXXXXFXPXXIXVXWXXNGXXXXNYKXTXXXXDXDGSYFXYSKLXXXXXXWXXXXXXXCXVXHEGLXNXXXXKXXSXXXGK

SEQ ID NO: 54 - protein, 336 aa, synthetic construct
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 55 - protein, 335 aa, synthetic construct
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 56 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 57 - protein, 324 aa, synthetic construct; identical to SEQ ID NO: 56

SEQ ID NO: 58 - protein, 336 aa, synthetic construct; identical to SEQ ID NO: 54

SEQ ID NO: 59 - protein, 336 aa, synthetic construct
AKTTPPSVYPLAPGCGDTTGSSVTLGCLVKGYFPESVTVTWNSGSLSSSVHTFPALLQSGLYTMSSSVTVPSSTWLSQTVTCSVAHPASSTTVDKKLEPSGPISTIKPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPILDSDGSYFIYSKLNMKTSKWEKTDSFSCNVRHEGLKNNYLKKTISRSPGK

SEQ ID NO: 60 - protein, 336 aa, synthetic construct; identical to SEQ ID NO: 59

SEQ ID NO: 61 - protein, 327 aa, synthetic construct
AKTTAPSVYPLAPVCGDTTGSSVTLGCLVKGYFPEPVTLTWNSGSLSSDVHTFPALLQSGLYTLSSSVTVTTWPSQTITCNVAHPASSTKVDKKIEPRGSPTHKPCPPCPAPNLLGGPSVFIFPPKIKDVLMISLSPMVTCVVVDVSEDDPDVHVSWFVNNVEVHTAQTQTHREDYNSTIRVVSALPIQHQDWMSGKEFKCKVNNKALPAPIERTISKPKGPVRAPQVYVLPPPEEEMTKKQVTLTCMITDFMPEDIYVEWTNNGQTELNYKNTEPVLDSDGSYFMYSKLRVEKKNWVERNSYSCSVVHEGLHNHHTTKSFSRTPGK

SEQ ID NO: 62 - protein, 336 aa, synthetic construct; identical to SEQ ID NO: 59

SEQ ID NO: 63 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 64 - protein, 336 aa, synthetic construct; identical to SEQ ID NO: 59

SEQ ID NO: 65 - protein, 324 aa, synthetic construct; identical to SEQ ID NO: 63

SEQ ID NO: 66 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 67 - protein, 115 aa, synthetic construct
EVQLVESGGDLVQPGGSLILSCVASGFTFRNYVMSWIRQTPDKRLEWVATINSGGSYTYYPDTVKGRFTISRDNAKSTLYLQMSSLKSEDTAMYYCASGNFLDYWGQGTSVTVSS

SEQ ID NO: 68 - protein, 115 aa, synthetic construct
EVQLVESGGDLVKPGGSLKLSCAASGFTFSRFVMSWVRQTPDKRLEWVATISSGGIHTYYLDSVKGRVTIFRDNAKNTLYLQMSSLESEDTAMYYCAAGNALDYWGQGTSVTVSS

SEQ ID NO: 69 - protein, 115 aa, synthetic construct; identical to SEQ ID NO: 67

SEQ ID NO: 70 - protein, 116 aa, synthetic construct
EVHLVESGGDLVKPGGSLKLSCAASGFTFSSYVMSWVRQTPDKRLEWIATISYGGGHYTYYPDSVKGRFTISRDNAKNTLYLQMNSLKSDDTAMYYCSSGNALNYWGQGTSVTVSS

SEQ ID NO: 71 - protein, 115 aa, synthetic construct
EVQLVESGGDLVKPGGSLKLSCAASGFTFSRNVMSWVRQTPDKRLEWVATISSGGVHTYYLDSVKGRFTIFRDNAKNTLYLQMNSLQSEDTAMYYCAAGNALDYWGQGTSVTVSS

SEQ ID NO: 72 - protein, 115 aa, synthetic construct
EVQLVESGGDLVKPGGSLKLSCAASGFTFTNYVMSWVRQTPDKRLEWVATINSGGRYAYYPDSVKGRFTISRDNAKNTLYLQMSSLKSEDTAMYYCAGGNNLDYWGQGTTLTVSS

SEQ ID NO: 73 - protein, 115 aa, synthetic construct
EVQLVESGGDLVRPGGSLKLSCAASGFIFTNYVMSWVRQTPDKRLEWVATINSVGRYTYYTDSVKGRLTISRNNAKNTLYLQMNSLKSEDTAIYYCAGGNNLDFWGQGTTLTVSS

SEQ ID NO: 74 - protein, 121 aa, synthetic construct
AVQLQESGPALVKPSQSISITCTVTGYSITSGYGWHWIRQFPGNKLEWMGYINYSGHIELNPSLKDRISLSRDTSKNQFFLQLISVTTEDTATYYCVRGGGLYSSYGGDYWGQGTSLTVSS

SEQ ID NO: 75 - protein, 115 aa, synthetic construct
EVQLVESGGDLVRPGGSLKLSCASSGFIFSNYVMSWVRQTPDKRLEWVATINSGGRFTYYPDSVKGRFTISRDNAKNTLFLQMNSLKPEDTAIYYCAGGNNLDYWGQGTTLTVSS

SEQ ID NO: 76 - protein, 115 aa, synthetic construct
EVQLVESGGDLVKPGESLKLACAASGFTFSRDVMSWVRQTPDKRLEWVATISSNGIYIYYTDSVKGRFTISRDNAKNTLYLQLSSLRSEDTAVYYCSRGNNLDSWGQGTTLTVSS

SEQ ID NO: 77 - protein, 115 aa, synthetic construct
EVQLVESGGDLVKPGGSLKLSCAASGFTFSNYVMSWVRQTPDMRLEWVATINSGGRYTYYPDSVKGRFTISRDNAKNTLYLQMNSLKSEDTAIYYCAGGNNLDYWGQGTTLTVSS

SEQ ID NO: 78 - protein, 115 aa, synthetic construct
EVQLVESGGDLVKPGGSLKLSCAASGFSFSAYVMSWVRQTPDKRLEWVATISRGGSYIYYPDSVKGRFTISRDDARDTLYLQMTSLKSEDTAVYYCSGGNNLDYWGQGTALTVSS

SEQ ID NO: 79 - protein, 470 aa, synthetic construct
MNFGLSLIFLALILKGVQCEVQLVESGGDLVQPGGSLILSCVASGFTFRNYVMSWIRQTPDKRLEWVATINSGGSYTYYPDTVKGRFTISRDNAKSTLYLQMSSLKSEDTAMYYCASGNFLDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 80 - protein, 469 aa, synthetic construct
MNFGLSLIFLALILKGVQCEVQLVESGGDLVKPGGSLKLSCAASGFTFSRFVMSWVRQTPDKRLEWVATISSGGIHTYYLDSVKGRVTIFRDNAKNTLYLQMSSLESEDTAMYYCAAGNALDYWGQGTSVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 81 - protein, 458 aa, synthetic construct
MNFGLSLIFLALILKGVQCEVQLVESGGDLVQPGGSLILSCVASGFTFRNYVMSWIRQTPDKRLEWVATINSGGSYTYYPDTVKGRFTISRDNAKSTLYLQMSSLKSEDTAMYYCASGNFLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 82 - protein, 459 aa, synthetic construct
MNFGLSLISLALILKGVQCEVHLVESGGDLVKPGGSLKLSCAASGFTFSSYVMSWVRQTPDKRLEWIATISYGGGHYTYYPDSVKGRFTISRDNAKNTLYLQMNSLKSDDTAMYYCSSGNALNYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 83 - protein, 470 aa, synthetic construct
MNFGLSLIFLALILKGLQCEVQLVESGGDLVKPGGSLKLSCAASGFTFSRNVMSWVRQTPDKRLEWVATISSGGVHTYYLDSVKGRFTIFRDNAKNTLYLQMNSLQSEDTAMYYCAAGNALDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 84 - protein, 470 aa, synthetic construct
MNFGLSLIFLALILKGVQCEVQLVESGGDLVKPGGSLKLSCAASGFTFTNYVMSWVRQTPDKRLEWVATINSGGRYAYYPDSVKGRFTISRDNAKNTLYLQMSSLKSEDTAMYYCAGGNNLDYWGQGTTLTVSSAKTTPPSVYPLAPGCGDTTGSSVTLGCLVKGYFPESVTVTWNSGSLSSSVHTFPALLQSGLYTMSSSVTVPSSTWLSQTVTCSVAHPASSTTVDKKLEPSGPISTIKPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPILDSDGSYFIYSKLNMKTSKWEKTDSFSCNVRHEGLKNNYLKKTISRSPGK

SEQ ID NO: 85 - protein, 470 aa, synthetic construct
MNFGLSLIFLALILKGVQCEVQLVESGGDLVRPGGSLKLSCAASGFIFTNYVMSWVRQTPDKRLEWVATINSVGRYTYYTDSVKGRLTISRNNAKNTLYLQMNSLKSEDTAIYYCAGGNNLDFWGQGTTLTVSSAKTTPPSVYPLAPGCGDTTGSSVTLGCLVKGYFPESVTVTWNSGSLSSSVHTFPALLQSGLYTMSSSVTVPSSTWLSQTVTCSVAHPASSTTVDKKLEPSGPISTIKPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPILDSDGSYFIYSKLNMKTSKWEKTDSFSCNVRHEGLKNNYLKKTISRSPGK

SEQ ID NO: 86 - protein, 466 aa, synthetic construct
MRVLILLWLFTAFPGILSAVQLQESGPALVKPSQSISITCTVTGYSITSGYGWHWIRQFPGNKLEWMGYINYSGHIELNPSLKDRISLSRDTSKNQFFLQLISVTTEDTATYYCVRGGGLYSSYGGDYWGQGTSLTVSSAKTTAPSVYPLAPVCGDTTGSSVTLGCLVKGYFPEPVTLTWNSGSLSSDVHTFPALLQSGLYTLSSSVTVTTWPSQTITCNVAHPASSTKVDKKIEPRGSPTHKPCPPCPAPNLLGGPSVFIFPPKIKDVLMISLSPMVTCVVVDVSEDDPDVHVSWFVNNVEVHTAQTQTHREDYNSTIRVVSALPIQHQDWMSGKEFKCKVNNKALPAPIERTISKPKGPVRAPQVYVLPPPEEEMTKKQVTLTCMITDFMPEDIYVEWTNNGQTELNYKNTEPVLDSDGSYFMYSKLRVEKKNWVERNSYSCSVVHEGLHNHHTTKSFSRTPGK

SEQ ID NO: 87 - protein, 470 aa, synthetic construct
MNFGLSLIFLALILKGVQCEVQLVESGGDLVRPGGSLKLSCASSGFIFSNYVMSWVRQTPDKRLEWVATINSGGRFTYYPDSVKGRFTISRDNAKNTLFLQMNSLKPEDTAIYYCAGGNNLDYWGQGTTLTVSSAKTTPPSVYPLAPGCGDTTGSSVTLGCLVKGYFPESVTVTWNSGSLSSSVHTFPALLQSGLYTMSSSVTVPSSTWLSQTVTCSVAHPASSTTVDKKLEPSGPISTIKPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPILDSDGSYFIYSKLNMKTSKWEKTDSFSCNVRHEGLKNNYLKKTISRSPGK

SEQ ID NO: 88 - protein, 458 aa, synthetic construct
MNFGLSLIFLVLILKGVQCEVQLVESGGDLVKPGESLKLACAASGFTFSRDVMSWVRQTPDKRLEWVATISSNGIYIYYTDSVKGRFTISRDNAKNTLYLQLSSLRSEDTAVYYCSRGNNLDSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 89 - protein, 470 aa, synthetic construct
MNFGLSLIFLALILKGVQCEVQLVESGGDLVKPGGSLKLSCAASGFTFSNYVMSWVRQTPDMRLEWVATINSGGRYTYYPDSVKGRFTISRDNAKNTLYLQMNSLKSEDTAIYYCAGGNNLDYWGQGTTLTVSSAKTTPPSVYPLAPGCGDTTGSSVTLGCLVKGYFPESVTVTWNSGSLSSSVHTFPALLQSGLYTMSSSVTVPSSTWLSQTVTCSVAHPASSTTVDKKLEPSGPISTIKPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPILDSDGSYFIYSKLNMKTSKWEKTDSFSCNVRHEGLKNNYLKKTISRSPGK

SEQ ID NO: 90 - protein, 458 aa, synthetic construct
MNFGLSLIFLALILKGVQCEVQLVESGGDLVKPGGSLKLSCAASGFSFSAYVMSWVRQTPDKRLEWVATISRGGSYIYYPDSVKGRFTISRDDARDTLYLQMTSLKSEDTAVYYCSGGNNLDYWGQGTALTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 91 - protein, 112 aa, synthetic construct
DIVMTQAAFSNPVTLGTSASISCRSSKSLLNSYGITYLYWYLQKPGQSPQLLIYQMSNLASGVPDRFSSSGSGTDFTLRISRVEAEDVGVYYCGQDLELPYTFGSGTQLEIK

SEQ ID NO: 92 - protein, 112 aa, synthetic construct
DIVMTQAAFSNPVTLGTSGSVSCRSSKSLLHSNGITYLYWYLQKPGQSPQLLIYQMSNLASGVPDRFSSTGSGTDFTLRISRVEAEDVGVYYCGQDLELPYSFGSGTKLEIK

SEQ ID NO: 93 - protein, 112 aa, synthetic construct; identical to SEQ ID NO: 91

SEQ ID NO: 94 - protein, 112 aa, synthetic construct
DIVMTQAAFSNPVTLGTSASISCRSSKSLLHYTGITYLYWYLQKPGQSPQLLIYQMSNLASGVPDRFSSSGSGTDFTLRISRVEAEDVGVYYCGQDLELPYSFGSGTKLEIK

SEQ ID NO: 95 - protein, 112 aa, synthetic construct
DIMMTQAAFSNPVTLGTSGSISCRSSKSLLHSNGITYLYWYLQKPGQSPQLLIYQMSNLASGVPDRFSSSGSGTDFTLRISRVEAEDVGVYYCGQDLELPYSFGSGTKLEIK

SEQ ID NO: 96 - protein, 112 aa, synthetic construct
DIVMTQAAFSNPVTLGTSASISCRSSKSLLQRNGITYLYWYLQKPGQSPQLLIYRLSNLASGVPDRFSSSGSGTDFTLRISRVEAEDVGVYYCGQDLELPYTFGGGTKLEIK

SEQ ID NO: 97 - protein, 112 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCGQDLELPYSFGGGTKLEIK

SEQ ID NO: 98 - protein, 107 aa, synthetic construct
DIQMTQSPASQSASLGESVTITCLASQTIGTWLAWYHQKPGTSPQLLIYGAIRLADGVPSRFSGGGSGTEFSLKISSLQAEDFGSYYCQQLYRSPYAFGGGTNLEIK

SEQ ID NO: 99 - protein, 112 aa, synthetic construct
DIVMTQAEFSNPVTLGTSASISCRSSKSLLQRSGITYLYWYLQKPGQSPQLLIYRMSNLASGVPDRFSSSGSGTDFTLRISRVEAEDVGVYYCGQDMELPYTFGGGTKLEIK

SEQ ID NO: 100 - protein, 112 aa, synthetic construct
DIVMTQAAFSNPVTLGTSASISCRSSKSLLHRNGITYLYWYLQKPGQSPQLLIYQMSNLASGVSDRFSSSGSGTDFTLRISRVEAEDVGVYFCGQDLELPYSFGGGTKLEIK

SEQ ID NO: 101 - protein, 112 aa, synthetic construct
DIVMTQAAFSNPVTLGTSASISCRSSKSLLQRNGITYLYWYLQKPGQSPQLLIYRMSNLASGVPDRFSSSGSGTDFTLRISRVEAEDVGVYYCGQDLELPYTFGGGTKLEIK

SEQ ID NO: 102 - protein, 112 aa, synthetic construct
DIVMTQAAFSNPVTLGTSASISCRSSKSLLQINGITYLYWYLQKPGQSPQLLIYQMSNLASGVPDRFSSSGSGTDFTLRISRVEAEDVGVYYCGQDLELPYTFGGGTKLEIK

SEQ ID NO: 103 - protein, 239 aa, synthetic construct
MRFSAQLLGLLVLWIPGSTADIVMTQAAFSNPVTLGTSASISCRSSKSLLQINGITYLYWYLQKPGQSPQLLIYQMSNLASGVPDRFSSSGSGTDFTLRISRVEAEDVGVYYCGQDLELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 104 - protein, 239 aa, synthetic construct
MRFSAQLLGLLVLWIPGSTADIVMTQAAFSNPVTLGTSGSVSCRSSKSLLHSNGITYLYWYLQKPGQSPQLLIYQMSNLASGVPDRFSSTGSGTDFTLRISRVEAEDVGVYYCGQDLELPYSFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 105 - protein, 239 aa, synthetic construct
MRFSAQLLGLLVLWIPGSTADIVMTQAAFSNPVTLGTSASISCRSSKSLLNSYGITYLYWYLQKPGQSPQLLIYQMSNLASGVPDRFSSSGSGTDFTLRISRVEAEDVGVYYCGQDLELPYTFGSGTQLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 106 - protein, 239 aa, synthetic construct
MRFSAQLLGLLVLWIPGSTADIVMTQAAFSNPVTLGTSASISCRSSKSLLHYTGITYLYWYLQKPGQSPQLLIYQMSNLASGVPDRFSSSGSGTDFTLRISRVEAEDVGVYYCGQDLELPYSFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 107 - protein, 239 aa, synthetic construct
MRFSAQLLGLLVLWIPGSTADIMMTQAAFSNPVTLGTSGSISCRSSKSLLHSNGITYLYWYLQKPGQSPQLLIYQMSNLASGVPDRFSSSGSGTDFTLRISRVEAEDVGVYYCGQDLELPYSFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 108 - protein, 239 aa, synthetic construct
MRFSAQLLGLLVLWIPGSTADIVMTQAAFSNPVTLGTSASISCRSSKSLLQRNGITYLYWYLQKPGQSPQLLIYRLSNLASGVPDRFSSSGSGTDFTLRISRVEAEDVGVYYCGQDLELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - protein, 239 aa, synthetic construct
MRFSAQLLGLLVLWIPGSTADIVMTQAAFSNPVTLGTSASISCRSSKSLLHSNGITYLYWYLQKPGQSPQLLIYQMSNLASGVPDRFSSSGSGTDFTLRISRVEAEDVGVYYCGQDLELPYSFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 110 - protein, 219 aa, synthetic construct
DIVMTQAAFSNPVTLGTSASISCRSSKSLLQINGITYLYWYLQKPGQSPQLLIYQMSNLASGVPDRFSSSGSGTDFTLRISRVEAEDVGVYYCGQDLELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 111 - protein, 239 aa, synthetic construct
MRFSAQLLGLLVLWIPGSTADIVMTQAEFSNPVTLGTSASISCRSSKSLLQRSGITYLYWYLQKPGQSPQLLIYRMSNLASGVPDRFSSSGSGTDFTLRISRVEAEDVGVYYCGQDMELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 112 - protein, 239 aa, synthetic construct
MRFSAQLLGLLVLWIPGSTADIVMTQAAFSNPVTLGTSASISCRSSKSLLHRNGITYLYWYLQKPGQSPQLLIYQMSNLASGVSDRFSSSGSGTDFTLRISRVEAEDVGVYFCGQDLELPYSFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 113 - protein, 239 aa, synthetic construct
MRFSAQLLGLLVLWIPGSTADIVMTQAAFSNPVTLGTSASISCRSSKSLLQRNGITYLYWYLQKPGQSPQLLIYRMSNLASGVPDRFSSSGSGTDFTLRISRVEAEDVGVYYCGQDLELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 114 - protein, 239 aa, synthetic construct; identical to SEQ ID NO: 103

SEQ ID NO: 115 - DNA, 57 bp, synthetic construct
  source 1..57: mol_type = other DNA
atgaacttcgggctcagcttgattttccttgcccttattttaaaaggtgtccagtgt

SEQ ID NO: 116 - DNA, 54 bp, synthetic construct
  source 1..54: mol_type = other DNA
atgagagtgctgattcttttgtggctgttcacagcctttcctggtatcctgtct

SEQ ID NO: 117 - DNA, 345 bp, synthetic construct
  source 1..345: mol_type = other DNA
gaggtgcagctggtggagtctgggggagacttagtgcagcctggagggtccctgatactctcctgtgtagcctctggattcactttcagaaactatgtcatgtcttggattcgccagactccagacaagaggctggagtgggtcgcaaccattaatagtggtggtagttacacctattatcctgacactgtgaaggggcggttcaccatctccagagacaatgccaagagcaccctgtacctgcaaatgagcagtctgaagtctgaggacacagccatgtattactgtgcatctgggaattttttggactactggggtcaaggaacctcagtcaccgtctcctca

SEQ ID NO: 118 - DNA, 345 bp, synthetic construct
  source 1..345: mol_type = other DNA
gaggtgcaactggtggagtctgggggagacttagtgaagcctggagggtccctgaaactctcctgtgcagcctctggattcactttcagtaggtttgtcatgtcttgggttcgccagactccagacaagaggctggagtgggtcgcaaccattagtagtggtggtattcacacctactatctagacagtgtgaaggggcgagtcaccatcttcagagacaatgccaagaataccctgtacctgcaaatgagcagtctggagtctgaggacacagccatgtattactgtgcggctgggaatgctttggactactggggtcaaggaacctctgtcaccgtctcctca

SEQ ID NO: 119 - DNA, 345 bp, synthetic construct
  source 1..345: mol_type = other DNA
gaggtgcagctggtggagtctgggggagacttagtgcagcctggagggtccctgatactctcctgtgtagcctctggattcactttcagaaactatgtcatgtcttggattcgccagactccagacaagaggctggagtgggtcgcaaccattaatagtggtggtagttacacctattatcctgacactgtgaaggggcgattcaccatctccagagacaatgccaagagcaccctgtacctgcaaatgagcagtctgaagtctgaggacacagccatgtattactgtgcatctgggaattttttggactactggggtcaaggaacctcagtcaccgtctcctca

SEQ ID NO: 120 - DNA, 348 bp, synthetic construct
  source 1..348: mol_type = other DNA
gaggtgcacctggtggagtctgggggagacttagtgaagcctggagggtccctgaaactctcctgtgcagcctctggattcactttcagtagctatgtcatgtcttgggttcgccagactccagacaagaggctggagtggatcgcaaccattagttatggtggtggtcattacacctactatccagacagtgtgaaggggcgattcaccatctccagagacaatgccaagaacaccctgtacctgcaaatgaacagtctgaagtctgacgacacagccatgtattactgttcatctgggaatgctttgaactactggggtcaaggaacctcagtcaccgtctcctca

SEQ ID NO: 121 - DNA, 345 bp, synthetic construct
  source 1..345: mol_type = other DNA
gaggtgcaactggtggagtctgggggagacttagtgaagcctggagggtccctgaaactctcctgtgcagcctctggattcactttcagtaggaatgtcatgtcttgggttcgccagactccagacaagaggctggagtgggtcgcaaccattagcagtggtggtgttcacacctactatctagacagtgtgaaggggcgattcaccatcttcagagacaatgccaagaacaccctatacctgcaaatgaacagtctgcagtctgaggacacagccatgtattactgtgcggctgggaatgctttggactactggggtcaaggaacctctgtcaccgtctcctca

SEQ ID NO: 122 - DNA, 345 bp, synthetic construct
  source 1..345: mol_type = other DNA
gaggtgcaactggtggagtctgggggagacttagtgaagcctggagggtccctgaaactctcctgtgcagcctctggattcactttcactaactatgtcatgtcttgggttcgccagactccagacaagaggctggagtgggtcgcaaccattaatagtggtggtagatacgcctactatccagacagtgtgaaggggcgattcaccatctccagagacaatgccaagaacaccctgtacctgcaaatgagcagtctgaagtctgaggacacagccatgtattactgtgcaggggggaataatcttgactactggggccaaggcaccactctcacagtctcctca

SEQ ID NO: 123 - DNA, 345 bp, synthetic construct
  source 1..345: mol_type = other DNA
gaggtgcaactggtggagtctgggggagacttagtgaggcctggagggtccctgaaactctcctgtgcagcctctggattcattttcactaattatgtcatgtcttgggttcgccagactccagacaagaggctggagtgggtcgcaaccattaatagtgttggtagatacacctactatacagacagtgtgaaggggcgactcaccatctccagaaacaatgccaagaacaccctgtacttgcaaatgaacagtctgaagtctgaggacacagccatttattactgtgcaggggggaataatcttgacttctggggccaaggcaccactctcacagtctcctca

SEQ ID NO: 124 - DNA, 363 bp, synthetic construct
  source 1..363: mol_type = other DNA
gctgtgcagcttcaggagtcaggacctgccctggtgaaaccttctcagtcaatttcaatcacctgtactgtcactggctattccatcaccagtggttatggctggcactggatccggcagtttccaggaaacaaattggagtggatgggctacataaattacagtggtcacattgagctcaacccctctctcaaagatcgaatctctctcagtcgagacacatccaagaaccagttcttcctgcagttgatctctgtgactactgaggacacagccacatattactgtgtaagaggaggcggtctctatagtagctacggaggggactactggggtcaaggaacctcactcaccgtctcctca

SEQ ID NO: 125 - DNA, 345 bp, synthetic construct
  source 1..345: mol_type = other DNA
gaggtgcaactggtggagtctgggggagacttagtgaggcctggagggtccctgaaactctcctgtgcttcctctggattcattttcagtaactatgtcatgtcttgggttcgccagactccagacaagagactggagtgggtcgcaaccattaatagtggtggtagattcacctactatccagacagtgtgaaggggcgattcaccatctccagagacaatgccaagaacaccctgttcctgcaaatgaacagtctgaagcctgaggacacagccatatattactgtgcaggggggaataatcttgactactggggccaaggcaccactctcacagtctcctca

SEQ ID NO: 126 - DNA, 345 bp, synthetic construct
  source 1..345: mol_type = other DNA
gaggtgcagttggtggagtctgggggagacttagtgaagcctggagaatccctgaaactcgcctgtgcagcctctggattcactttcagtagggatgtcatgtcttgggttcgccagactcctgacaagaggctggagtgggtcgcaaccattagtagcaatggtatttacatctactatacagacagtgtgaagggacgattcaccatctccagagacaatgccaagaacaccctgtacctgcaactgagcagtctgaggtccgaagacacagccgtgtattactgttcaagggggaataaccttgactcctggggccaaggcaccactctcacagtctcttca

SEQ ID NO: 127 - DNA, 345 bp, synthetic construct
  source 1..345: mol_type = other DNA
gaggtgcaactggtggagtctgggggagacctggtgaagcctggagggtccctgaaactctcctgtgcagcctctggattcactttcagtaactatgtcatgtcttgggttcgccagactccagacatgaggctggagtgggtcgcaaccattaatagtggtggtagatacacctactatccagacagtgtgaaggggcgattcaccatctccagagacaatgccaagaacaccctgtacctgcaaatgaatagtctgaagtctgaggacacagccatatattactgtgcaggggggaataatcttgactactggggccaaggcaccactctcacagtctcctca

SEQ ID NO: 128 - DNA, 345 bp, synthetic construct
  source 1..345: mol_type = other DNA
gaggtgcagctggtggagtctgggggagacttagtgaagcctggagggtccctgaaactctcctgtgcagcctctggattcagtttcagtgcctatgtcatgtcttgggttcgccagactccagacaagaggctggagtgggtcgcaaccattagtcgtggtggaagttatatttactatccagacagtgtgaaggggcgattcaccatctccagagacgatgccagggacaccctgtacctgcaaatgaccagtctgaagtctgaggacacagccgtgtattactgttcaggggggaataaccttgactactggggccaaggcaccgctctcacagtctcctca

SEQ ID NO: 129 - DNA, 1008 bp, synthetic construct
  source 1..1008: mol_type = other DNA
gccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaa

SEQ ID NO: 130 - DNA, 1005 bp, synthetic construct
  source 1..1005: mol_type = other DNA
gccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 131 - DNA, 972 bp, synthetic construct
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 132 - DNA, 972 bp, synthetic construct; identical to SEQ ID NO: 131
  source 1..972: mol_type = other DNA

SEQ ID NO: 133 - DNA, 972 bp, synthetic construct; identical to SEQ ID NO: 131
  source 1..972: mol_type = other DNA

SEQ ID NO: 134 - DNA, 1008 bp, synthetic construct
  source 1..1008: mol_type = other DNA
gccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggctaagtcagaccgtcacctgcagcgttgctcacccagccagcagcaccacggtggacaaaaaacttgagcccagcgggcccatttcaacaatcaaaccctgtcctccatgcaaggagtgtcacaaatgcccagctcctaacctcgagggtggaccctccgtcttcatcttccctccaaatatcaaggatgtactcatgatctccctgacacccaaggtcacatgtgtggtggtggatgtgagtgaggatgacccagacgtccagatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgtctggtcgtgggcttcaaccctggagacatcagtgtggagtggaccagcaatgggcatacagaggagaactacaaggacaccgcaccaatcctggactctgacggttcttacttcatatatagcaagctcaatatgaaaacaagcaagtgggagaaaacagattccttctcatgcaacgtgagacacgagggtctgaaaaataactacctgaagaagaccatctcccggtctccgggtaaa

SEQ ID NO: 135 - DNA, 1008 bp, synthetic construct; identical to SEQ ID NO: 134
  source 1..1008: mol_type = other DNA

SEQ ID NO: 136 - DNA, 981 bp, synthetic construct
  source 1..981: mol_type = other DNA
gccaaaacaacagccccatcggtctatccactggcccctgtgtgtggagatacaactggctcctcggtgactctaggatgcctggtcaagggttatttccctgagccagtgaccttgacctggaactctggatccctgtccagtgatgtgcacaccttcccagctctcctgcagtctggcctctacaccctcagcagctcagtgactgtaaccacctggcccagccagaccatcacctgcaatgtggcccacccggcaagcagcaccaaagtggacaagaaaattgagcccagagggtccccaacacataaaccctgtcctccatgcccagctcctaacctcttgggtggaccatccgtcttcatcttccctccaaagatcaaggatgtactcatgatctccctgagccccatggtcacgtgtgtggtggtggatgtgagcgaggatgacccagatgtccatgtcagctggttcgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagccctcccagcgcccatcgagagaaccatctcaaaacccaaagggccagtaagagctccacaggtatatgtcttgcctccaccagaagaagagatgactaagaaacaggtcactctgacctgcatgatcacagacttcatgcctgaagacatttacgtggagtggaccaacaacgggcaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 137 - DNA, 1008 bp, synthetic construct; identical to SEQ ID NO: 134
  source 1..1008: mol_type = other DNA

SEQ ID NO: 138 - DNA, 972 bp, synthetic construct
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagtggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 139 - DNA, 1008 bp, synthetic construct; identical to SEQ ID NO: 134
  source 1..1008: mol_type = other DNA

SEQ ID NO: 140 - DNA, 972 bp, synthetic construct
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 141 - DNA, 1410 bp, synthetic construct
  source 1..1410: mol_type = other DNA
atgaacttcgggctcagcttgattttccttgcccttattttaaaaggtgtccagtgtgaggtgcagctggtggagtctgggggagacttagtgcagcctggagggtccctgatactctcctgtgtagcctctggattcactttcagaaactatgtcatgtcttggattcgccagactccagacaagaggctggagtgggtcgcaaccattaatagtggtggtagttacacctattatcctgacactgtgaaggggcggttcaccatctccagagacaatgccaagagcaccctgtacctgcaaatgagcagtctgaagtctgaggacacagccatgtattactgtgcatctgggaattttttggactactggggtcaaggaacctcagtcaccgtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaa

SEQ ID NO: 142 - DNA, 1407 bp, synthetic construct
  source 1..1407: mol_type = other DNA
atgaacttcgggctcagcttgattttccttgcccttattttaaaaggtgtccagtgtgaggtgcaactggtggagtctgggggagacttagtgaagcctggagggtccctgaaactctcctgtgcagcctctggattcactttcagtaggtttgtcatgtcttgggttcgccagactccagacaagaggctggagtgggtcgcaaccattagtagtggtggtattcacacctactatctagacagtgtgaaggggcgagtcaccatcttcagagacaatgccaagaataccctgtacctgcaaatgagcagtctggagtctgaggacacagccatgtattactgtgcggctgggaatgctttggactactggggtcaaggaacctctgtcaccgtctcctcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 143 - DNA, 1374 bp, synthetic construct
  source 1..1374: mol_type = other DNA
atgaacttcgggctcagcttgattttccttgcccttattttaaaaggtgtccagtgtgaggtgcagctggtggagtctgggggagacttagtgcagcctggagggtccctgatactctcctgtgtagcctctggattcactttcagaaactatgtcatgtcttggattcgccagactccagacaagaggctggagtgggtcgcaaccattaatagtggtggtagttacacctattatcctgacactgtgaaggggcgattcaccatctccagagacaatgccaagagcaccctgtacctgcaaatgagcagtctgaagtctgaggacacagccatgtattactgtgcatctgggaattttttggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 144 - DNA, 1377 bp, synthetic construct
  source 1..1377: mol_type = other DNA
atgaacttcgggctcagcttgatttcccttgcccttattttaaaaggtgtccagtgtgaggtgcacctggtggagtctgggggagacttagtgaagcctggagggtccctgaaactctcctgtgcagcctctggattcactttcagtagctatgtcatgtcttgggttcgccagactccagacaagaggctggagtggatcgcaaccattagttatggtggtggtcattacacctactatccagacagtgtgaaggggcgattcaccatctccagagacaatgccaagaacaccctgtacctgcaaatgaacagtctgaagtctgacgacacagccatgtattactgttcatctgggaatgctttgaactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 145 - DNA, 1410 bp, synthetic construct
  source 1..1410: mol_type = other DNA
atgaacttcgggctcagcttgattttccttgcccttattttaaaaggtctccagtgtgaggtgcaactggtggagtctgggggagacttagtgaagcctggagggtccctgaaactctcctgtgcagcctctggattcactttcagtaggaatgtcatgtcttgggttcgccagactccagacaagaggctggagtgggtcgcaaccattagcagtggtggtgttcacacctactatctagacagtgtgaaggggcgattcaccatcttcagagacaatgccaagaacaccctatacctgcaaatgaacagtctgcagtctgaggacacagccatgtattactgtgcggctgggaatgctttggactactggggtcaaggaacctctgtcaccgtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaa

SEQ ID NO: 146 - DNA, 1410 bp, synthetic construct
  source 1..1410: mol_type = other DNA
atgaacttcgggctcagcttgattttccttgcccttattttaaaaggtgtccagtgtgaggtgcaactggtggagtctgggggagacttagtgaagcctggagggtccctgaaactctcctgtgcagcctctggattcactttcactaactatgtcatgtcttgggttcgccagactccagacaagaggctggagtgggtcgcaaccattaatagtggtggtagatacgcctactatccagacagtgtgaaggggcgattcaccatctccagagacaatgccaagaacaccctgtacctgcaaatgagcagtctgaagtctgaggacacagccatgtattactgtgcaggggggaataatcttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggctaagtcagaccgtcacctgcagcgttgctcacccagccagcagcaccacggtggacaaaaaacttgagcccagcgggcccatttcaacaatcaaaccctgtcctccatgcaaggagtgtcacaaatgcccagctcctaacctcgagggtggaccctccgtcttcatcttccctccaaatatcaaggatgtactcatgatctccctgacacccaaggtcacatgtgtggtggtggatgtgagtgaggatgacccagacgtccagatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgtctggtcgtgggcttcaaccctggagacatcagtgtggagtggaccagcaatgggcatacagaggagaactacaaggacaccgcaccaatcctggactctgacggttcttacttcatatatagcaagctcaatatgaaaacaagcaagtgggagaaaacagattccttctcatgcaacgtgagacacgagggtctgaaaaataactacctgaagaagaccatctcccggtctccgggtaaa

SEQ ID NO: 147 - DNA, 1410 bp, synthetic construct
  source 1..1410: mol_type = other DNA
atgaacttcgggctcagcttgattttccttgcccttattttaaaaggtgtccagtgtgaggtgcaactggtggagtctgggggagacttagtgaggcctggagggtccctgaaactctcctgtgcagcctctggattcattttcactaattatgtcatgtcttgggttcgccagactccagacaagaggctggagtgggtcgcaaccattaatagtgttggtagatacacctactatacagacagtgtgaaggggcgactcaccatctccagaaacaatgccaagaacaccctgtacttgcaaatgaacagtctgaagtctgaggacacagccatttattactgtgcaggggggaataatcttgacttctggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggctaagtcagaccgtcacctgcagcgttgctcacccagccagcagcaccacggtggacaaaaaacttgagcccagcgggcccatttcaacaatcaaaccctgtcctccatgcaaggagtgtcacaaatgcccagctcctaacctcgagggtggaccctccgtcttcatcttccctccaaatatcaaggatgtactcatgatctccctgacacccaaggtcacatgtgtggtggtggatgtgagtgaggatgacccagacgtccagatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgtctggtcgtgggcttcaaccctggagacatcagtgtggagtggaccagcaatgggcatacagaggagaactacaaggacaccgcaccaatcctggactctgacggttcttacttcatatatagcaagctcaatatgaaaacaagcaagtgggagaaaacagattccttctcatgcaacgtgagacacgagggtctgaaaaataactacctgaagaagaccatctcccggtctccgggtaaa

SEQ ID NO: 148 - DNA, 1398 bp, synthetic construct
  source 1..1398: mol_type = other DNA
atgagagtgctgattcttttgtggctgttcacagcctttcctggtatcctgtctgctgtgcagcttcaggagtcaggacctgccctggtgaaaccttctcagtcaatttcaatcacctgtactgtcactggctattccatcaccagtggttatggctggcactggatccggcagtttccaggaaacaaattggagtggatgggctacataaattacagtggtcacattgagctcaacccctctctcaaagatcgaatctctctcagtcgagacacatccaagaaccagttcttcctgcagttgatctctgtgactactgaggacacagccacatattactgtgtaagaggaggcggtctctatagtagctacggaggggactactggggtcaaggaacctcactcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtgatgtgcacaccttcccagctctcctgcagtctggcctctacaccctcagcagctcagtgactgtaaccacctggcccagccagaccatcacctgcaatgtggcccacccggcaagcagcaccaaagtggacaagaaaattgagcccagagggtccccaacacataaaccctgtcctccatgcccagctcctaacctcttgggtggaccatccgtcttcatcttccctccaaagatcaaggatgtactcatgatctccctgagccccatggtcacgtgtgtggtggtggatgtgagcgaggatgacccagatgtccatgtcagctggttcgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagccctcccagcgcccatcgagagaaccatctcaaaacccaaagggccagtaagagctccacaggtatatgtcttgcctccaccagaagaagagatgactaagaaacaggtcactctgacctgcatgatcacagacttcatgcctgaagacatttacgtggagtggaccaacaacgggcaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 149 - DNA, 1410 bp, synthetic construct
  source 1..1410: mol_type = other DNA
atgaacttcgggctcagcttgattttccttgcccttattttaaaaggtgtccagtgtgaggtgcaactggtggagtctgggggagacttagtgaggcctggagggtccctgaaactctcctgtgcttcctctggattcattttcagtaactatgtcatgtcttgggttcgccagactccagacaagagactggagtgggtcgcaaccattaatagtggtggtagattcacctactatccagacagtgtgaaggggcgattcaccatctccagagacaatgccaagaacaccctgttcctgcaaatgaacagtctgaagcctgaggacacagccatatattactgtgcaggggggaataatcttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggctaagtcagaccgtcacctgcagcgttgctcacccagccagcagcaccacggtggacaaaaaacttgagcccagcgggcccatttcaacaatcaaaccctgtcctccatgcaaggagtgtcacaaatgcccagctcctaacctcgagggtggaccctccgtcttcatcttccctccaaatatcaaggatgtactcatgatctccctgacacccaaggtcacatgtgtggtggtggatgtgagtgaggatgacccagacgtccagatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgtctggtcgtgggcttcaaccctggagacatcagtgtggagtggaccagcaatgggcatacagaggagaactacaaggacaccgcaccaatcctggactctgacggttcttacttcatatatagcaagctcaatatgaaaacaagcaagtgggagaaaacagattccttctcatgcaacgtgagacacgagggtctgaaaaataactacctgaagaagaccatctcccggtctccgggtaaa

SEQ ID NO: 150 - DNA, 1374 bp, synthetic construct
  source 1..1374: mol_type = other DNA
atgaacttcgggctcagcttgattttccttgtccttattttaaaaggtgtccagtgtgaggtgcagttggtggagtctgggggagacttagtgaagcctggagaatccctgaaactcgcctgtgcagcctctggattcactttcagtagggatgtcatgtcttgggttcgccagactcctgacaagaggctggagtgggtcgcaaccattagtagcaatggtatttacatctactatacagacagtgtgaagggacgattcaccatctccagagacaatgccaagaacaccctgtacctgcaactgagcagtctgaggtccgaagacacagccgtgtattactgttcaagggggaataaccttgactcctggggccaaggcaccactctcacagtctcttcagccaaaacgacacccccatctgtctatccactggcccctggatctgctgcccaaactaactccatggtgaccctgggatgcctggtcaagggctatttccctgagccagtgacagtgacctggaactctggatccctgtccagtggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 151 - DNA, 1410 bp, synthetic construct
  source 1..1410: mol_type = other DNA
atgaacttcgggctcagcttgattttccttgcccttattttaaaaggtgtccagtgtgaggtgcaactggtggagtctgggggagacctggtgaagcctggagggtccctgaaactctcctgtgcagcctctggattcactttcagtaactatgtcatgtcttgggttcgccagactccagacatgaggctggagtgggtcgcaaccattaatagtggtggtagatacacctactatccagacagtgtgaaggggcgattcaccatctccagagacaatgccaagaacaccctgtacctgcaaatgaatagtctgaagtctgaggacacagccatatattactgtgcaggggggaataatcttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggctaagtcagaccgtcacctgcagcgttgctcacccagccagcagcaccacggtggacaaaaaacttgagcccagcgggcccatttcaacaatcaaaccctgtcctccatgcaaggagtgtcacaaatgcccagctcctaacctcgagggtggaccctccgtcttcatcttccctccaaatatcaaggatgtactcatgatctccctgacacccaaggtcacatgtgtggtggtggatgtgagtgaggatgacccagacgtccagatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgtctggtcgtgggcttcaaccctggagacatcagtgtggagtggaccagcaatgggcatacagaggagaactacaaggacaccgcaccaatcctggactctgacggttcttacttcatatatagcaagctcaatatgaaaacaagcaagtgggagaaaacagattccttctcatgcaacgtgagacacgagggtctgaaaaataactacctgaagaagaccatctcccggtctccgggtaaa

SEQ ID NO: 152 - DNA, 1374 bp, synthetic construct
  source 1..1374: mol_type = other DNA
atgaacttcgggctcagcttgattttccttgcccttattttaaaaggtgtccagtgtgaggtgcagctggtggagtctgggggagacttagtgaagcctggagggtccctgaaactctcctgtgcagcctctggattcagtttcagtgcctatgtcatgtcttgggttcgccagactccagacaagaggctggagtgggtcgcaaccattagtcgtggtggaagttatatttactatccagacagtgtgaaggggcgattcaccatctccagagacgatgccagggacaccctgtacctgcaaatgaccagtctgaagtctgaggacacagccgtgtattactgttcaggggggaataaccttgactactggggccaaggcaccgctctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 153 - DNA, 60 bp, synthetic construct
  source 1..60: mol_type = other DNA
atgaggttctctgctcagcttctggggctgcttgtgctctggatccctggatccactgca

SEQ ID NO: 154 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctaaatagttatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaagatctagaacttccgtatacgttcggatcggggacccagctggaaataaaa

SEQ ID NO: 155 - DNA, 336 bp, synthetic construct
  source 1..336: mol_type = other DNA
gatattgtgatgacgcaggctgcattctccaatccagtcactcttggaacatcaggttccgtctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcactgggtcaggaactgatttcacactgagaatcagcagagtggaggctgaggatgtgggtgtttattactgtggtcaagatctagaacttccgtattcgttcggatcggggaccaagctggaaataaaa

SEQ ID NO: 156 - DNA, 336 bp, synthetic construct; identical to SEQ ID NO: 154
  source 1..336: mol_type = other DNA

SEQ ID NO: 157 - DNA, 336 bp, synthetic construct
  source 1..336: mol_type = other DNA
gatattgtgatgacgcaggctgcattctccaatccagtcactcttggaacatcagcttccatctcctgtaggtctagtaagagtctcctacattatactggcatcacttatttgtactggtatttgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaagatctagaacttccgtattcgttcggatcggggaccaagctggaaataaaa

SEQ ID NO: 158 - DNA, 336 bp, synthetic construct
  source 1..336: mol_type = other DNA
gatattatgatgacgcaggctgcattctccaatccagtcactcttggaacatcaggttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaggatctagaacttccgtattcgttcggatcggggaccaagctggaaataaaa

SEQ ID NO: 159 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctacagagaaatggcatcacttatttgtattggtatctgcagaagccaggccagtctcctcagctcctgatttatcgactgtccaaccttgcctcaggagtcccagacaggttcagtagcagtgggtcaggaactgatttcacactgagaatcagcagagtggaggctgaggatgtgggtgtttattactgtggtcaagatctggaacttccgtacacgttcggaggggggaccaagctggaaataaaa

SEQ ID NO: 160 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaagatctagaacttccgtactcgttcggaggggggaccaagctggaaataaaa

SEQ ID NO: 161 - DNA, 321 bp, synthetic construct
  source 1..321: mol_type = other DNA
gacattcagatgacccagtctcctgcctcccagtctgcatctctgggagaaagtgtcaccatcacatgcctggcaagtcaaaccattggtacatggttagcatggtatcatcagaaacctgggacatctcctcagctcctgatttatggtgcaatcaggttggcagatggggtcccgtcaagattcagtggtggtggatctggcacagagttttcgctcaagatcagcagcctacaggccgaagattttggcagttattactgtcaacaactttacaggagtccgtacgcgttcggaggggggaccaacctggaaataaaa

SEQ ID NO: 162 - DNA, 336 bp, synthetic construct
  source 1..336: mol_type = other DNA
gatattgtgatgacgcaggctgaattctccaatccagtcactcttggaacatcagcttccatctcctgcaggtctagtaagagtctcctacagagaagtggcatcacttacttgtattggtatcttcagaagccaggccagtctcctcagctcctgatttatcgaatgtccaaccttgcctcaggagtcccagacaggttcagtagcagtgggtcaggcactgatttcacactgagaatcagcagagtggaggctgaggatgtgggtgtttattactgtggtcaagatatggaacttccgtacacgttcggaggggggaccaagctggaaataaaa

SEQ ID NO: 163 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctacatagaaatggcatcacttatttgtattggtatctgcagaagccaggccagtctcctcagctcctgatttatcagatgtccaaccttgcctcaggagtctcagacaggttcagtagcagtgggtcaggaactgatttcacactgagaatcagcagagtggaggctgaggatgtgggtgtttatttctgtggtcaagatctagaacttccatactcgttcggaggggggaccaagctggaaataaaa

SEQ ID NO: 164 - DNA, 336 bp, synthetic construct
  source 1..336: mol_type = other DNA
gatattgtgatgacgcaggctgcattctccaatccagtcactcttggaacatcagcttccatctcctgcaggtctagtaagagtctcctacagagaaatggcatcacttatttgtattggtatctgcagaagccaggccagtctcctcagctcctgatttatcggatgtccaaccttgcctcaggagtcccagacaggttcagtagcagtgggtcaggaactgatttcacactgagaatcagcagagtggaggctgaggatgtgggtgtttattactgtggtcaagatctggaacttccgtacacgttcggaggggggaccaagctggaaataaaa

SEQ ID NO: 165 - DNA, 336 bp, synthetic construct
  source 1..336: mol_type = other DNA
gatattgtgatgacgcaggctgccttctccaatccagtcactcttggaacatcagcttccatttcctgcaggtctagtaagagtctcctacaaattaatggcatcacttatttgtattggtatctgcagaagccaggccagtctcctcagctcctgatttatcagatgtccaaccttgcctcaggagtcccagataggttcagtagcagtgggtcaggaactgatttcacactgagaatcagcagagtggaggctgaggatgtgggtgtttattactgtggccaagatctagaacttccgtacacgttcggaggggggaccaagctggaaataaaa

SEQ ID NO: 166 - DNA, 321 bp, synthetic construct
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt

SEQ ID NO: 167 - DNA, 717 bp, synthetic construct
  source 1..717: mol_type = other DNA
atgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctaaatagttatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaagatctagaacttccgtatacgttcggatcggggacccagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt

SEQ ID NO: 168 - DNA, 717 bp, synthetic construct
  source 1..717: mol_type = other DNA
atgaggttctctgctcagcttctggggctgcttgtgctctggatccctggatccactgcagatattgtgatgacgcaggctgcattctccaatccagtcactcttggaacatcaggttccgtctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcactgggtcaggaactgatttcacactgagaatcagcagagtggaggctgaggatgtgggtgtttattactgtggtcaagatctagaacttccgtattcgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt

SEQ ID NO: 169 - DNA, 717 bp, synthetic construct; identical to SEQ ID NO: 167
  source 1..717: mol_type = other DNA

SEQ ID NO: 170 - DNA, 717 bp, synthetic construct
  source 1..717: mol_type = other DNA
atgaggttctctgctcagcttctggggctgcttgtgctctggatccctggatccactgcagatattgtgatgacgcaggctgcattctccaatccagtcactcttggaacatcagcttccatctcctgtaggtctagtaagagtctcctacattatactggcatcacttatttgtactggtatttgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaagatctagaacttccgtattcgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt

SEQ ID NO: 171 - DNA, 717 bp, synthetic construct
  source 1..717: mol_type = other DNA
atgaggttctctgctcagcttctggggctgcttgtgctctggatccctggatccactgcagatattatgatgacgcaggctgcattctccaatccagtcactcttggaacatcaggttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaggatctagaacttccgtattcgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt

SEQ ID NO: 172 - DNA, 717 bp, synthetic construct
  source 1..717: mol_type = other DNA
atgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctacagagaaatggcatcacttatttgtattggtatctgcagaagccaggccagtctcctcagctcctgatttatcgactgtccaaccttgcctcaggagtcccagacaggttcagtagcagtgggtcaggaactgatttcacactgagaatcagcagagtggaggctgaggatgtgggtgtttattactgtggtcaagatctggaacttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgt

SEQ ID NO: 173 - DNA, 717 bp, synthetic construct
  misc_feature 1..717: note = Leader Chain full sequence (178K 1F4)
  source 1..717: mol_type = other DNA
atgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtggtcaagatctagaacttccgtactcgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgt

SEQ ID NO: 174 - DNA, 702 bp, synthetic construct
  source 1..702: mol_type = other DNA
atgaggttctctgctcagcttctggggctgcttgtgctctggatccctggatccactgcagacattcagatgacccagtctcctgcctcccagtctgcatctctgggagaaagtgtcaccatcacatgcctggcaagtcaaaccattggtacatggttagcatggtatcatcagaaacctgggacatctcctcagctcctgatttatggtgcaatcaggttggcagatggggtcccgtcaagattcagtggtggtggatctggcacagagttttcgctcaagatcagcagcctacaggccgaagattttggcagttattactgtcaacaactttacaggagtccgtacgcgttcggaggggggaccaacctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgt

SEQ ID NO: 175 - DNA, 720 bp, synthetic construct
  source 1..720: mol_type = other DNA
atgaggttctctgctcagcttctggggctgcttgtgctctggatccctggatccactgcagatattgtgatgacgcaggctgaattctccaatccagtcactcttggaacatcagcttccatctcctgcaggtctagtaagagtctcctacagagaagtggcatcacttacttgtattggtatcttcagaagccaggccagtctcctcagctcctgatttatcgaatgtccaaccttgcctcaggagtcccagacaggttcagtagcagtgggtcaggcactgatttcacactgagaatcagcagagtggaggctgaggatgtgggtgtttattactgtggtcaagatatggaacttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgttgt

SEQ ID NO: 176 - DNA, 717 bp, synthetic construct
  source 1..717: mol_type = other DNA
atgaggttctctgctcagcttctggggctgcttgtgctctggatccctggatcaactgcagatattgtgatgacgcaggctgcattctccaatccagtcactcttggaacatcagcttccatctcctgcaggtctagtaagagtctcctacatagaaatggcatcacttatttgtattggtatctgcagaagccaggccagtctcctcagctcctgatttatcagatgtccaaccttgcctcaggagtctcagacaggttcagtagcagtgggtcaggaactgatttcacactgagaatcagcagagtggaggctgaggatgtgggtgtttatttctgtggtcaagatctagaacttccatactcgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgt

SEQ ID NO: 177 - DNA, 717 bp, synthetic construct
  source 1..717: mol_type = other DNA
atgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctacagagaaatggcatcacttatttgtattggtatctgcagaagccaggccagtctcctcagctcctgatttatcggatgtccaaccttgcctcaggagtcccagacaggttcagtagcagtgggtcaggaactgatttcacactgagaatcagcagagtggaggctgaggatgtgggtgtttattactgtggtcaagatctggaacttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgt

SEQ ID NO: 178 - DNA, 717 bp, synthetic construct
  source 1..717: mol_type = other DNA
atgaggttctctgctcagcttctggggctgcttgtgctctggatccctggatccactgcagatattgtgatgacgcaggctgccttctccaatccagtcactcttggaacatcagcttccatttcctgcaggtctagtaagagtctcctacaaattaatggcatcacttatttgtattggtatctgcagaagccaggccagtctcctcagctcctgatttatcagatgtccaaccttgcctcaggagtcccagataggttcagtagcagtgggtcaggaactgatttcacactgagaatcagcagagtggaggctgaggatgtgggtgtttattactgtggccaagatctagaacttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgt

SEQ ID NO: 179 - protein, 6 aa, synthetic construct
GNYLDY

SEQ ID NO: 180 - protein, 16 aa, synthetic construct
RSSKSLLQRNGITYLH

SEQ ID NO: 181 - protein, 336 aa, synthetic construct; identical to SEQ ID NO: 59

SEQ ID NO: 182 - protein, 115 aa, synthetic construct
EVQLVESGGDFVKPGGSLKLSCAASGFTFSNYVMSWVRQTPDKRLEWVATINSGGRYTYYPDSVKGRFTISRDNAKNTLYLQMNSLKSDDTAIYYCSGGNYLDYWGQGTTLTVSS

SEQ ID NO: 183 - protein, 470 aa, synthetic construct
MNFGLSLIFLALILKGVQCEVQLVESGGDFVKPGGSLKLSCAASGFTFSNYVMSWVRQTPDKRLEWVATINSGGRYTYYPDSVKGRFTISRDNAKNTLYLQMNSLKSDDTAIYYCSGGNYLDYWGQGTTLTVSSAKTTPPSVYPLAPGCGDTTGSSVTLGCLVKGYFPESVTVTWNSGSLSSSVHTFPALLQSGLYTMSSSVTVPSSTWLSQTVTCSVAHPASSTTVDKKLEPSGPISTIKPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPILDSDGSYFIYSKLNMKTSKWEKTDSFSCNVRHEGLKNNYLKKTISRSPGK

SEQ ID NO: 184 - protein, 112 aa, synthetic construct
DIVMTQAAFSNPVTLGTSASISCRSSKSLLQRNGITYLHWYLQKPGQSPQLLIYRMSNLASGVPERFSSSGSGTDFTLRISRVEAEDVGVYYCGQDLELPYTFGGGTKLEIK

SEQ ID NO: 185 - protein, 239 aa, synthetic construct
MRFSAQLLGLLVLWIPGSTADIVMTQAAFSNPVTLGTSASISCRSSKSLLQRNGITYLHWYLQKPGQSPQLLIYRMSNLASGVPERFSSSGSGTDFTLRISRVEAEDVGVYYCGQDLELPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 186 - DNA, 345 bp, synthetic construct
  source 1..345: mol_type = other DNA
gaggtgcaactggtggagtctgggggagacttcgtgaagcctggagggtccctgaaactctcctgtgcagcctctggattcactttcagtaattatgtcatgtcttgggttcgccagactccagacaagaggctggagtgggtcgcaaccattaatagtggtggtagatacacctactatccagacagtgtgaaggggcgattcaccatttccagagacaatgccaagaacaccctgtacctgcaaatgaacagtctgaagtctgatgacacagccatatattactgttcaggggggaattatcttgactactggggccaaggcaccactctcacagtctcctca

SEQ ID NO: 187 - DNA, 1008 bp, synthetic construct; identical to SEQ ID NO: 134
  source 1..1008: mol_type = other DNA

SEQ ID NO: 188 - DNA, 1410 bp, synthetic construct
  source 1..1410: mol_type = other DNA
atgaacttcgggctcagcttgattttccttgcccttattttaaaaggtgtccagtgtgaggtgcaactggtggagtctgggggagacttcgtgaagcctggagggtccctgaaactctcctgtgcagcctctggattcactttcagtaattatgtcatgtcttgggttcgccagactccagacaagaggctggagtgggtcgcaaccattaatagtggtggtagatacacctactatccagacagtgtgaaggggcgattcaccatttccagagacaatgccaagaacaccctgtacctgcaaatgaacagtctgaagtctgatgacacagccatatattactgttcaggggggaattatcttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggctaagtcagaccgtcacctgcagcgttgctcacccagccagcagcaccacggtggacaaaaaacttgagcccagcgggcccatttcaacaatcaaaccctgtcctccatgcaaggagtgtcacaaatgcccagctcctaacctcgagggtggaccctccgtcttcatcttccctccaaatatcaaggatgtactcatgatctccctgacacccaaggtcacatgtgtggtggtggatgtgagtgaggatgacccagacgtccagatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgtctggtcgtgggcttcaaccctggagacatcagtgtggagtggaccagcaatgggcatacagaggagaactacaaggacaccgcaccaatcctggactctgacggttcttacttcatatatagcaagctcaatatgaaaacaagcaagtgggagaaaacagattccttctcatgcaacgtgagacacgagggtctgaaaaataactacctgaagaagaccatctcccggtctccgggtaaa

SEQ ID NO: 189 - DNA, 396 bp, synthetic construct
  source 1..396: mol_type = other DNA
atgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctccagagaaatggcatcacttatttgcattggtatctgcagaagccaggccagtctcctcagctcctgatttatcgaatgtccaaccttgcctcaggagtcccagaaaggttcagtagcagtgggtcaggaactgatttcacactgagaatcagcagagtggaggctgaggatgtgggtgtttattactgtggtcaagatctggaacttccgtacacgttcggaggggggaccaagctggaaataaaa

SEQ ID NO: 190 - DNA, 717 bp, synthetic construct
  source 1..717: mol_type = other DNA
atgaggttctctgctcagcttctggggctgcttgtgctctggatccctggatccactgcagatattgtgatgacgcaggctgcattctccaatccagtcactcttggaacatcagcttccatctcctgcaggtctagtaagagtctcctccagagaaatggcatcacttatttgcattggtatctgcagaagccaggccagtctcctcagctcctgatttatcgaatgtccaaccttgcctcaggagtcccagaaaggttcagtagcagtgggtcaggaactgatttcacactgagaatcagcagagtggaggctgaggatgtgggtgtttattactgtggtcaagatctggaacttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacattgaccaaggacgagtatgaacgacataacagctatacctgtgaggccactcacaagacatcaacttcacccattgtcaagagcttcaacaggaatgagtgt

SEQ ID NO: 191 - DNA, 15 bp, synthetic construct
  source 1..15: mol_type = other DNA
aactatgtcatgtct

SEQ ID NO: 192 - DNA, 15 bp, synthetic construct
  source 1..15: mol_type = other DNA
aggtttgtcatgtct

SEQ ID NO: 193 - DNA, 15 bp, synthetic construct
  source 1..15: mol_type = other DNA
agctatgtcatgtct

SEQ ID NO: 194 - DNA, 15 bp, synthetic construct
  source 1..15: mol_type = other DNA
aggaatgtcatgtct

SEQ ID NO: 195 - DNA, 15 bp, synthetic construct
  source 1..15: mol_type = other DNA
agggatgtcatgtct

SEQ ID NO: 196 - DNA, 15 bp, synthetic construct
  source 1..15: mol_type = other DNA
gcctatgtcatgtct

SEQ ID NO: 197 - DNA, 51 bp, synthetic construct
  source 1..51: mol_type = other DNA
accattaatagtggtggtagttacacctattatcctgacactgtgaagggg

SEQ ID NO: 198 - DNA, 51 bp, synthetic construct
  source 1..51: mol_type = other DNA
accattagtagtggtggtattcacacctactatctagacagtgtgaagggg

SEQ ID NO: 199 - DNA, 54 bp, synthetic construct
  source 1..54: mol_type = other DNA
accattagttatggtggtggtcattacacctactatccagacagtgtgaagggg

SEQ ID NO: 200 - DNA, 51 bp, synthetic construct
  source 1..51: mol_type = other DNA
accattagcagtggtggtgttcacacctactatctagacagtgtgaagggg

SEQ ID NO: 201 - DNA, 51 bp, synthetic construct
  source 1..51: mol_type = other DNA
accattaatagtggtggtagatacgcctactatccagacagtgtgaagggg

SEQ ID NO: 202 - DNA, 51 bp, synthetic construct
  source 1..51: mol_type = other DNA
accattaatagtgttggtagatacacctactatacagacagtgtgaagggg

SEQ ID NO: 203 - DNA, 48 bp, synthetic construct
  source 1..48: mol_type = other DNA
tacataaattacagtggtcacattgagctcaacccctctctcaaagat

SEQ ID NO: 204 - DNA, 51 bp, synthetic construct
  source 1..51: mol_type = other DNA
accattaatagtggtggtagattcacctactatccagacagtgtgaagggg

SEQ ID NO: 205 - DNA, 51 bp, synthetic construct
  source 1..51: mol_type = other DNA
accattagtagcaatggtatttacatctactatacagacagtgtgaaggga

SEQ ID NO: 206 - DNA, 51 bp, synthetic construct
  source 1..51: mol_type = other DNA
accattaatagtggtggtagatacacctactatccagacagtgtgaagggg

SEQ ID NO: 207 - DNA, 51 bp, synthetic construct
  source 1..51: mol_type = other DNA
accattagtcgtggtggaagttatatttactatccagacagtgtgaagggg

SEQ ID NO: 208 - DNA, 18 bp, synthetic construct
  source 1..18: mol_type = other DNA
gggaattttttggactac

SEQ ID NO: 209 - DNA, 18 bp, synthetic construct
  source 1..18: mol_type = other DNA
gggaatgctttggactac

SEQ ID NO: 210 - DNA, 18 bp, synthetic construct
  source 1..18: mol_type = other DNA
gggaattttttggactac

SEQ ID NO: 211 - DNA, 18 bp, synthetic construct
  source 1..18: mol_type = other DNA
gggaatgctttgaactac

SEQ ID NO: 212 - DNA, 18 bp, synthetic construct
  source 1..18: mol_type = other DNA
gggaataatcttgactac

SEQ ID NO: 213 - DNA, 18 bp, synthetic construct
  source 1..18: mol_type = other DNA
gggaataatcttgacttc

SEQ ID NO: 214 - DNA, 36 bp, synthetic construct
  source 1..36: mol_type = other DNA
ggaggcggtctctatagtagctacggaggggactac

SEQ ID NO: 215 - DNA, 18 bp, synthetic construct
  source 1..18: mol_type = other DNA
gggaataaccttgactcc

SEQ ID NO: 216 - DNA, 48 bp, synthetic construct
  source 1..48: mol_type = other DNA
aggtctagtaagagtctcctaaatagttatggcatcacttatttgtat

SEQ ID NO: 217 - DNA, 47 bp, synthetic construct
  source 1..47: mol_type = other DNA
aggtctagtaagagtctcctacatagtaatggcatcacttatttgta

SEQ ID NO: 218 - DNA, 48 bp, synthetic construct
  source 1..48: mol_type = other DNA
aggtctagtaagagtctcctacattatactggcatcacttatttgtac

SEQ ID NO: 219 - DNA, 48 bp, synthetic construct
  source 1..48: mol_type = other DNA
aggtctagtaagagtctcctacatagtaatggcatcacttatttgtat

SEQ ID NO: 220 - DNA, 33 bp, synthetic construct
  source 1..33: mol_type = other DNA
ctggcaagtcaaaccattggtacatggttagca

SEQ ID NO: 221 - DNA, 48 bp, synthetic construct
  source 1..48: mol_type = other DNA
aggtctagtaagagtctcctacagagaagtggcatcacttacttgtat

SEQ ID NO: 222 - DNA, 48 bp, synthetic construct
  source 1..48: mol_type = other DNA
aggtctagtaagagtctcctacatagaaatggcatcacttatttgtat

SEQ ID NO: 223 - DNA, 48 bp, synthetic construct
  source 1..48: mol_type = other DNA
aggtctagtaagagtctcctacaaattaatggcatcacttatttgtat

SEQ ID NO: 224 - DNA, 21 bp, synthetic construct
  source 1..21: mol_type = other DNA
cagatgtccaaccttgcctca

SEQ ID NO: 225 - DNA, 21 bp, synthetic construct
  source 1..21: mol_type = other DNA
cgactgtccaaccttgcctca

SEQ ID NO: 226 - DNA, 21 bp, synthetic construct
  source 1..21: mol_type = other DNA
ggtgcaatcaggttggcagat

SEQ ID NO: 227 - DNA, 21 bp, synthetic construct
  source 1..21: mol_type = other DNA
cgaatgtccaaccttgcctca

SEQ ID NO: 228 - DNA, 27 bp, synthetic construct
  source 1..27: mol_type = other DNA
ggtcaagatctagaacttccgtatacg

SEQ ID NO: 229 - DNA, 27 bp, synthetic construct
  source 1..27: mol_type = other DNA
ggtcaagatctagaacttccgtattcg

SEQ ID NO: 230 - DNA, 27 bp, synthetic construct
  source 1..27: mol_type = other DNA
caacaactttacaggagtccgtacgcg

SEQ ID NO: 231 - DNA, 27 bp, synthetic construct
  source 1..27: mol_type = other DNA
ggtcaagatatggaacttccgtacacg

(19) United States
(12) Patent Application Publication
(10) Pub. No.: US 2026/0234121 A1
(43) Pub. Date: Aug. 13, 2026

(54) DERIVATIVES, IMMUNOGENS, AND ANTIBODIES AND USE OF THE SAME IN DETECTION OF ECSTASY-CLASS COMPOUNDS

(71) Applicant: Siemens Healthcare Diagnostics Inc., Tarrytown, NY (US)

(72) Inventors: Bogdan DRAGHICI, Newark, DE (US); Tatiana MAREEVA, Wilmington, DE (US); Zhijian ZHAO, Wilmington, DE (US); Yi Feng ZHENG, Wilmington, DE (US); Yuriy SMIRNOV, Wilmington, DE (US); Manoj SHARMA, Hockessin, DE (US); Heungyeung YAU, Wilmington, DE (US); Nihmat MORJANA, Newark, DE (US); William BEDZYK, Odessa, DE (US)

(21) Appl. No.: 19/153,643

(22) PCT Filed: Feb. 6, 2024
(86) PCT No.: PCT/US2024/014552

§ 371 Date: Aug. 5, 2025

Related U.S. Application Data
(60) Provisional application No. 63/484,281, filed on Feb. 10, 2023.

(51) Int. Cl. C07D 317/58; C07K 16/44; G01N 33/535; G01N 33/94

(52) U.S. Cl. CPC C07D 317/58; C07K 16/44; G01N 33/535; G01N 33/94; C07K 2317/565; C07K 2317/92

(57) ABSTRACT

Haptens and immunogens for ecstasy-class compounds (enactogens) and antibodies raised against the haptens and immunogens are provided herein. The haptens and immunogens include MDA or derivatives thereof conjugated via a linker to an immunogenic carrier or a label. The antibodies raised against the haptens and immunogens recognize MDA and MDMA with the same or similar efficiency and do not cross-react or exhibit limited cross-reactivity to non-ecstasy-class compounds. Also provided are kits including the haptens or immunogens and antibodies raised against the compounds as well as methods for detecting ecstasy-class compounds in a sample.

This application incorporates by reference the sequence listing which is submitted together with this application in computer readable form which has the file name 2021P08844WO_SeqList.XML and is 291 KB.

### FIELD

This disclosure generally relates to methods, compositions, and kits for detecting the presence and/or amounts of entactogens in biological samples. In particular, the disclosure relates to haptens, immunogens, and assays for 3,4-methylenedioxy-methamphetamine (MDMA), 3,4-methylenedioxyamphetamine (MDA), and related compounds.

### BACKGROUND

This section provides background information related to the present disclosure which is not necessarily prior art.

The clinical diagnostic field has seen a broad expansion in recent years, both as to the variety of materials of interest that may be readily and accurately determined, as well as the methods for the determination. Over the last decade, testing for drugs of abuse has become commonplace. This testing is not only for the monitoring of criminal offenders and drug addicts, but employers also use it for the screening of workers. In recent years, immunoassays based on a reaction of an antibody with an antigen have been extensively investigated for this purpose. Immunoassays may be roughly classified into a radioimmunoassay using a radioactive isotope, an enzyme-immunoassay (EIA) using an enzyme, and a luminescence assay using fluorescent labels, e.g., fluorescence polarization, and chemiluminescent labels.

Amphetamine and methamphetamine stimulate the central nervous system and have been used medicinally to treat hypotension, narcolepsy and obesity. Because of their stimulating effects, the drugs and derivatives have been abused.

The use and abuse of a class of illicit designer drugs known commonly as “ecstasy drugs” have increased significantly in recent years. One such class of designer drugs is ecstasy-class compounds or drugs. Non-limiting examples of compounds in this class include, 3,4-methylenedioxymethamphetamine (MDMA), also known as “ecstasy”, 3,4-methylenedioxyamphetamine (MDA), N-ethyl-3,4-methylenedioxyamphetamine (MDE), methylenedioxyethylamphetamine (MDEA), N-methyl-1-(3,4-methylenedioxyphenyl)-2-butanamine (MBDB), 1-(3,4-methylenedioxyphenyl)-2-butanamine (BDB), 3,4-methylenedioxy-N-propylamphetamine (MDPA), and other derivatives of amphetamine. As drug designers develop more and more variants of ecstasy, the number of unique compounds which falls within the ecstasy-class continues to grow.

Detection of ecstasy-class compounds, such as MDMA, MDA, MDEA, MDE, MBDB, and BDB or a derivative or metabolite thereof, in urine currently depends upon cross-reactivity of such ecstasy-class drugs in immunoassays for amphetamine and methamphetamine. These assays, however, fail to detect such ecstasy-class compounds at lower concentrations. Also, new Substance Abuse and Mental Health Services Administration (SAMHSA) guidelines require an MDA and MDMA recovery of 80% or greater and the capacity to distinguish MDA and MDMA from related structurally compounds, such as amphetamine and methamphetamine. Existing immunoassays cannot reliably recover 80% or greater of MDA or MDMA in a sample. Moreover, existing immunoassays for amphetamine and methamphetamine are limited by their cross-reactivity to over-the-counter allergy and cold medications, such as (±) ephedrine, (±) pseudoephedrine, and phenylpropanolamine, and to prescription diet drugs such as phentermine. This cross-reactivity factor prevents the lowering of the cut-off level for detection of amphetamine and methamphetamine, which, in turn, prevents detecting ecstasy-class compounds at lower concentrations. Therefore, an assay with increased specificity for ecstasy-class compounds is needed, either as an assay to detect ecstasy-class compounds alone, or as an assay to detect ecstasy-class compounds as distinguished from amphetamine and methamphetamine.

### SUMMARY

This section provides a general summary of the disclosure and is not a comprehensive disclosure of its full scope or all of its features.

In certain aspects, the present disclosure provides a compound corresponding in structure to a Formula (I):

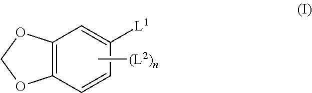

wherein
   L¹ is

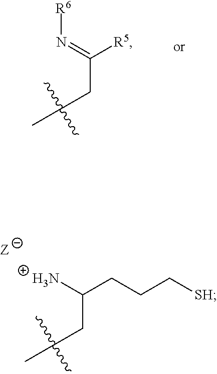

R⁵ is hydrogen or an alkyl; R⁶ is hydrogen, an alkyl, or —(X⁴)—(Y⁴)ₕ; X⁴ is —SH, —NH₂, —COOH, —CONH—, —O(CH₂)(CO)(NH)(C₂H₄)(NH)(CO)(CH₂)Br, or —O(CH₂)(CO)(NH)(C₂H₄)(NH)(CO)(CH₂)S—; Y⁴ is an immunogenic carrier or a label; h is zero or 1; a is zero or 1; L² is —(CH₂)ᵢ—(X⁵)(Y⁵)ⱼ; X⁵ is —SH, —NH₂, —COOH, or —S(CH₂)(CO)NH—, Y⁵ is an immunogenic carrier or a label; i is 1 to 12; j is zero or 1; Z is Cl, (CF₃)CO₂, F, or Br.

In certain aspects, the present disclosure provides a compound corresponding in structure to a Formula (I):

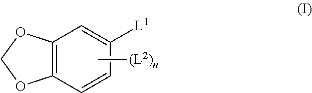

wherein
   L¹ is

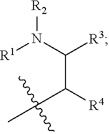

R¹ is hydrogen, an alkyl, or —CO(CF₃); R² is hydrogen, an alkyl, or —(CH₂)_{b}—(X¹)—(Y¹)_{c}; X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or

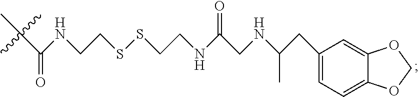 [SMILES: *C(=O)NCCSSCCNC(=O)CNC(C)Cc1ccc2c(c1)OCO2]

Y¹ is an immunogenic carrier or a label; b is 1 to 10; c is zero or 1; R³ is hydrogen, alkyl, or —(CH₂)_{d}—(X²)—(Y²)ₑ; X² is —SH, —NH₂, —COOH, —CONH—, —S(CH₂)(CO)NH—, —CO(NH)(C₂H₄)S(CH₂)(CO)NH—, or

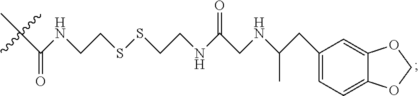 [SMILES: *C(=O)NCCSSCCNC(=O)CNC(C)Cc1ccc2c(c1)OCO2]

Y² is an immunogenic carrier or a label; d is 2 to 12; e is zero or 1; R⁴ is hydrogen or —(CH₂)_{f}(X³)—(Y³)_{g}; X³ is —SH, —NH₂, —COOH, —CONH—, —S(CH₂)(CO)NH—, —CO(NH)(C₂H₄)S(CH₂)(CO)NH—, or

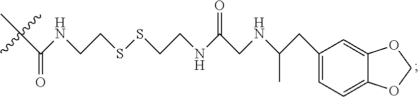 [SMILES: *C(=O)NCCSSCCNC(=O)CNC(C)Cc1ccc2c(c1)OCO2]

Y³ is an immunogenic carrier or a label; f is 1 to 12; g is zero or 1; a is zero or 1; L² is —(CH₂)ᵢ—(X⁵)—(Y⁵)ⱼ; X⁵ is —SH, —NH₂, —COOH, or —S(CH₂)(CO)NH—, Y⁵ is an immunogenic carrier or a label; i is 1 to 12; and j is zero or 1; wherein when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ is hydrogen; wherein when a is zero, R¹ is hydrogen or methyl, R² is —(CH₂)_{b}—(X¹)—(Y¹)_{c}, X¹ is —CONH—, c is 1, Y¹ is an immunogenic carrier or a label, R³ is methyl, R⁴ is hydrogen, then b is 5-10; wherein when a is zero, R² is —(CH₂)_{b}—(X¹)—(Y¹)_{c}, b is 3 or 4, X¹ is —COOH, c is zero, R³ is methyl, R⁴ is hydrogen, then R¹ is hydrogen; wherein when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b is 5-10; and wherein at least one of R¹, R², and R³ is neither hydrogen nor an alkyl.

In yet other aspects, the present disclosure provides a compound having the structure

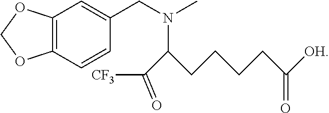 [SMILES: CN(Cc1ccc2c(c1)OCO2)C(CCCCC(=O)O)C(=O)C(F)(F)F]

In yet other aspects, the present disclosure provides an antibody raised against a compound as described herein, wherein the immunogenic carrier is present. The antibody is useful for detecting an ecstasy-class compound in an immunoassay.

In yet other aspects, the present disclosure provides a polynucleotide including a DNA sequence encoding an antibody as described herein. The DNA sequences may encode the light or heavy chains, or any portion thereof.

In yet other aspects, the present disclosure provides a kit including one or more antibodies described herein and a conjugate of an enzyme and an MDA analog and/or a conjugate of an enzyme and an MDMA analog.

In yet other aspects, the present disclosure provides a method for determining the presence of MDA and/or MDMA in a sample suspected of containing MDA and/or MDMA. The method includes providing a sample, a conjugate of an enzyme and either the MDA analog or MDMA analog, and an antibody disclosed herein to a medium and examining the medium for the presence of a complex comprising the MDA and/or MDMA and the antibody, wherein the conjugate corresponds in structure to Formula (I) as described herein.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a reaction scheme 1 depicting an example of a synthesis of a E5 hapten (9) and activated E5 hapten (10).

FIG. 2 is a reaction scheme 2 depicting an example of a synthesis of E5-OVA conjugate (44), E5-cBSA1 conjugate (45), cBSA2 conjugate (46), and E5-KLH immunogen (47).

FIG. 3 is a reaction scheme 3 depicting an example of a synthesis of an E1 hapten (51), activated E1 hapten (52), E1-OVA conjugate (55), and E1-KLH immunogen (56).

FIG. 4 is a reaction scheme 4 depicting an example of a synthesis of an E2 hapten (60), activated E2 hapten (61), OVA conjugate (65), 1000×KLH immunogen (66), and 180×KLH immunogen (67).

FIG. 5 is a reaction scheme 5 depicting an example of a synthesis of an E3 hapten (70), activated E3 hapten (71), E3-OVA conjugate (72), and E3-KLH immunogen (73).

FIG. 6 is a reaction scheme 6 depicting an example of a synthesis of an E1-G6PDH conjugates (58a), (58b), (58c), (59a), (59b), and 59 (c).

FIG. 7A is a reaction scheme 7a depicting an example of a synthesis of an E3-G6PDH conjugates (74a), (74b), (74c), (74d), and (74e).

FIG. 7B is a reaction scheme 7b depicting an example of a synthesis of an activated E2 hapten (61) and an E2-G6PDH conjugates (68a), (68b), (68c), (68d), and (68e).

FIG. 8 is a reaction scheme 8 depicting an example of a synthesis of an E4 hapten (81) and E4-G6PDH conjugate (82).

FIG. 9A is a reaction scheme 9a depicting an example of a synthesis of a E6 hapten (19) and activated E6 hapten (20).

FIG. 9B is a reaction scheme 9b depicting an example of a synthesis of an activated E5 hapten (22).

FIG. 9C is a reaction scheme 9c depicting an example of a synthesis of E5-G6PDH conjugates (24a), (24b), (24c), (26a), (26b), (26c) and E6-G6PDH conjugates (25a), (25b), (25c), (27a), (27b), and (27c).

FIG. 10 shows the reactivity of E1-KLH (56) (A and B), E2-KLH (66) (C and D), and E3-KLH (73) (E and F) specific polyclonal antibodies toward MDMA and MDA. The exemplary results are of competitive assay performed on E1-OVA (55) coated ELISA plates with rabbit (A, C, and E) and mouse (B, D, and F) antisera. Rabbit and mice polyclonal antibodies generated against E2-KLH (66) or E3-KLH (72) immunogen preferably bind MDMA over MDA. Only antisera of E1-KLH (56) immunized animals (A and B) demonstrated the same sensitivity to both compounds.

FIG. 11 shows antigen-binding properties of rabbit polyclonal (A) and mouse monoclonal (B-L) antibodies generated against E1-KLH (56). Competitive ELISA performed on E1-OVA (55) coated plates for MDMA, MDA, amphetamine (Amph) and methamphetamine (mAmph) demonstrate that these antibodies recognize MDMA and MDA with efficiency≥80% and distinguish ecstasy class drugs from structurally-related compounds such as amphetamine and methamphetamine.

FIG. 12 depicts a comparison of antigen-binding properties of exemplary anti-E1-KLH rabbit polyclonal (A, E, and I) and mouse monoclonal (B-D, F-H, and J-L) antibodies tested in competitive ELISA on E1-OVA (55) (A-D), E2-OVA (65) (E-H), or E3-OVA (72) (I-L) coated plates. The inhibition pattern of each antibody was not influenced by ovalbumin antigen used in the assay.

FIG. 13 shows the solution affinity analysis of MDMA binding by the 178F 4H5 mAb Fab fragment. FIG. 13A shows the recording of increasing concentrations of anti-ecstasy 178F 4H5 mAb Fab fragment that were injected over E1-OVA (55) conjugate immobilized on a sensor chip and the initial binding rate (sensogram slope) at 15 s after injection was recorded. To generate a calibration curve, a nonlinear regression plot of the initial binding rate was plotted using a four-parameter fit. FIG. 13B shows the MDMA binding of the anti-ecstasy 178F 4H5 mAb Fab fragment at a fixed concentration of 64 nM, incubated with varying concentrations of MDMA, and allowed to reach equilibrium. The amount of free Fab fragment in solution was determined from the calibration curve and plotted against MDMA concentrations using BIAevaluation software 3.2.1.

FIG. 14 depicts a comparison of the anti-ecstasy 178F 4H5 mAb Fab fragment affinities to ecstasy-class drugs and amphetamines.

FIG. 15 depicts the performance of 178F 4C12 mAb (A), 178F 4H5 mAb (B), 178H 2A7 mAb (C), 178H 4B1 mAb (D), 178K 1F4 mAb (E), 178J 2E11 mAb (F), 178K 1B2 mAb (G), 178K 2B7 mAb (H), 178K 3C8 mAb (I), 178K 4E11 mAb (J), and 178K 5B11 mAb (K) in an EMIT assay.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

It should be understood for any recitation of a method, composition, device, or system that “comprises” certain steps, ingredients, or features, that in certain alternative variations, it is also contemplated that such a method, composition, device, or system may also “consist essentially of” the enumerated steps, ingredients, or features, so that any other steps, ingredients, or features that would materially alter the basic and novel characteristics of the invention are excluded therefrom.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

### A. Definitions

Throughout this description and in the appended claims, the following definitions are to be understood.

As used herein, the terms “ecstasy-class,” “ecstasy class,” and “entactogen” are used to refer to a class of compounds, which includes, without limitation, 3,4-methylenedioxymethamphetamine (MDMA), 3,4 methylenedixoy amphetamine (MDA), N-ethyl-3,4-methylenedioxyamphetamine (MDE), methylenedioxyethylamphetamine (MDEA), N-methyl-1-(3,4-methylenedioxyphenyl)-2-butanamine (MBDB), 1-(3,4-methylenedioxyphenyl)-2-butanamine (BDB), and 3,4-methylenedioxy-N-propylamphetamine (MDPA). As will be appreciated by those of ordinary skill in the art, the ecstasy class is a constantly growing class of drugs, in that drug designers continue to synthesize new, unique compounds which, by virtue of their structure and/or psychedelic properties, fall within the ecstasy class. Accordingly, “ecstasy-class” and “ecstasy class”, as used herein, include compounds which have been synthesized, as well as those which have yet to be synthesized.

The term “immunogen” refers to any substance capable of eliciting an immune response in an organism.

The term “conjugate” refers to any substance formed from the joining together of two parts. Representative conjugates in accordance with the present invention include those formed by the joining together of a small molecule and a large molecule, such as a protein. The term “conjugate” subsumes the term “immunogen.”

The term “hapten” refers to a portion of an immunogen that is typically low in molecular weight, which does not by itself stimulate antibody development.

The phrase “activated hapten” refers to a hapten that has been provided with an available reaction site—for example, by the attachment of a linking group carrying a reactive moiety—that can be used to connect the hapten to a carrier, immunogen, label, tracer, or other moiety.

The term “linking group” (or “linker”) refers to a chemical moiety that is used to connect a hapten to a macromolecular carrier, immunogen, label, tracer, or other moiety. The use of a linking group may or may not be advantageous or needed, depending on the specific hapten and carrier and desired specificity of antibody. Suitable linkers include straight, branched, saturated or unsaturated carbon chains, which may incorporate one or more heteroatoms—that is, atoms other than carbon (e.g., oxygen, nitrogen, sulfur, etc.)—within the chain or substituted onto and/or at a terminus thereof.

The phrases “carrier” and “macromolecular carrier” refer to high molecular weight substances that can be coupled to haptens to form immunogens. Suitable macromolecular carriers include but are not limited to proteins, glycoproteins, polymers, polysaccharides, polypeptides, and nucleic acids that are recognized as foreign and thereby elicit an immunologic response from a host.

The term “polypeptide” refers to any compound formed by the linkage of two or more amino acids via an amide bond. Representative polypeptides include polymers of α-amino acids in which the α-amino group of each non-terminal amino acid residue is linked to the α-carboxyl group of an adjacent residue in a linear chain. High molecular weight polypeptides are referred to as “proteins.”

The term “label” refers to a member of a signal producing system. The label is capable of being detected directly or is detectable through a specific binding reaction that produces a detectable signal. For example, a label may be an identifying tag that can be attached to a carrier substance or molecule to detect an analyte. The labels generally are radioisotopic, luminescent, particulate, or enzymic. The label can be a poly(amino acid), or protein, or non-poly(amino acid), isotopic or non-isotopic, usually non-isotopic, and can be a catalyst, such as an enzyme (e.g., β-galactosidase, peroxidase, etc.), a polynucleotide coding for a catalyst, promoter, dye, fluorescent molecule (e.g., rhodamine, fluorescein isothiocyanate or FITC, etc.), chemiluminescent molecule (e.g., dioxetanes, luciferin, etc.), coenzyme, enzyme substrate, radioactive group (e.g., 125I), a protein-binding partner (e.g., biotin), a small organic molecule, amplifiable polynucleotide sequence, a particle such as latex or carbon particle, metal sol, crystallite, liposome, cell, etc., which may or may not be further labeled with a dye, catalyst or other detectable group, and the like.

The term “non-poly(amino acid) labels” refers to those labels that are not proteins such as enzymes. A non-poly(amino acid) label may be a member of a signal producing system. The non-poly(amino acid) label is capable of being detected directly or is detectable through a specific binding reaction that produces a detectable signal. The non-poly(amino acid) labels generally are radioisotopic, luminescent, particulate, polynucleotidic, or the like. More particularly, the label can be isotopic or non-isotopic, usually non-isotopic, and can be a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, and the like.

The signal producing system may have one or more components, at least one component being the label. The signal producing system generates a signal that relates to the presence of an entactogen in a sample. The signal producing system includes all of the reagents required to produce a measurable signal. Other components of the signal producing system may be included in a developer solution and can include substrates, enhancers, activators, chemiluminescent compounds, cofactors, inhibitors, scavengers, metal ions, specific binding substances required for binding of signal generating substances, and the like. Other components of the signal producing system may be coenzymes, substances that react with enzymic products, other enzymes and catalysts, and the like. The signal producing system provides a signal detectable by external means, by use of electromagnetic radiation, desirably by visual examination. Exemplary signal-producing systems are described in U.S. Pat. No. 5,508,178 (Rose, et al.), the relevant disclosure of which is incorporated herein by reference.

The term “immunogenic carrier” refers to a group which, when conjugated to a hapten and injected into a mammal, will induce an immune response and elicit the production of antibodies that bind to the hapten. Haptens are compounds capable of binding specifically to corresponding antibodies, but do not themselves act as immunogens (or antigens) for preparation of the antibodies. Antibodies that recognize a hapten can be prepared against compounds comprised of the hapten linked to an immunogenic (or antigenic) carrier. Immunogenic carriers are also referred to as antigenic carriers. Typical immunogenic carriers include, without limitation, poly(amino acids), polysaccharides, nucleic acids and particles (biologic and synthetic materials). A wide variety of such carriers are disclosed in Davalian, et al., U.S. Pat. No. 5,089,390, column 4, line 57 to column 5, line 5, incorporated herein by reference. Immunogenic carriers include proteins such as, for example, albumins, serum proteins, e.g., globulins, ocular lens proteins and lipoproteins, and so forth. Illustrative proteins include bovine serum albumin (BSA), keyhole limpet hemocyanin (KLH), egg ovalbumin (OVA), bovine gamma-globulin (BGG), bovine thyroglobulin (BTG), glucose-6-phosphate dehydrogenase (G6PDH), and the like.

The term “antibody” (abbreviated “Ab”) refers to a specific protein capable of binding an immunogen or portion thereof. An antibody may be produced in response to an immunogen, which may have been introduced into a host (e.g., an animal or a human) by injection. Alternatively, an antibody may be produced via hybridoma, phage display, transgenic mice, and CRISPR/Cas9 technologies. The generic term “antibody” subsumes polyclonal antibodies, monoclonal antibodies, and antibody fragments. Monoclonal and polyclonal antibodies (abbreviated “mAb” and “pAb,” respectively) generally include the various classes and isotypes, including IgA, IgD, IgE, IgG1, IgG2A, IgG2B, IgG3, IgG4, IgM, and the like. Antibody fragments may include Fab, scFv, F(ab′) 2, Fab′, and the like. “Antibody” may be used in either a therapeutic or a diagnostic capacity.

The term “analyte” refers to any substance, or group of substances, the presence or amount of which is to be determined. As used herein, the term “analyte” subsumes the term “antigen,” which refers to any compound that can bind to an antibody. Furthermore, as used herein, the term “analyte” refers to all manner of chemical substances including but not limited to: conjugates; immunogens; drugs; drug derivatives; hormones; proteins; antigens; oligonucleotides; and the like. Representative ecstasy drug analytes include but are not limited to MDA, MDMA, MDEA, MDPA, BDB, MBDB, and the like.

The term “derivative” refers to a chemical compound made from a parent compound by one or more chemical reactions.

The phrase “detecting an analyte” refers to any quantitative, semi-quantitative, or qualitative method, as well as to all other methods for determining an analyte in general, and an ecstasy drug in particular. For example, a method that merely detects the presence or absence of an ecstasy drug in a sample lies within the scope of the present invention, as do methods that provide data as to the amount or concentration of the drug in the sample. The terms “detecting,” “determining,” “identifying,” and the like are used synonymously herein, and all lie within the scope of the present invention.

The phrase “reagent kit” or term “kit” refers to an assembly of materials that are used in performing an assay. The reagents can be provided in packaged combination in the same or in separate containers, depending on their cross-reactivities and stabilities, and in liquid or in lyophilized form. The amounts and proportions of reagents provided in the kit can be selected so as to provide optimum results for a particular application. A reagent kit embodying features of the present invention comprises antibodies specific for ecstasy drugs, conjugates of ecstasy-class compounds, and/or enzymes or proteins necessary for detecting the presence and quantity of the antibody or ecstasy drug in a sample. The kit may further comprise calibration and control materials. The reagents may remain in liquid form or may be lyophilized.

The phrase “calibration and control materials” refers to any standard or reference material containing a known amount of an analyte to be measured. A sample suspected of containing an analyte and the corresponding calibration material are assayed under similar conditions. The concentration of analyte is calculated by comparing the results obtained for the unknown specimen with the results obtained for the standard. This is commonly done by constructing a calibration curve.

The phrase “alkyl group” The term “alkyl” (alone or in combination with another term(s)) refers to a saturated hydrocarbon chain of 1 to about 12 carbon atoms in length, such as, but not limited to, methyl, ethyl, propyl, butyl, pentyl, hexyl, heptyl, octyl, decyl, and so forth. The alkyl group may be straight-chain, branched-chain. “Alkyl” is intended to embrace all structural isomeric forms of an alkyl group, cyclic, or acyclic. For example, as used herein, propyl encompasses both n-propyl and isopropyl; butyl encompasses n-butyl, sec-butyl, isobutyl and tert-butyl; pentyl encompasses n¬-pentyl, tert-pentyl, neopentyl, isopentyl, sec-pentyl and 3-pentyl. Further, as used herein, “Me” refers to methyl, “Et” refers to ethyl, “Pr” refers to propyl, “i-Pr” refers to isopropyl, “Bu” refers to butyl, “t-Bu” refers to tert-butyl, “iBu” refers to isobutyl, “Pn” refers to pentyl, and “NPn” refers to neopentyl.

The phrase “optionally substituted” refers to the optional attachment of one or more substituents onto an alkyl group.

The term “sample” refers to a composition to be tested for the presence of an ecstasy-class compound. The sample may be organic or inorganic, biological (e.g., a “biosample”), non-biological, or environmental. Examples of a biological or biosample include, but are not limited to, urine, whole blood, plasma, serum, lymph, mucus, expressed breast milk, semen, stool, sputum, cerebral spinal fluid, tears, hair, saliva, cells, tissues, an organ, and/or a biopsy.

The terms “identical,” “sequence identity,” or “percent identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity for purposes of the present disclosure is determined using the BLAST algorithm, described in Altschul, S. F. & al. (1990) J. Mol. Biol. 215:403-10, which is incorporated herein by reference. The BLAST algorithm is publicly available through software provided by the National Center for Biotechnology Information (at the web address www.ncbi.nlm.nih.gov).

### B. Ecstasy-Class Analogs, Immunogens, and Conjugates

Compounds, such as ecstasy-class analogs, which can be used for preparing immunogens, conjugates, and antibodies useful in immunoassays for the determination of ecstasy-class compounds, are provided herein. Immunogens and conjugates formed from the ecstasy-class compounds are also provided herein. The ecstasy-class analogs are represented by compounds, or salts thereof, where the compounds are a combination of a 2-amino-methylenedioxyphenyl (MDP) derivative and a moiety capable of bonding, either directly or indirectly, with an immunogenic carrier, a detectable label, or a solid capture vehicle. It is contemplated herein that ecstasy-class analogs include haptens and activated haptens.

In any embodiment, such ecstasy-class analog, immunogen, and/or conjugate may correspond in structure to a Formula (I):

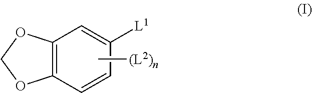

wherein
   L¹ is

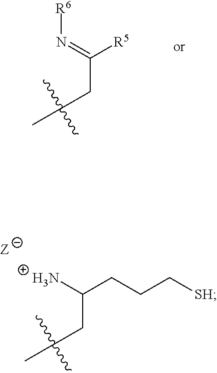

R⁵ is hydrogen or an alkyl;
R⁶ is hydrogen, an alkyl, or —(X⁴)—(Y⁴)ₕ;
   X⁴ is —SH, —NH₂, —COOH, —CONH—, —O(CH₂)(CO)(NH)(C₂H₄)(NH)(CO)(CH₂)Br, —O(CH₂)(CO)(NH)(C₂H₄)(NH)(CO)(CH₂)S—;
   Y⁴ is an immunogenic carrier or a label;
h is zero or 1;
a is zero or 1;
L² is —(CH₂)ᵢ—(X⁵)(Y⁵)ⱼ;
   X⁵ is —SH, —NH₂, —COOH, or —S(CH₂)(CO)NH—,
   Y⁵ is an immunogenic carrier or a label;
   i is 1 to 12;
   j is zero or 1; and
   Z is Cl, (CF₃)CO₂, F, or Br.

In a further embodiment, E4-ecstasy-class analogs, immunogens, and/or conjugates encompassed by Formula (I) may correspond in structure to a Formula (Ic):

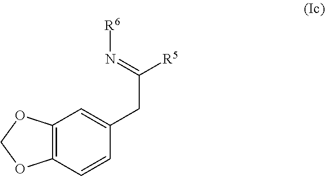

wherein
   R⁵ is hydrogen or an alkyl;
   R⁶ is hydrogen, an alkyl, or —(X⁴)—(Y⁴)ₕ;
      X⁴ is —SH, —NH₂, —COOH, —CONH—, —O(CH₂)(CO)(NH)(C₂H₄)(NH)(CO)(CH₂)Br, —O(CH₂)(CO)(NH)(C₂H₄)(NH)(CO)(CH₂)S—;
   Y⁴ is an immunogenic carrier or a label; and
   h is zero or 1.

In any embodiment, R⁵ may be hydrogen, C₁-C₁₂-alkyl, C₁-C₁₀-alkyl, C₁-C₈-alkyl, C₁-C₆-alkyl, C₁-C₄-alkyl, C₁-C₃-alkyl, C₁-C₂-alkyl or methyl. For example, R⁵ may be C₁-C₆-alkyl, C₁-C₃-alkyl, C₁-C₂-alkyl, or methyl.

Additionally or alternatively, R⁶ may be —(X⁴)—(Y⁴)ₕ. X⁴ may be —O(CH₂)(CO)(NH)(C₂H₄)(NH)(CO)(CH₂)Br or —O(CH₂)(CO)(NH)(C₂H₄)(NH)(CO) (CH₂)S—, and h may be zero or 1. For example, X⁴ may be —O(CH₂) (CO)(NH)(C₂H₄)(NH)(CO)(CH₂)Br and h may be zero. Alternatively, X⁴ may be —O(CH₂)(CO)(NH)(C₂H₄)(NH)(CO)(CH₂)S— and h may be 1.

In any embodiment, R⁵ may be C₁-C₆-alkyl, R⁶ may be —(X⁴)—(Y⁴)ₕ. X⁴ may be —O(CH₂)(CO)(NH)(C₂H₄)(NH)(CO)(CH₂)Br, and h may be zero. In another embodiment, R⁵ may be C₁-C₆-alkyl, R⁶ may be —(X⁴)—(Y⁴)ₕ. X⁴ may be —O(CH₂)(CO)(NH)(C₂H₄)(NH)(CO)(CH₂)S— and h may be 1, and Y⁴ may be keyhole limpet hemocyanin (KLH), bovine serum albumin (BSA), bovine thyroglobulin (BTG), egg ovalbumin (OVA), bovine gamma globulin (BGG), or glucose-6-phosphate dehydrogenase (G6PDH).

In a further embodiment, E6-ecstasy-class analogs, immunogens, and/or conjugates encompassed by Formula (I) may correspond in structure to a Formula (Id):

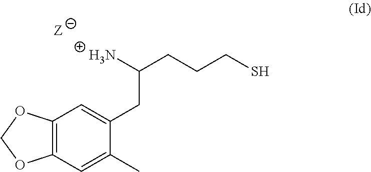

wherein Z is Cl, (CF₃)CO₂, F, or Br. In particular, Z may be Cl.

In any embodiment, such ecstasy-class analog, immunogen, and/or conjugate may correspond in structure to a Formula (I):

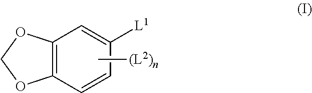

wherein
   L¹ is

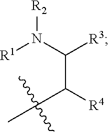

R¹ is hydrogen, an alkyl, or —CO(CF₃);
R² is hydrogen, an alkyl, or —(CH₂)_{b}—(X¹)—(Y¹)_{c};
   X¹ is —SH, —NH₂, —COOH, —CONH—, —S(CH₂)(CO)NH—,
CO(NH)(C₂H₄)S(CH₂)(CO)NH—, —CO(NH)(C₂H₄)SH, or

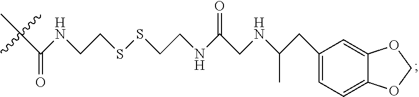 [SMILES: *C(=O)NCCSSCCNC(=O)CNC(C)Cc1ccc2c(c1)OCO2]

Y¹ is an immunogenic carrier or a label;
b is 1 to 10;
c is zero or 1;
R³ is hydrogen, alkyl, or —(CH₂)_{d}—(X²)—(Y²)ₑ;
   X² is —SH, —NH₂, —COOH, —CONH—, —S(CH₂)(CO)NH—,
   —CO(NH)(C₂H₄)S(CH₂)(CO)NH—, or

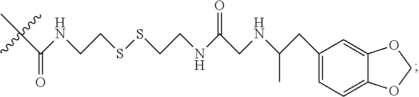 [SMILES: *C(=O)NCCSSCCNC(=O)CNC(C)Cc1ccc2c(c1)OCO2]

Y² is an immunogenic carrier or a label;
d is 2 to 12;
e is zero or 1;
R⁴ is hydrogen or —(CH₂)_{f}(X³)—(Y³)_{g};
   X³ is —SH, —NH₂, —COOH, —CONH—, —S(CH₂)(CO)NH—, —CO(NH)(C₂H₄)S(CH₂)(CO)NH—, or

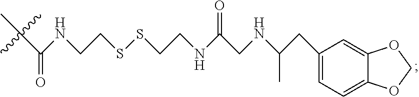 [SMILES: *C(=O)NCCSSCCNC(=O)CNC(C)Cc1ccc2c(c1)OCO2]

Y³ is an immunogenic carrier or a label;
f is 1 to 12;
g is zero or 1;
a is zero or 1;
L² is —(CH₂)ᵢ—(X⁵)(Y⁵)ⱼ;
   X⁵ is —SH, —NH₂, —COOH, or —S(CH₂)(CO)NH—,
   Y⁵ is an immunogenic carrier or a label;
   i is 1 to 12; and
   j is zero or 1.

In any embodiment, when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ may be hydrogen.

Additionally or alternatively, when a is zero, R¹ is hydrogen or methyl, R² is —(CH₂)_{b}—(X¹)—(Y¹)_{c}. X¹ is —CONH—, c is 1, Y¹ is an immunogenic carrier or a label, R³ is methyl, R⁴ is hydrogen, then b may be 5-10.

Additionally or alternatively, when a is zero, R² is —(CH₂)_{b}—(X¹)—(Y¹)_{c}, b is 3 or 4, X¹ is —COOH, c is zero, R³ is methyl, R⁴ is hydrogen, then R¹ may be hydrogen.

Additionally or alternatively, when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b may be 5-10.

Additionally or alternatively, at least one of R¹, R², and R³ is neither hydrogen nor alkyl.

In further embodiments, E1-, E2-, and E3-ecstasy-class analogs, immunogens, and/or conjugates encompassed by Formula (I) may correspond in structure to a Formula (Ia):

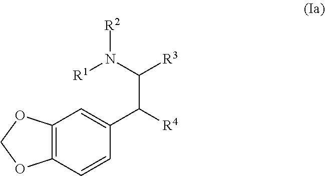

wherein
   R¹ is hydrogen, an alkyl, or —CO(CF₃);
   R² is hydrogen, an alkyl, or —(CH₂)_{b}—(X¹)—(Y¹)_{c};
      X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or

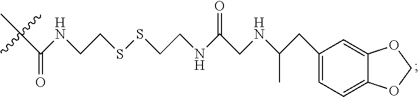 [SMILES: *C(=O)NCCSSCCNC(=O)CNC(C)Cc1ccc2c(c1)OCO2]

Y¹ is an immunogenic carrier or a label;
b is 1 to 10;
c is zero or 1;
R³ is hydrogen, alkyl, or —(CH₂)_{d}—(X²)—(Y²)ₑ;
   X² is —SH, —NH₂, —COOH, —CONH—, —S(CH₂)(CO)NH—, —CO(NH)(C₂H₄)S(CH₂)(CO)NH—, or

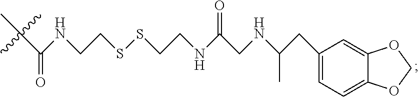 [SMILES: *C(=O)NCCSSCCNC(=O)CNC(C)Cc1ccc2c(c1)OCO2]

Y² is an immunogenic carrier or a label;
d is 2 to 12;
e is zero or 1;
R⁴ is hydrogen or —(CH₂)_{f}(X³)—(Y³)_{g}; X³ is —SH, —NH₂, —COOH, —CONH—, —S(CH₂)(CO)NH—, —CO(NH)(C₂H₄)S(CH₂)(CO)NH—, or

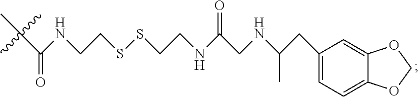 [SMILES: *C(=O)NCCSSCCNC(=O)CNC(C)Cc1ccc2c(c1)OCO2]

Y³ is an immunogenic carrier or a label;
f is 1 to 12; and
g is zero or 1.

In any embodiment, R¹ may be hydrogen, C₁₋₆ alkyl, or —CO(CF₃). In further embodiments, R¹ may be hydrogen or —CO(CF₃). Additionally or alternatively, R² may be hydrogen, C₁₋₆ alkyl, or —(CH₂)_{b}—(X¹)—(Y¹)_{c} and b may be 1 to 5. In any embodiment, b may be 1 to 5, 1 to 4, 1 to 3, 1 to 2, such as, 1 or 4. X¹ may be —COOH, —CONH—, —CO(NH)(C₂H₄)S(CH₂)(CO)NH—, —CO(NH)(C₂H₄)SH, or

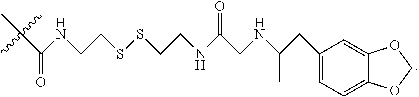 [SMILES: *C(=O)NCCSSCCNC(=O)CNC(C)Cc1ccc2c(c1)OCO2]

In further embodiments, X¹ may be —COOH, —CO(NH)(C₂H₄)SH, or

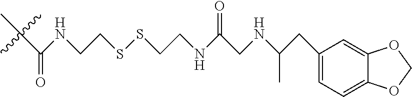 [SMILES: *C(=O)NCCSSCCNC(=O)CNC(C)Cc1ccc2c(c1)OCO2]

or X¹ may be —CONH— or —CO(NH)(C₂H₄)S(CH₂)(CO)NH—.

Additionally or alternatively, R³ may be C₁₋₆ alkyl or —(CH₂)_{d}—(X²)—(Y²)ₑ. In further embodiments, R³ may be C₁-C₃-alkyl, C₁-C₂-alkyl, or methyl. Additionally or alternatively, R⁴ is hydrogen. For example, R³ may be C₁₋₆ alkyl, such as methyl, and R⁴ may be hydrogen.

In some embodiments, R¹ may be hydrogen, or —CO(CF₃); R² may be —(CH₂)_{b}—(X¹)—(Y¹)_{c}, b may be 1 to 5, and X¹ may be —COOH, —CONH—, —CO(NH)(C₂H₄)S(CH₂)(CO)NH—, —CO(NH)(C₂H₄)SH, or

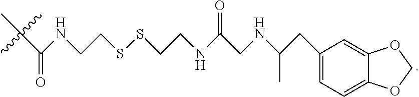 [SMILES: *C(=O)NCCSSCCNC(=O)CNC(C)Cc1ccc2c(c1)OCO2]

In further embodiments, X¹ may be —COOH, —CO(NH)(C₂H₄)SH, or

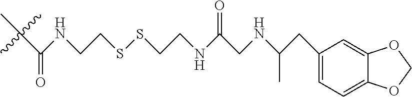 [SMILES: *C(=O)NCCSSCCNC(=O)CNC(C)Cc1ccc2c(c1)OCO2]

and c may be zero. Alternatively, X¹ may be —CONH— or —CO(NH)(C₂H₄)S(CH₂)(CO)NH— and c may be 1. Additionally or alternatively, R³ may be methyl and R⁴ may be hydrogen.

In any embodiment, R¹ and R⁴ may each be hydrogen, R³ may be methyl, and R² may be —(CH₂)_{b}—(X¹)—(Y¹)_{c}, where b may be 1, c is 0, and X¹ may be

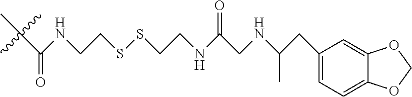 [SMILES: *C(=O)NCCSSCCNC(=O)CNC(C)Cc1ccc2c(c1)OCO2]

or —CO(NH)(C₂H₄)SH.

In another embodiment, R¹ and R⁴ may each be hydrogen, R³ may be methyl, and R² may be —(CH₂)_{b}—(X¹)—(Y¹)_{c}, b may be 1, X¹ may be —CO(NH)(C₂H₄)S(CH₂)(CO)NH—, and c is 1. Alternatively, R¹ and R⁴ may each be hydrogen, R³ may be methyl, and R² may be —(CH₂)_{b}—(X¹)—(Y¹)_{c}, b may be 4, X¹ may be —CONH—, and c may be 1. In either instance, Y¹ may be keyhole limpet hemocyanin (KLH), bovine serum albumin (BSA), bovine thyroglobulin (BTG), egg ovalbumin (OVA), bovine gamma globulin (BGG), or glucose-6-phosphate dehydrogenase (G6PDH).

In any embodiment, R¹ may be hydrogen and R² may be hydrogen or C₁₋₆ alkyl. Additionally or alternatively, R³ may be —(CH₂)_{d}—(X²)—(Y²), and R⁴ may be hydrogen. X² is —S(CH₂)(CO)NH—, and e is 1. In any embodiment, d may be 2 to 12, 2 to 10, 2 to 8, 2 to 6, 2 to 4, or 2 to 3, such as 2, 3, or 4. X² may be —S(CH₂)(CO)NH—, and e may be zero or 1. Alternatively, R³ may be C₁₋₆ alkyl and R⁴ may be —(CH₂)_{f}(X³)—(Y³) g.

In any embodiment, R¹ may be hydrogen, R² may be hydrogen, R³ may be —(CH₂)_{d}—(X²)—(Y²)ₑ, R⁴ may be hydrogen, d may be 2 to 6, X² may be —S(CH₂)(CO)NH—, and e may be 1. In such instance, Y² may be keyhole limpet hemocyanin (KLH), bovine serum albumin (BSA), bovine thyroglobulin (BTG), egg ovalbumin (OVA), bovine gamma globulin (BGG), or glucose-6-phosphate dehydrogenase (G6PDH).

In a further embodiment, E5-ecstasy-class analogs, immunogens, and/or conjugates encompassed by Formula (I) may correspond in structure to a Formula (Ib):

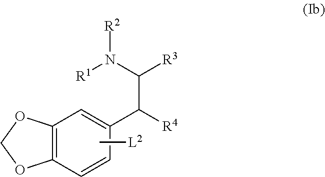

wherein
   R¹ is hydrogen, an alkyl, or —CO(CF₃);
   R² is hydrogen, an alkyl, or —(CH₂)_{b}—(X¹)—(Y¹)_{c};
      X¹ is —SH, —NH₂, —COOH, —CONH—, —S(CH₂)(CO)NH—, —CO(NH)(C₂H₄)S(CH₂)(CO)NH—, or

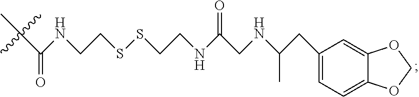 [SMILES: *C(=O)NCCSSCCNC(=O)CNC(C)Cc1ccc2c(c1)OCO2]

Y¹ is an immunogenic carrier or a label;
b is 1 to 10;
c is zero or 1;
R³ is hydrogen, alkyl, or —(CH₂)_{d}—(X²)—(Y²)ₑ; X² is —SH, —NH₂, —COOH, —CONH—, —S(CH₂)(CO)NH—, —CO(NH)(C₂H₄)S(CH₂)(CO)NH—, or

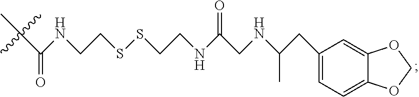 [SMILES: *C(=O)NCCSSCCNC(=O)CNC(C)Cc1ccc2c(c1)OCO2]

Y² is an immunogenic carrier or a label;
d is 2 to 12;
e is zero or 1;
R⁴ is hydrogen or —(CH₂)_{f}(X³)—(Y³)_{g};
   X³ is —SH, —NH₂, —COOH, —CONH—, —S(CH₂)(CO)NH—, —CO(NH)(C₂H₄)S(CH₂)(CO)NH—, or

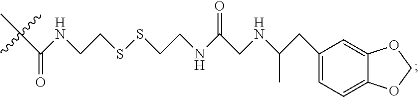 [SMILES: *C(=O)NCCSSCCNC(=O)CNC(C)Cc1ccc2c(c1)OCO2]

Y³ is an immunogenic carrier or a label;
f is 1 to 12; and
g is zero or 1;
L² is —(CH₂)ᵢ—(X⁵)—(Y⁵)ⱼ;
   X⁵ is —SH, —NH₂, —COOH, or —S(CH₂)(CO)NH—,
   Y⁵ is an immunogenic carrier or a label;
   i is 1 to 12; and
   j is zero or 1.

In any embodiment, each of R¹, R², R³, and R⁴ independently may be hydrogen, C₁-C₁₂-alkyl, C₁-C₁₀-alkyl, C₁-C₈-alkyl, C₁-C₆-alkyl, C₁-C₄-alkyl, C₁-C₃-alkyl, C₁-C₂-alkyl or methyl. For example, each of R¹, R², R³, and R⁴ may be hydrogen, C₁-C₆-alkyl, C₁-C₃-alkyl, C₁-C₂-alkyl, or methyl. In a further embodiment, each of R¹, R², and R⁴ may be hydrogen and R³ may be C₁-C₆-alkyl, C₁-C₃-alkyl, C₁-C₂-alkyl, or methyl.

Additionally or alternatively, X⁵ may be —SH or —S(CH₂)(CO)NH, i may be 1 to 12, to 1 to 10, 1 to 7, 1 to 5, 1 to 4, or 1 to 3, such as 1, 2, 3, 4, or 5, and j may be zero or 1. For example, X⁵ may be —SH and j may be zero. Alternatively, X⁵ may be —S(CH₂)(CO)NH and j may be 1.

In any embodiment, each of R¹, R², and R⁴ may be hydrogen, R³ may be C₁-C₆-alkyl, X⁵ may be —SH, i may be 1 to 5, and j may be zero. Alternatively, each of R¹, R², and R⁴ may be hydrogen, R³ may be C₁-C₆-alkyl, X⁵ may be —S(CH₂)(CO)NH, i may be 1 to 5, and j may be 1.

In any embodiment, R¹, R², and R⁴ may be hydrogen, R³ may be methyl, a may be 1, L² may be —(CH₂)ᵢ—(X⁵)(Y⁵)_{j′} where i is 4, X⁵ may be —SH, and g is zero. Alternatively, R¹, R², and R⁴ may be hydrogen, R³ may be methyl, a may be 1, L² may be —(CH₂)ᵢ—(X⁵)—(Y⁵)_{j′} where i is 4, X⁵ may be —S(CH₂)(CO)NH—, g is 1, and Y⁵ is an immunogenic carrier which may be keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

In any embodiment, an alkyl group may be C₁-C₁₂-alkyl, C₁-C₁₀-alkyl, C₁-C₈-alkyl, C₁-C₆-alkyl, C₁-C₄-alkyl, C₁-C₃-alkyl, C₁-C₂-alkyl or methyl. For example, the alkyl group may be C₁-C₆-alkyl, C₁-C₃-alkyl, C₁-C₂-alkyl, or methyl. The alkyl group may be straight-chained, branched, or cyclic. In various aspects, the alkyl group may be methyl.

As stated above, Formula I encompasses ecstasy-class analogs, such as haptens and activated haptens as well as immunogens and conjugates formed from the ecstasy-class analogs. In such instances when Formula I represents an immunogen or conjugate, each of Y¹ to Y⁵ independently can be an immunogenic carrier, a label, or an enzyme.

Suitable immunogenic carriers include, but are not limited to, a protein, a polypeptide, a polysaccharide, a nucleic acid, and a particle (e.g., biologic and synthetic materials). A wide variety of such carriers are disclosed U.S. Pat. No. 5,089,390, incorporated herein by reference. Examples of suitable proteins include, but are not limited to, albumins, serum proteins, e.g., globulins, ocular lens proteins and lipoproteins, and so forth. Nonlimiting examples of proteins include the keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH). A protein may be attached to a linking group by means of an amine group on the protein.

In any embodiment, a label may be radioisotopic, luminescent, particulate or enzymic. The label can be a poly(amino acid), or protein, or non-poly(amino acid), isotopic or non-isotopic, and can be a catalyst, such as an enzyme (e.g., β-galactosidase, peroxidase, etc.), a polynucleotide coding for a catalyst, promoter, dye, fluorescent molecule (e.g., rhodamine, fluorescein isothiocyanate or FITC, etc.), chemiluminescent molecule (e.g., dioxetanes, luciferin, etc.), coenzyme, enzyme substrate, radioactive group (e.g., ¹²⁵I), a protein-binding partner, biotin or another small organic molecule, amplifiable polynucleotide sequence, a particle such as latex or carbon particle, metal sol, crystallite, liposome, cell, etc., which may or may not be further labeled with a dye, catalyst or other detectable group, and the like.

Examples of suitable enzymes include, but are not limited to, glucose-6-phosphate dehydrogenase (G6PDH), an alkaline phosphatase, and horseradish peroxidase (HRP).

Nonlimiting examples of compounds (e.g., ecstasy-analogs, haptens, activated haptens) corresponding in structure to Formulas (I), (Ia), (Ib), (Ic), and/or (Id) are shown below in Table 1.

TABLE 1
Compounds

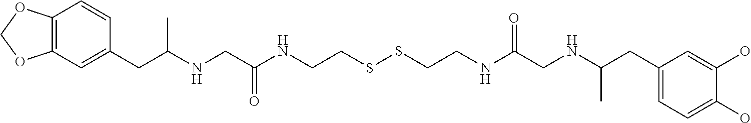 [SMILES: CC(Cc1ccc(O)c(O)c1)NCC(=O)NCCSSCCNC(=O)CNC(C)Cc1ccc2c(c1)OCO2]

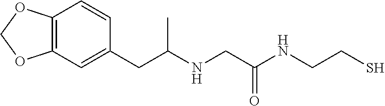 [SMILES: CC(Cc1ccc2c(c1)OCO2)NCC(=O)NCCS]

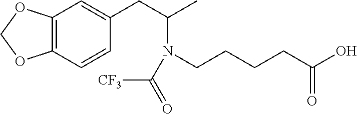 [SMILES: CC(Cc1ccc2c(c1)OCO2)N(CCCCC(=O)O)C(=O)C(F)(F)F]

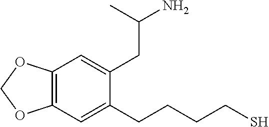 [SMILES: CC(N)Cc1cc2c(cc1CCCCS)OCO2]

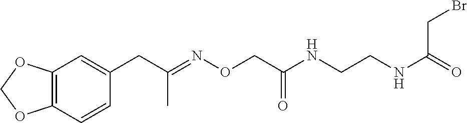 [SMILES: C/C(Cc1ccc2c(c1)OCO2)=N\OCC(=O)NCCNC(=O)CBr]

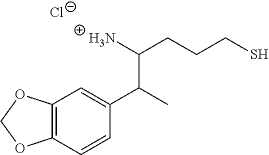 [SMILES: [Cl-] ; CC(c1ccc2c(c1)OCO2)C([NH3+])CCCS]

Nonlimiting examples of conjugates and/or immunogens corresponding in structure to Formulas (I), (Ia), (Ib), (Ic), and/or (Id) are shown below in Table 2.

TABLE 2
Immunogens and/or Conjugates

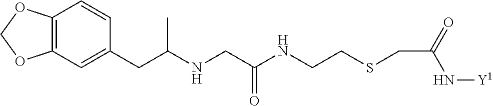

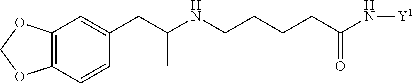

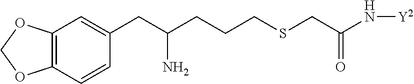

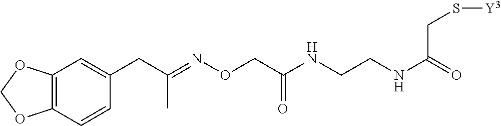

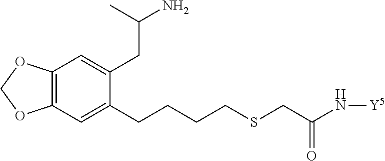

Y¹, Y², Y⁴, and Y⁵ each=keyhole limpet hemocyanin (KLH), bovine serum albumin (BSA), bovine thyroglobulin (BTG), egg ovalbumin, bovine gamma globulin (BGG), or glucose-6-phosphate dehydrogenase (G6PDH).

### C. Synthesis

The syntheses of representative examples of the above compounds (e.g., ecstasy analogs, haptens, activated haptens, immunogens, and conjugates) are discussed herein by way of illustration and not limitation. Other synthetic procedures will be suggested to those skilled in the art in view of the disclosure herein. Other compounds within the scope of the present invention may be prepared using suitable variants of the reagents employed below. The reaction temperatures and time are those customary for the type of reactions conducted and should be evident to those skilled in the art.

For immunogen and conjugate syntheses, a protein may be attached to a linking group by means of an amine group or a thiol group on the protein. The formulas and compounds described herein show the nitrogen atom of the amine group of the protein or the sulfur atom of the thiol group of the protein. In general, functional groups suitable for attaching a compound as described herein (e.g., hapten, activated hapten) to an immunogenic carrier (e.g., protein, enzyme) or label are usually an activated ester or alkylating agent when the amino acid(s) that are to be conjugated on the enzyme have amino or hydroxyl groups and are usually alkylating agents or the like when the amino acid(s) that are to be conjugated on the immunogenic carrier comprise a sulfur atom such as, e.g., a cysteine. A large number of suitable functional groups are available for attaching to amino groups and alcohols such as activated esters including imidic esters, sulfonic esters and phosphate esters, activated nitrites, aldehydes, ketones, alkylating agents and the like. Conjugation of haptens to proteins using these and other attaching groups are well known in the art and are described in reviews such as for example, Maggio, E. T. “Enzyme-Immunoassay” (CRC Press, Boca Raton, Fla., 1980), Chapter 4, which contains an assortment of conjugation techniques; pages 81-88 of which are incorporated herein by reference.

Following reaction of the immunogenic carrier (e.g., protein, enzyme) with a compound such as discussed above to form a conjugate, the product is then optionally purified as may be required. The purification and characterization of poly(amino acid)-hapten conjugates has been described in detail Maggio, et al.; “Enzyme-immunoassay” (CRC Press, Boca Raton, Fla., 1980), Chapter 4, pages 86-88 of which are incorporated herein by reference. For example, the protein-hapten conjugate can be purified, for example, by dialysis against aqueous/organic and aqueous solutions or by gel filtration chromatography on a support such as Sephadex®, and the like.

As mentioned above, the conjugation can involve binding of a hapten to a free thiol group present on an amino acid side chain of the enzyme (e.g. cysteine). Such conjugation involves alkylation of the thiol sulfur atom by treatment with an electrophilic compound such as an alpha- or beta-unsaturated amide, ketone, ester, or the like, or an alkylating agent such as a reactive halide, e.g., bromide, or sulfonate or the like or reaction with an active disulfide such as a 2-nitro-4-carboxyphenyl disulfide. Specific examples by way of illustration and not limitation include alpha-bromoamides, maleimides, vinyl sulfones, alpha-iodoketones, and the like.

Conjugation reactions with proteins or enzymes can be affected by a number of factors. These include, but are not confined to, pH, temperature, buffer, ionic strength, substances which may protect the enzyme active site, amount and type of cosolvent, reaction time, and activation chemistry. A range of pH values from about 5.0 to about 9.5 can usually be used for conjugation reactions. These reactions are generally carried out at about 0° C. to about 40° C., preferably about 4° C. to about 20° C.

A number of buffers and salts, both alone and in combination, can be used for such reactions. These include Tris, bicarbonate, phosphate, pyrophosphate, ethylenediaminetetraacetic acid (EDTA), KCl, NaCl, and many others. The active site may be protected by substrates (i.e., glucose-6-phosphate and compounds that react reversibly with lysine (i.e., pyridoxal) to reduce deactivation of the enzyme during conjugation.

Co-solvents which may enhance hapten solubility include, but are not limited to, dimethylformamide, carbitol, dimethyl sulfoxide, 1-methyl-2-pyrrolidinone, and 1,3-dimethyl-3,4,5,6-tetrahydro 2 (1H)-pyrimidinone. These may be useful as about 1 to about 30% of the reaction volume. Reactions can vary from about 15 min to many days, depending on the activation chemistry. Carboxylic compounds may be activated to form esters with N-hydroxysuccinimide or its sulfo-analog, or to mixed anhydrides through reaction with carbitol chloroformate or t-butylchloroformate, or may be coupled directly using carbodiimides such as EDC. For reaction with cysteine thiols on the enzyme, the hapten should contain a good leaving group such as I, Br, or tosyl; alternatively, the hapten can contain a thiol, preferably activated with 2,2′ dithiodipyridine, 5,5′ dithiobis(2-nitrobenzoic acid) (DTNB), dithioerythritol (DTE), and the like.

Another method of conjugation, described in Rowley, G. L., D. Leung, and P. Singh (U.S. Pat. No. 4,220,722) involves modification of the immunogenic carrier (e.g., protein, enzyme) with bromoacetyl containing reactants; the bromo groups are subsequently reacted with thiol-containing haptens. The reaction of the immunogenic carrier (e.g., protein, enzyme) with bromoacetyl modifier, and the bromoacetyl enzyme with the thiolated hapten, are subject to the same reaction condition variables described above.

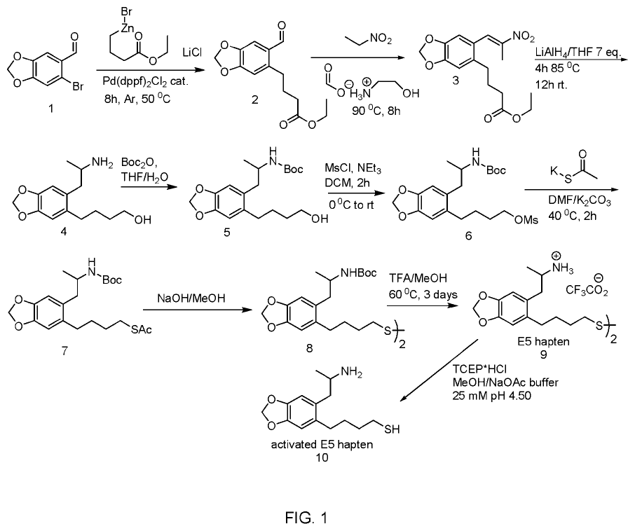

Referring to FIG. 1, the synthesis of activated E5 hapten (10) may begin with combining commercially available starting material 1 with a catalyst, such as Pd (dppf) 2Cl₂, and a salt (e.g., LiCl) along with an ether (e.g., tetrahydrofuran (THF)) and an ethoxy-4-oxobutylzinc bromide-THE solution to give compound 2. Compound 2 can be suspended in a mixture of formic acid:ethanolamine along with nitroethane and reacted to produce nitro compound 3. The nitro group of compound 3 may be reduced using a suitable reducing agent such as, for example, lithium aluminum hydride, aluminum borohydride, etc., in an organic medium such as, for example, an aromatic hydrocarbon, an ether (e.g., ethyl ether, THF, etc.), a formamide (e.g., dimethylformamide), and so forth and combinations thereof, e.g., ether/toluene to produce intermediate compound 4. Compound 4 in solution (e.g., in THF) is combined with triethylamine (NEt₃) and di-tert-butyl dicarbonate (BoczO) in a THF solution and reacted to produce compound 5. Compound 5 may be suspended in a suitable solvent (e.g., dichloromethane (DCM)) and NEt₃ may be added therein followed by mesyl chloride (MsCl) to give compound 6. Compound 6 may be dissolved in THF and solution of potassium thioacetate in N,N-Dimethylformamide in (DMF) and reacted to form compound 7. Compound 7 may be suspended in an alcohol (e.g., methanol (MeOH)) then a base, such as sodium hydroxide (NaOH), and subsequent dimerization of the by-product takes place to generate compound 8. An alcohol (e.g., MeOH) and a suitable acid (e.g., trifluoroacetic acid (TFA)) can be added to compound 8 and reacted to yield compound 9. Compound 9 may be suspended in an alcohol (e.g., MeOH) and a buffer, such as sodium acetate (NaOAc), as well as tris(2-carboxyethyl) phosphine hydrochloride (TCEP·HCl) can be added to the mixture to yield activated E5 hapten (10).

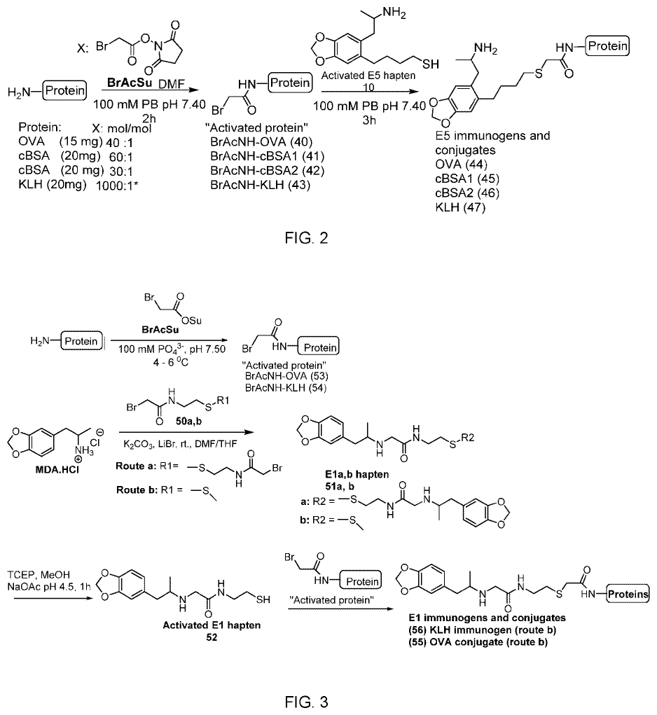

Referring to FIG. 2, the amine group of a protein such as, for example, OVA, cBSA, KLH, and the like is treated with succinimidylbromo-acetate (BrAcSu) to introduce the bromo-acetamide functional group for thiol modification giving activated protein BrAcNH-OVA (40), activated protein BrAcNH-cBSA1 (41), activated protein BrAcNH-cBSA1 (42) and activated protein BrAcNH-KLH (43). Reaction of activated E5 hapten (10) (see FIG. 2) with activated protein BrAcNH-OVA (40), activated protein BrAcNH-cBSA1 (41), activated protein BrAcNH-cBSA1 (42) and activated protein BrAcNH-KLH (43) gives the desired conjugates [e.g., E5-OVA conjugate (44), E5-cBSA1 conjugate (45), E5-cBSA2 conjugate (46), and E5-KLH immunogen (47)]. Reaction conditions include, for example, a buffer solution at pH of about 7 to 9, about 7.5 to 8.5, about 8. Such buffer solutions include, for example, phosphate or borate buffer etc., and combinations thereof. The resulting immunogens may be purified by appropriate purification techniques such as, for example, dialysis or column chromatography, e.g., Sephadex®, etc., and the like using a suitable eluent, e.g., phosphate buffer, etc. The attachment of a protein to the linking moiety of the molecule can be through the amino group on a protein, where the nitrogen of the amino group may be the nitrogen of the linking group depicted above.

Referring to FIG. 3, to prepare an E1-hapten (compound 51a,b), MDA·HCl may be suspended in a solvent (e.g., DMF), K₂CO₃, and LiBr and a solution of linker 50a (“route a”) or linker 50b (“route b”) in an ether (e.g., THF) is added and reacted to produce an E1-hapten compound 51a,b. Routes a and b use different linkers but they can generate the same activated hapten by following the same coupling chemistry. E1-haptens (51a,b) can be activated by suspending the E1-hapten (51a,b) in an alcohol (e.g., MeOH) and a buffer (e.g., NaOAc) as well as TCEP·HCl can be added to the mixture to yield activated E1 hapten (compound 52). FIG. 3 further provides syntheses of the desired immunogens and conjugates from activated E1 hapten (52). Activated protein BrAcNH-OVA (53) and activated protein BrAcNH-KLH (54) can be prepared as described above (see FIG. 2). Reaction of E1 hapten with activated protein BrAcNH-OVA (53) and activated protein BrAcNH-KLH (54) gives the desired conjugates or immunogens [e.g., E1-OVA conjugate (55) and E1-KLH immunogen (56)]. Reaction conditions include, for example, a buffer solution at pH of about 7 to 9, about 7.5 to 8.5, about 8. Such buffer solutions include, for example, phosphate buffer, e.g., a dihydrogen phosphate, a hydrogen phosphate, etc., and combinations thereof. The resulting conjugates or immunogens may be purified by appropriate purification techniques such as, for example, dialysis or column chromatography, e.g., Sephadex®, etc., and the like using a suitable eluent, e.g., phosphate buffer, etc.

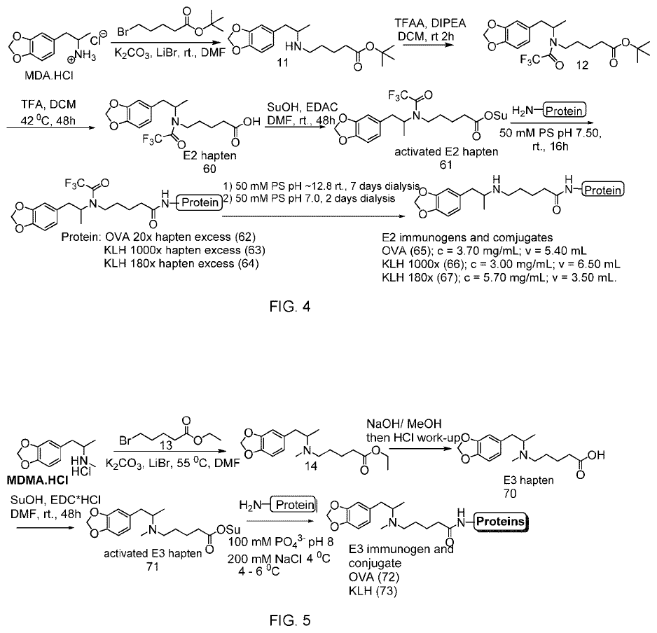

Referring to FIG. 4, to prepare an E2-hapten (compound 60), MDA·HCl may be dissolved in acetonitrile and K₂CO₃ may be added. Linker, tert-butyl-5-bromovalerate, is added and reacted with the mixture to yield compound 11. Compound 11 may be suspended in a solvent (e.g., DCM) and N,N-diisopropylethylamine (DIPEA) and TFAA may be added and reacted to produce compound 12. Compound 12 may be suspended a solvent (e.g., DCM) and an acid (e.g., TFA) may be added and reacted to give E2-hapten (60). The E2-hapten (60) may be activated by suspending the E2-hapten (60) in a solvent (e.g., DMF) and adding N-hydroxysuccinimide (SuOH) and N-(3-dimethylaminopropyl)-N′-ethylcarbodiimide hydrochloride (EDC·HCl) to give E2-activated hapten (61). Each protein (OVA, KLH) can be suspended in a buffer (e.g., phosphate buffer or tris buffer) and then a solution of activated E2 hapten (61) in a solvent (e.g., DMF) may be added to each protein solution to produce intermediate E2-conjugates or E2-immunogens 62, 63, and 64. Intermediate E2-conjugates (62, 63, and 64) may then be dialyzed with a buffer (e.g., phosphate or tris buffer) to deprotect the trifluoroacetate group and produce the desired conjugates or immunogens (e.g., E2-OVA (65), E2-KLH 100× (66), and E2-KLH 180× (67)) via filtering.

Referring to FIG. 5, to prepare an E3-hapten (compound 70), MDA·HCl may be dissolved in solvent (e.g., DMF) and K₂CO₃ may be added. Linker 13 (ethyl-5-bromovalerate) and LiBr may be added and reacted with the mixture to yield compound 14. Compound 14 may be mixed with an alcohol (e.g., MeOH) and a base (e.g., NaOH) and an acid (e.g., HCl) may be added to the acidify the mixture and yield hapten compound 70. E3-hapten (70) may be activated by suspending the E3-hapten (70) in a solvent (e.g., DMF) and adding SuOH and EDC·HCl to give activated E3 hapten (71). Each protein (OVA, KLH) can be suspended in a buffer (e.g., phosphate or tris buffer) and then a solution of E3-activated hapten (71) in a solvent (e.g., DMF) may be added to each protein solution to produce the desired conjugates or immunogens (e.g., E3-OVA (72), E3-KLH (73)). The resulting conjugates or immunogens may be purified by appropriate purification techniques such as, for example, dialysis or column chromatography, e.g., Sephadex®, etc., and the like using a suitable eluent, e.g., phosphate buffer, etc.

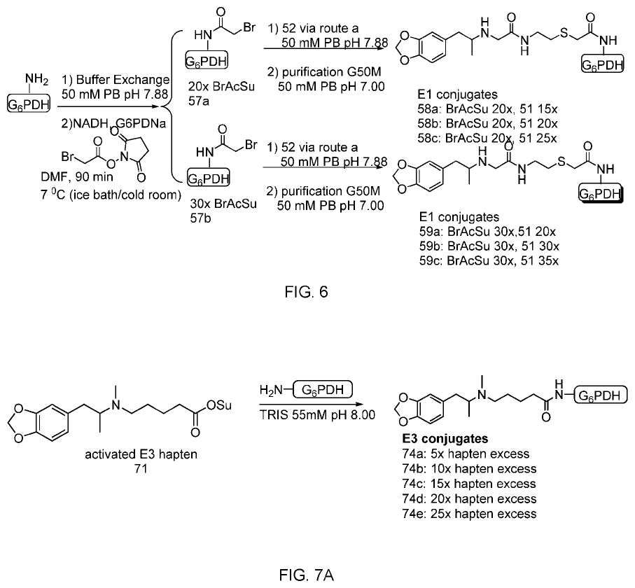

Referring to FIG. 6, to prepare an E1-G6PDH enzyme conjugate with an activated E1-hapten (compound 52) (e.g., formed via route a in Scheme 3), a native G6PDH enzyme is buffer exchanged with a buffer (e.g., phosphate or tris buffer) and can be further diluted with the buffer. Glucose-6-phosphate di-sodium salt (G6PDNa₂) and nicotinamide adenine dinucleotide (β-NADH) are added to the enzyme solution. To the enzyme solution, a solution of BrAcSu (e.g., in DMF) is added. The resulting reaction mixtures may be further buffer exchanged to produce G6PDH activated enzymes (e.g., activated enzyme 57a, activated enzyme 57b). An activated E1-hapten (52) may be added to the G6PDH activated enzymes to produce the desired E1-G6PDH conjugates (e.g., 58a, 58b, 58c, 59a, 59b, 59c). The resulting E1-conjugates may be purified by appropriate purification techniques such as, for example, dialysis or column chromatography, e.g., Sephadex®, etc., and the like using a suitable eluent, e.g., phosphate buffer, etc.

Referring to FIG. 7A, to prepare an E3-G6PDH enzyme conjugate with an activated E3 hapten (71), a G6PDH enzyme is buffer exchanged with a buffer (e.g., Tris buffer) and can be further diluted with the buffer. G6PDNa₂ and β-NADH are added to the enzyme solution. Activated E3 hapten (71) (e.g., in DMF solution), for example, in varying volumes, may be added to the enzyme solution to produce the desired E3-G6PDH conjugates (e.g., 74a, 74b, 74c, 74d, 74e). The resulting conjugates may be purified by appropriate purification techniques such as, for example, dialysis or column chromatography, e.g., Sephadex®, etc., and the like using a suitable eluent, e.g., phosphate buffer, etc.

Referring to FIG. 7B, to prepare an E2-G6PDH enzyme conjugate with an activated E2 hapten (61), a G6PDH enzyme is buffer exchanged with a buffer (e.g., Tris 2) and can be further diluted with the buffer. G6PDNa₂ and β-NADH are added to the enzyme solution. Activated E2 hapten (61) (e.g., in DMF solution), for example, in varying volumes, may be added to the G6PDH enzyme solution followed by a buffer exchange with a buffer (e.g., phosphate or tris buffer) to produce the desired E2-G6PDH conjugates (e.g., 68a, 68b, 68c, 68d, 68e). The resulting conjugates may be purified by appropriate purification techniques such as, for example, dialysis or column chromatography, e.g., Sephadex®, etc., and the like using a suitable eluent, e.g., phosphate buffer, etc.

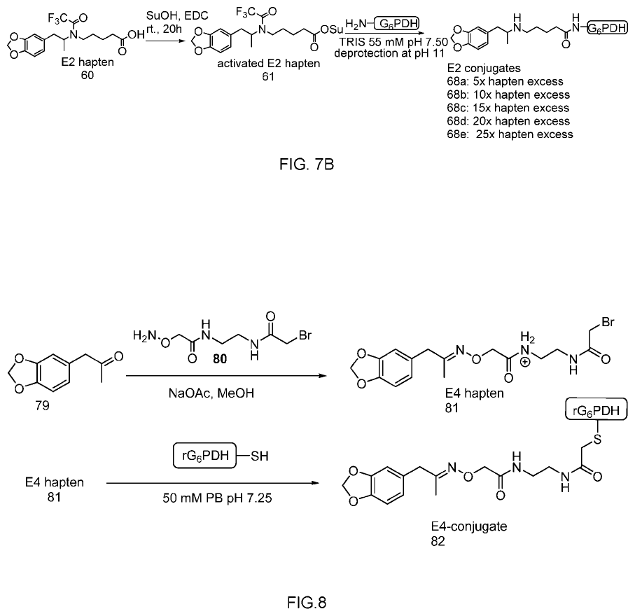

Referring to FIG. 8, to prepare an E4 hapten (compound 81), piperonyl methyl ketone (PMK) (79) may be suspended in an alcohol (e.g. MeOH) and NaOAc and linker 80 (aminooxyaminobromoacetate) may be added and reacted to yield an E4 hapten (81). To prepare an E4-G6PDH enzyme conjugate with an E4 hapten (81), a G6PDH enzyme is reduced and buffer exchanged with a buffer (e.g., phosphate or tris buffer). The E4 hapten (81) (e.g., in DMF solution), for example, in varying volumes, may be added to the G6PDH enzyme solution to produce the desired E4-G6PDH conjugate (82). The resulting conjugate may be purified by appropriate purification techniques such as, for example, dialysis or column chromatography, e.g., Sephadex®, etc., and the like using a suitable eluent, e.g., phosphate buffer, etc.

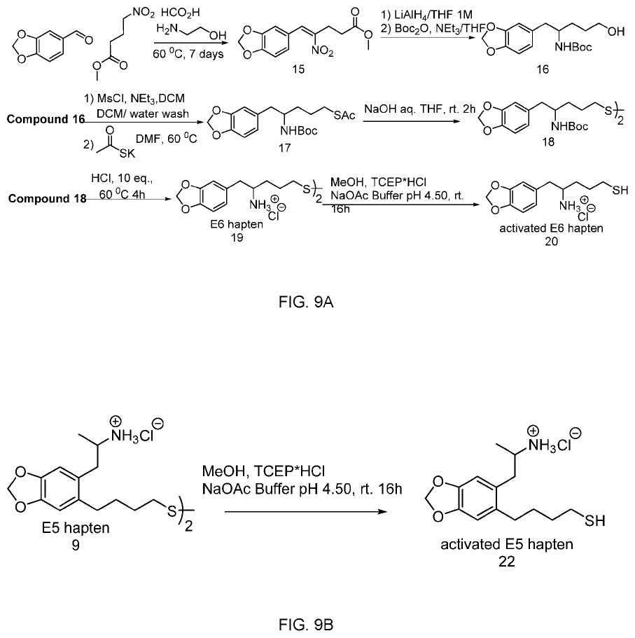

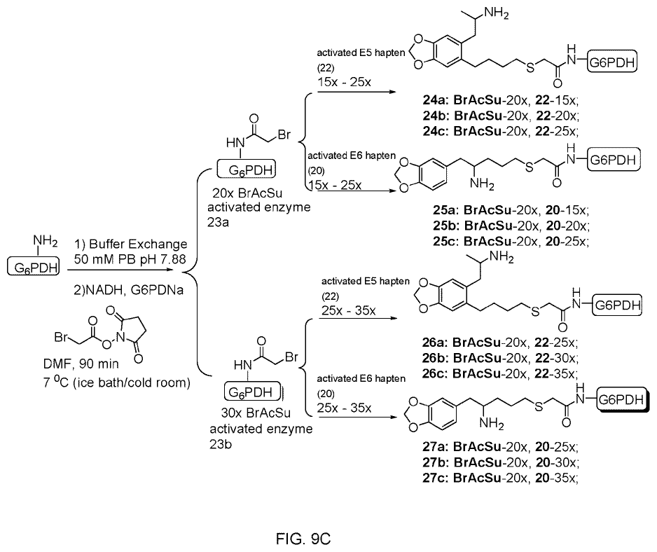

Referring to FIG. 9A, to prepare an E6 hapten (compound 19), PMK and methyl-4-nitrobutirate may be added to a mixture of formic acid:aminoethanol and reacted. Ethyl acetate may be added to the reaction mixture to give compound 15. Compound 15 may be dissolved in an ether (e.g., THF) and LiAlH₄/THF may be added to the reaction mixture and reacted. NEt₃ and a BoczO-THE solution may be added to reaction mixture to yield compound 16. Compound 16 may be dissolved in a solvent (e.g., DCM) and NEt₃ and MsCl may be added to the reaction mixture. The reaction mixture may be concentrated via evaporation and an intermediate may be formed and suspended in an ether (e.g., THF) to which thioacetate (e.g., in DMF) and K₂CO₃ are added to give compound 17. Compound 17 may be dissolved in an alcohol (e.g., MeOH) and a base (e.g., NaOH) may be added to produce compound 18. Compound 18 may be dissolved in an alcohol (e.g., MeOH) and an acid (e.g., HCl) may be added to produce an E6-hapten (19). The E6 hapten (19) may be activated by dissolving the E6 hapten (19) in an alcohol (e.g., MeOH) to which a buffer (e.g., NaOAc) and TCEP·HCl are added and reacted to yield activated E6 hapten (20).

Referring to FIG. 9B, for preparation of an activated E5 hapten (compound 22), E5 hapten (9) maybe be dissolved in an alcohol (e.g., MeOH) to which a buffer (e.g., NaOAc) and TCEP·HCl are added and reacted at pH 4.5 for 16 h at room temperature to yield an activated E5 hapten (22).

Referring to FIG. 9C, to prepare an E5- or E6-G6PDH enzyme conjugate with an activated E5 hapten (22) or an activated E6 hapten (20), a G6PDH enzyme is buffer exchanged with a buffer (e.g., PBS) and can be further diluted with the buffer. G6PDNa₂ and β-NADH are added to the enzyme solution. BrAcSu (e.g., in DMF) can be added to the enzyme solution, for example, in varying volumes, to produce activated enzyme BrAcSu-G6PDH (e.g., 23a, 23b). E6 hapten (20) and E5 hapten (22), for example, in varying volumes, may be added to the activated enzyme BrAcSu-G6PDH to produce the desired E5-G6PDH conjugates (e.g., 24a, 24b, 24c, 26a, 26b, 26c) or E6-G6PDH conjugates (e.g., 25a, 25b, 25c, 27a, s7b, s7c). The resulting E5- or E6-conjugates may be purified by appropriate purification techniques such as, for example, dialysis or column chromatography, e.g., Sephadex®, etc., and the like using a suitable eluent, e.g., phosphate buffer, etc.

### D. Antibodies

Antibodies raised against the compounds described above and useful in immunoassays for the determination of ecstasy-class compounds are provided herein.

In any embodiment, the antibodies may be raised against a compound of Formula (I), including any one of Formula (Ia), Formula (Ib), Formula (Ic), and/or Formula (Id), wherein an immunogenic carrier is present (e.g., Y¹, Y², Y³, Y⁴, and/or Y⁵ may be KLH, BSA, BTG, OVA, BGG, or G6PDH). In particular, an antibody may be raised against an E1 immunogen corresponding in structure to:

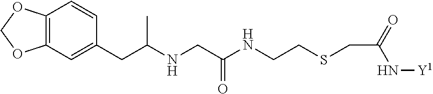

wherein Y¹ may be KLH, BSA, BTG, OVA, BGG, or G6PDH. The antibodies described herein may preferentially bind to an ecstasy-class compound. Suitable ecstasy-class compounds include, but are not limited to, 3,4-methylenedioxymethamphetamine (MDMA) and/or 3,4-methylenedioxy amphetamine (MDA). The preferential binding of the antibody to an ecstasy-class compound may be measured by any suitable means, including but not limited to a dissociation constant (K_{D}) of the antibody for the ecstasy-class compound. For example, an antibody disclosed herein may have a dissociation constant (K_{D}) for ecstasy-class compounds (e.g., MDA or MDMA) of about 10⁻⁵ M or less, about 10⁻⁶ M or less, about 10⁻⁷ M or less, about 10⁻⁸ M or less, about 10⁻⁹ M or less, about 10⁻¹⁰ M or less, or about 10⁻¹¹ M or less.

Additionally or alternatively, an antibody disclosed herein may have a dissociation constant (K_{D}) for ecstasy-class compounds in a range of about 10⁻⁵ M to about 10⁻¹¹ M, about 10⁻⁵ M to about 10⁻¹⁰ M, about 10⁻⁵ M to about 10⁻⁹ M, about 10⁻⁵⁷ M to about 10⁻⁸ M, about 10⁻⁵ M to about 10⁻⁷ M, about 10⁻⁵ M to about 10⁻⁶ M, about 10⁻⁶ M to about 10⁻¹¹ M, about 10⁻⁶ M to about 10⁻¹⁰ M, about 10⁻⁶ M to about 10⁻⁹ M, about 10⁻⁶ M to about 10⁻⁸ M about 10⁻⁶ M to about 10⁻⁷ M, about 10⁻⁷ M to about 10⁻¹¹ M, about 10⁻⁷ M to about 10⁻¹⁰ M, about 10⁻⁷ M to about 10⁻⁹ M, about 10⁻⁷ M to about 10⁻⁸ M, about 10⁻⁸ M to about 10⁻¹¹ M, about 10⁻⁸ M to about 10⁻¹⁰ M, and about 10⁻⁸ M to about 10⁻⁹ M. A particular K_{D} may be determined by the identity of the ecstasy-class compound bound by the antibody. For example, the antibody may bind to MDA with a K_{D} of about 10⁻⁷ to about 10⁻¹⁰ M. Additionally or alternatively, the antibody may bind to MDMA with a K_{D} of about 10⁻⁷ M to about 10⁻¹⁰ M.

Additionally or alternatively, the preferential binding of an antibody may be measured by an amount, e.g., a percentage, of an ecstasy-class compound recovered from a sample, such as a sample from an immunoassay. In any example, an antibody may recover at least about 70%, at least about 75%, at least about 80%, at least about 85%, at least about 90%, at least about 95%, or at least about 99% of an ecstasy-class compound.

In particular, an antibody may recover at least about 80% of MDA from a sample in an immunoassay. Additionally or alternatively, the antibody may recover at least about 80% MDMA from a sample in an immunoassay. The immunoassay is not particularly limited, and may be an enzyme multiplied immunoassay technique (EMIT), an enzyme-linked immunosorbent assay (ELISA), a radioimmunoassay (RIA), an enzyme channeling immunoassay (ECIA), a fluorescence polarization immunoassay (FPIA), an enzyme modulate mediated immunoassay (EMMIA), a substrate labeled fluorescence immunoassay (SLFIA), a combined enzyme donor immunoassay (CEDIA), a particle enhanced turbidimetric inhibition immunoassay (PETINIA), a particle enhanced turbidimetric immunoassay (PETIA), a sol particle immunoassay (SPIA), a disperse dye immunoassay (DIA), a metalloimmunoassay (MIA), an enzyme membrane immunoassays (EMIA), or a luminoimmunoassays (LIA). In a specific embodiment, the immunoassay may be an EMIT immunoassay.

In any embodiment, an antibody may not cross-react or exhibit limited cross-reactivity to certain compounds, such as non-ecstasy-class compounds. Here, cross-reactivity is measured by the amount of an undesired compound in a sample bound to the antibody. Nonlimiting examples of an undesired compound (e.g., a non-ecstasy-class compound) include, but are not limited to, amphetamine, methamphetamine, and 4-hydroxy-3-methoxymethamphetamime. An antibody that binds to about 50% of the undesired compound in a sample is interpreted as having a cross-reactivity of about 50%. The antibodies disclosed herein may have a cross-reactivity of less than about 60%, less than about 55%, less than about 50%, less than about 45%, less than about 40%, less than about 35%, less than about 30%, less than about 25%, less than about 20%, less than about 15%, less than about 10%, less than about 5%, or less than about 1%. In particular, an antibody may have a cross-reactivity of less than about 50% with each of amphetamine, methamphetamine, and 4-hydroxy-3-methoxymethamphetamine.

### E. Advantageous Antibody Properties

Advantageously, it has been surprisingly discovered that antibodies described herein may bind with equal affinity, or almost equal affinity (e.g., having the same or similar K_{D}) to two or more ecstasy-class compounds, such as both MDA and MDMA, but may also have low cross-reactivity (e.g., a lower affinity) with non-ecstasy-class compounds (e.g., amphetamine and methamphetamine). For example, an antibody may be highly specific for MDA and MDMA with a binding affinity for both compounds in a nanomolar range but may have low cross-reactivity with (e.g., a lower affinity for) non-ecstasy-class compounds (e.g., amphetamine and methamphetamine) with a binding affinity within or near the micromolar range. Thus, the antibodies described herein, for example, antibodies raised against a compound of Formula (I), such as E1-KLH (56), may distinguish ecstasy-class compounds (e.g., MDA and MDMA) from non-ecstasy-class compounds (e.g., amphetamine and methamphetamine) in the same sample or different samples, even though the two groups of compounds (ecstasy-class and non-ecstasy-class compounds) are structurally similar.

In any embodiment, the antibody may include a heavy chain comprising a leader sequence, three complementarity determining regions (termed “HC-CDR 1,” “HC-CDR 2,” and “HC-CDR 3” as used herein) and linking regions flanking the CDRs (collectively referred to as the “variable heavy chain”), and a constant region at the C-terminal end of the polypeptide sequence. The term “full heavy chain” refers to the portion of the antibody comprising the heavy variable chain and the heavy chain constant region. The regions of the heavy chain may be in the order of a leader sequence, a first linker region, HC-CDR 1, a second linker region, HC-CDR 2, a third linker region, HC-CDR 3, a fourth linker region, and a constant region.

The antibody may further comprise a light chain comprising a leader sequence, three CDRs (termed “LC-CDR 1,” “LC-CDR 2,” and “LC-CDR 3” as used herein), and linker regions flanking the CDRs (collectively referred to as the “variable light chain”), and a constant region at the C-terminal end of the polypeptide sequence. The term “full light chain” refers to the portion of the antibody comprising the light variable chain and the light chain constant region. The regions of the light chain may be in the order of a leader sequence, a first linker region, LC-CDR 1, a second linker region, LC-CDR 2, a third linker region, LC-CDR 3, a fourth linker region, and a constant region.

Consensus, specific, and alternate amino acid sequences for the various regions of the light chain and heavy chain are shown in Table 3 below. A “specific sequence” is a sequence in which all amino acids (or nucleotides) are identified and are encompassed within the consensus sequence, which is a sequence having one or more positions of variable identity (indicated with an “X”). The specific sequences described below represent the mature peptide or portion of the antibody. The specific sequences are not further modified when incorporated within a larger polypeptide and/or protein (e.g., a full-length antibody). An “alternate sequence” is a sequence that is not encompassed within the consensus sequence. In some instances, multiple specific sequences may be encompassed within a single consensus sequence.

TABLE 3

| | SEQ ID | |
|---|---|---|
| Region | NO: | Sequence |
| HC leader (consensus) | 1 | MXXXXXLXXXXXXXXXXXX |
| HC leader (specific) | 2 | MNFGLSLIFLALILKGVQC |
| HC leader (specific) | 3 | MRVLILLWLFTAFPGILS |
| HC-CDR 1 | 4 | XXVMS |
| (consensus) | | |
| HC-CDR 1 (specific) | 5 | NYVMS |
| HC-CDR 1 (specific) | 6 | RFVMS |
| HC-CDR 1 (specific) | 7 | SYVMS |
| HC-CDR 1 (specific) | 8 | RNVMS |
| HC-CDR 1 (specific) | 9 | RDVMS |
| HC-CDR 1 (specific) | 10 | AYVMS |
| HC-CDR 1 (alternate) | 11 | SGYGWH |
| HC-CDR 2 | 12 | TIXXXGXXXXYXXXXVKG |
| (consensus) | | |
| HC-CDR 2 (specific) | 13 | TINSGGSYTYYPDTVKG |
| HC-CDR 2 (specific) | 14 | TISSGGIHTYYLDSVKG |
| HC-CDR 2 (specific) | 15 | TISYGGGHYTYYPDSVKG |
| HC-CDR 2 (specific) | 16 | TISSGGVHTYYLDSVKG |
| HC-CDR 2 (specific) | 17 | TINSGGRYAYYPDSVKG |
| HC-CDR 2 (specific) | 18 | TINSVGRYTYYTDSVKG |
| HC-CDR 2 (alternate) | 19 | YINYSGHIELNPSLKD |
| HC-CDR 2 (specific) | 20 | TINSGGRFTYYPDSVKG |
| HC-CDR 2 (specific) | 21 | TISSNGIYIYYTDSVKG |
| HC-CDR 2 (specific) | 22 | TINSGGRYTYYPDSVKG |
| HC-CDR 2 (specific) | 23 | TISRGGSYIYYPDSVKG |
| HC-CDR 3 | 24 | GNXXDX |
| (consensus) | | |
| HC-CDR 3 (specific) | 25 | GNFLDY |
| HC-CDR 3 (specific) | 26 | GNALDY |
| HC-CDR 3 (specific) | 27 | GNFLDY |
| HC-CDR 3 (specific) | 28 | GNALNY |
| HC-CDR 3 (specific) | 29 | GNNLDY |
| HC-CDR 3 (specific) | 30 | GNNLDF |
| HC-CDR 3 (alternate) | 31 | GGGLYSSYGGDY |
| HC-CDR 3 (specific) | 32 | GNNLDS |
| HC-CDR 3 (specific) | 179 | GNYLDY |
| LC leader (specific) | 33 | MRFSAQLLGLLVLWIPGSTA |
| LC-CDR 1 | 34 | RSSKS LLXXX GITYL YX |
| (consensus) | | |
| LC-CDR 1 (specific) | 35 | RSSKS LLNSY GITYL Y |
| LC-CDR 1 (specific) | 36 | RSSKSLLHSNGITYLY |
| LC-CDR 1 (specific) | 37 | RSSKSLLHYTGITYLY |
| LC-CDR 1 (specific) | 38 | RSSKSLLQRNGITYLY |
| LC-CDR 1 (alternate) | 39 | LASQTIGTWLA |
| LC-CDR 1 (specific) | 40 | RSSKSLLQRSGITYLY |
| LC-CDR 1 (specific) | 41 | RSSKSLLHRNGITYLY |
| LC-CDR 1 (specific) | 42 | RSSKSLLQINGITYLY |
| LC-CDR 1 (specific) | 180 | RSSKSLLQRNGITYLH |
| LC-CDR 2 | 43 | XXSNLAS |
| (consensus) | | |
| LC-CDR 2 (specific) | 44 | QMSNLAS |
| LC-CDR 2 (specific) | 45 | RLSNLAS |
| LC-CDR 2 (specific) | 46 | GAIRLAD |
| LC-CDR 2 (specific) | 47 | RMSNLAS |
| LC-CDR 3 | 48 | GQDXELPTX |
| (consensus) | | |
| LC-CDR 3 (specific) | 49 | GQDLELPYT |
| LC-CDR 3 (specific) | 50 | GQDLELPYS |
| LC-CDR 3 (alternate) | 51 | QQLYRSPYA |
| LC-CDR 3 (specific) | 52 | GQDMELPYT |

The antibody may comprise both a variable heavy chain and a variable light chain, wherein the variable heavy chain comprises a first heavy chain CDR (also described as “HC-CDR 1”) as set forth in SEQ ID NO:4 or SEQ ID NO:11, a second heavy chain CDR (also described as “HC-CDR 2”) as set forth in SEQ ID NO: 12 or SEQ ID NO: 19, and a third heavy chain CDR (also described as “HC-CDR 3”) as set forth in SEQ ID NO: 24 or SEQ ID NO: 31 and the variable light chain comprises a first light chain CDR (also described as “LC-CDR 1”) as set forth in SEQ ID NO:34 or SEQ ID NO:39, a second light chain CDR (also described as “LC-CDR 2”) as set forth in SEQ ID NO: 43 or SEQ ID NO: 46, and a third light chain CDR (also described as “LC-CDR 3”) as set forth in SEQ ID NO: 48 or SEQ ID NO: 51. Additionally and alternatively, the heavy chain CDRs of the described antibody include: a HC-CDR 1 comprising an amino acid sequence having at least about 80% sequence identity, at least about 85% sequence identity, at least about 90% sequence identity, or at least about 95% sequence identity with the sequence of SEQ ID NO:5, SEQ ID NO:6, SEQ ID NO:7, SEQ ID NO:8, SEQ ID NO:9, SEQ ID NO:10, or SEQ ID NO:11; a HC-CDR 2 comprising an amino acid sequence having at least 80% sequence identity, at least 85% sequence identity, at least about 90% sequence identity, or at least about 95% sequence identity with the sequence of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; and a HC-CDR 3 comprising an amino acid sequence having at least about 80% sequence identity, at least about 85% sequence identity, at least about 90% sequence identity, or at least about 95% sequence identity with the sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32 or SEQ ID NO:179. In a further embodiment, the heavy chain CDRs of the described antibody may include: a HC-CDR 1 having an amino acid sequence of SEQ ID NO:5, SEQ ID NO:6, SEQ ID NO:7, SEQ ID NO:8, SEQ ID NO:9, SEQ ID NO:10, or SEQ ID NO:11; a HC-CDR 2 having an amino acid sequence of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; and a HC-CDR 3 having an amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:179. In a still further embodiment, the described antibody may comprise a variable heavy chain having an amino acid sequence having at least about 80% sequence identity, at least about 85% sequence identity, at least about 90% sequence identity, or at least about 95% sequence identity with the sequence of SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, or SEQ ID NO:182. Additionally or alternatively, the described antibody comprises a variable heavy chain having an amino acid sequence having a sequence of SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, or SEQ ID NO:182.

In any embodiment, a variable heavy chain of an antibody further comprises a heavy chain leader sequence having at least about 80% sequence identity, at least about 85% sequence identity, at least about 90% sequence identity, or at least about 95% sequence identity with the sequence of SEQ ID NO: 2 or SEQ ID NO: 3. Alternatively, a heavy chain of an antibody further comprises a heavy chain leader sequence having a sequence of SEQ ID NO: 2 or SEQ ID NO: 3.

In any embodiment, a variable heavy chain of an antibody is covalently bound to a heavy chain constant region having at least about 80% sequence identity, at least about 85% sequence identity, at least about 90% sequence identity, or at least about 95% sequence identity with the sequence of SEQ ID NO: 53 to form a full heavy chain. In a further embodiment, a variable heavy chain of an antibody is covalently bound to a heavy chain constant region having the sequence of SEQ ID NO: 53 to form a full heavy chain. Additionally or alternatively, a heavy chain constant region comprises an amino acid sequence having at least about 80% sequence identity, at least about 85% sequence identity, at least about 90% sequence identity, or at least about 95% sequence identity with the sequence of SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65; or SEQ ID NO:181. More specifically, the heavy chain constant region comprises an amino acid sequence having the sequence of SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65; or SEQ ID NO:181.

In any embodiment, a full heavy chain (which includes the leader sequence, three CDRs, each of which are flanked by a linker sequence, and a constant region) comprises an amino acid sequence having at least about 80% sequence identity, at least about 85% sequence identity, at least about 90% sequence identity, or at least about 95% sequence identity with the sequence of SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90 or SEQ ID NO:183. Additionally or alternatively, a full heavy chain comprises an amino acid sequence having the sequence of SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90 or SEQ ID NO:183.

In any embodiment, a light chain CDRs of the described antibody include: a LC-CDR 1 comprising an amino acid sequence having at least 80% sequence identity, at least about 85% sequence identity, at least about 90% sequence identity, or at least about 95% sequence identity with the sequence of SEQ ID NO:35, SEQ ID NO:36, SEQ ID NO:37, SEQ ID NO:38, SEQ ID NO:39, SEQ ID NO:40, SEQ ID NO:41, SEQ ID NO:42 or SEQ ID NO:180; a LC-CDR 2 comprising an amino acid sequence having at least about 80% sequence identity, at least about 85% sequence identity, at least about 90% sequence identity, or at least about 95% sequence identity with the sequence of SEQ ID NO:44, SEQ ID NO:45, SEQ ID NO:46, or SEQ ID NO:47; and a LC-CDR 3 comprising an amino acid sequence having at least about 80% sequence identity, at least about 85% sequence identity, at least about 90% sequence identity, or at least about 95% sequence identity with the sequence of SEQ ID NO:49, SEQ ID NO:50, SEQ ID NO:51, or SEQ ID NO:52. In a further embodiment, a light chain CDRs of the described antibody include: a LC-CDR 1 comprising an amino acid sequence of SEQ ID NO:35, SEQ ID NO:36, SEQ ID NO:37, SEQ ID NO:38, SEQ ID NO:39, SEQ ID NO:40, SEQ ID NO:41, SEQ ID NO:42, or SEQ ID NO:180; a LC-CDR 2 comprising an amino acid sequence of SEQ ID NO:44, SEQ ID NO:45, SEQ ID NO:46, or SEQ ID NO:47; and a LC-CDR 3 comprising an amino acid sequence of SEQ ID NO:49, SEQ ID NO:50, SEQ ID NO:51, or SEQ ID NO:52. In a still further embodiment, the described antibody comprises a variable light chain having an amino acid sequence having at least about 80% sequence identity, at least about 85% sequence identity, at least about 90% sequence identity, or at least about 95% sequence identity with the sequence of SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, or SEQ ID NO:184. Additionally or alternatively, the described antibody comprises a variable light chain having an amino acid sequence having a sequence of SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, or SEQ ID NO:184.

In any embodiment, a variable light chain of an antibody further comprises a light chain leader sequence having at least about 80% sequence identity, at least about 85% sequence identity, at least about 90% sequence identity, or at least about 95% sequence identity with the sequence of SEQ ID NO: 33. In a further embodiment, a light chain of an antibody further comprises a light chain leader sequence with the sequence identity of SEQ ID NO: 33.

In any embodiment, a variable light chain of an antibody is covalently bound to a light chain constant region having at least about 80% sequence identity, at least about 85% sequence identity, at least about 90% sequence identity, or at least about 95% sequence identity with the sequence of SEQ ID NO: 66 to form a full light chain. In a further embodiment, the variable light chain of the antibody may be covalently bound to a light chain constant region having the sequence of SEQ ID NO: 66 to form a full light chain.

In any embodiment, a full light chain (which includes the leader sequence, three CDRs, each of which are flanked by a linker sequence, and a constant region) comprises an amino acid sequence having at least about 80% sequence identity, at least about 85% sequence identity, at least about 90% sequence identity, or at least about 95% sequence identity with a sequence of SEQ ID NO:103, SEQ ID NO:104, SEQ ID NO:105, SEQ ID NO:106 SEQ ID NO:107, SEQ ID NO:108, SEQ ID NO:109, SEQ ID NO:110, SEQ ID NO:111, SEQ ID NO:112, SEQ ID NO:113, SEQ ID NO:114; or SEQ ID NO:185. Additionally or alternatively, a full light chain comprises an amino acid sequence having the sequence of SEQ ID NO:103, SEQ ID NO:104, SEQ ID NO:105, SEQ ID NO:106 SEQ ID NO:107, SEQ ID NO:108, SEQ ID NO:109, SEQ ID NO:110, SEQ ID NO:111, SEQ ID NO:112, SEQ ID NO:113, SEQ ID NO:114; or SEQ ID NO:185.

The full heavy and light chains described above may be joined by disulfide bonds to link any full light chain with any full heavy chain and/or to link any full heavy chain with another full heavy chain. In such manner, the antibody contains two full heavy chains and two full light chains. When a light chain/heavy chain pair is linked to a light chain/heavy chain pair having the same CDRs, the antibody recognizes and binds a single epitope and is a monovalent antibody. Alternatively, when a light chain/heavy chain pair is linked to a light chain/heavy chain pair having different CDRs, the antibody may recognize and bind to two different epitopes and is a polyvalent (or divalent) antibody. Exemplary monoclonal antibodies having specific heavy and light chains may be produced by inoculating an animal (e.g., mice or rabbits) with an embodiment of Formula (I), for example, including an ecstasy-class compound as a hapten, a linker, and an immunogenic carrier. The ecstasy-class compound may be MDMA, and the immunogenic carrier may be KLH. In any embodiment, a compound of Formula (I) used to inoculate an animal (e.g., a mouse) may be E1-KLH (56), an MDA-KLH conjugate having the following structure:

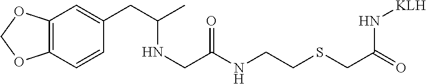

In a method for producing monoclonal antibodies, single splenocytes from the inoculated mice may be fused with myeloma cells to create hybridomas which produced particular embodiments of monoclonal antibodies described herein (shown in Table 4 below). The heavy and light chain amino acid sequences of the exemplified clones are also shown. Where only a single heavy chain and single light are listed, the antibody contains a pair of the combination (e.g., both heavy chain and light chain pairs of the 178F 1A8 mAb comprise SEQ ID NO: 79 as the full heavy chain and SEQ ID NO: 103 as the full light chain).

TABLE 4

| | Heavy Chain | Heavy | Light chain | Light |
|---|---|---|---|---|
| | Amino Acid | Chain DNA | Amino Acid | Chain DNA |
| mAb | SEQ ID NO | SEQ ID NO | SEQ ID NO | SEQ ID NO |
| 178F 1A8 | 79 | 141 | 103 | 167 |
| 178F 4C12 | 80 | 142 | 104 | 168 |
| 178F 4H5 | 81 | 143 | 105 | 169 |
| 178H 2A7 | 82 | 144 | 106 | 170 |
| 178H 4B1 | 83 | 145 | 107 | 171 |
| 178K 1F4 | 85 | 147 | 109 | 173 |
| 178J 2E11 | 86 | 148 | 110 | 174 |
| 178K 1B2 | 84 | 146 | 108 | 172 |
| 178K 2B7 | 87 | 149 | 111 | 175 |
| 178K 3C8 | 88 | 150 | 112 | 176 |
| 178K 4E11 | 89 | 151 | 113 | 177 |
| 178K 5B11 | 90 | 152 | 114 | 178 |
| 178K 1E11 | 183 | 188 | 185 | 190 |

### F. Polynucleotides

The antibodies described above can be prepared by conventional means known in the art. When monoclonal antibodies are desired, the amino acid sequence can be encoded by a polynucleotide (e.g., a DNA sequence) and provided to a cell (e.g., a hybridoma, bacteria, yeast, etc.) which translates the nucleotide sequence to the antibody. The polynucleotides described herein are isolated or purified and may be incorporated into a vector (e.g., a viral vector, a plasmid, etc.) for insertion into a cell (e.g., transformation, transfection, etc.) for subsequent production of the antibody.

In any embodiment, a polynucleotide comprising a DNA sequence encoding any one of the heavy chain or light chain CDRs is provided herein. For example, a polynucleotide may encode one or more of heavy chain CDR 1 (HC-CDR 1), HC-CDR 2, and/or HC-CDR 3. The polynucleotide may have a nucleotide sequence having at least about 80% sequence identity, at least about 85% sequence identity, at least about 90% sequence identity, or at least about 95% sequence identity with a sequence of SEQ ID NO: 191, SEQ ID NO: 192, SEQ ID NO: 193, SEQ ID NO: 194, SEQ ID NO: 195, or SEQ ID NO: 196 for HC-CDR1; a nucleotide sequence having at least about 80%, at least about 85% sequence identity, at least about 90% sequence identity, or at least about 95% sequence identity with a sequence of SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, or SEQ ID NO: 207 for HC-CDR 2; and/or a nucleotide sequence having at least about 80%, at least about 85% sequence identity, at least about 90% sequence identity, or at least about 95% sequence identity with a sequence of SEQ ID NO: 208, SEQ ID NO: 209, SEQ ID NO: 210, SEQ ID NO: 211, SEQ ID NO: 212, SEQ ID NO: 213, SEQ ID NO: 214, or SEQ ID NO: 215 for HC-CDR 3. Additionally or alternatively, a polynucleotide encoding a variable heavy chain as described herein may have a nucleotide sequence of SEQ ID NO: 191, SEQ ID NO: 192, SEQ ID NO: 193, SEQ ID NO: 194, SEQ ID NO: 195, or SEQ ID NO: 196 for HC-CDR 1; a nucleotide sequence of SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, or SEQ ID NO: 207 for HC-CDR 2; and/or a nucleotide sequence of SEQ ID NO: 208, SEQ ID NO: 209, SEQ ID NO: 210, SEQ ID NO: 211, SEQ ID NO: 212, SEQ ID NO: 213, SEQ ID NO: 214, or SEQ ID NO: 215 for HC-CDR 3.

Additionally or alternatively, a polynucleotide may encode one or more of light chain CDR 1 (LC-CDR 1), LC-CDR 2, and/or LC-CDR 3. The polynucleotide may have a nucleotide sequence having at least about 80%, at least about 85% sequence identity, at least about 90% sequence identity, or at least about 95% sequence identity with a sequence of SEQ ID NO: 216, SEQ ID NO: 217, SEQ ID NO: 218, SEQ ID NO: 219, SEQ ID NO: 220, SEQ ID NO: 221, SEQ ID NO: 222, or SEQ ID NO: 223 for LC-CDR 1; a polynucleotide may have a nucleotide sequence having at least about 80%, at least about 85% sequence identity, at least about 90% sequence identity, or at least about 95% sequence identity with a sequence of SEQ ID NO: 224, SEQ ID NO: 225, SEQ ID NO: 226, or SEQ ID NO: 227 for LC-CDR 2; and/or a polynucleotide may have a nucleotide sequence having at least about 80%, at least about 85% sequence identity, at least about 90% sequence identity, or at least about 95% sequence identity with a sequence of SEQ ID NO: 228, SEQ ID NO: 229, SEQ ID NO: 230, or SEQ ID NO: 231 for LC-CDR 3. Additionally or alternatively, a polynucleotide encoding a variable heavy chain as described herein may have a nucleotide sequence of SEQ ID NO: 216, SEQ ID NO: 217, SEQ ID NO: 218, SEQ ID NO: 219, SEQ ID NO: 220, SEQ ID NO: 221, SEQ ID NO: 222, or SEQ ID NO: 223 for LC-CDR 1; a nucleotide sequence or SEQ ID NO: 224, SEQ ID NO: 225, SEQ ID NO: 226, or SEQ ID NO: 227 for LC-CDR 2; and/or a nucleotide sequence of SEQ ID NO: 228, SEQ ID NO: 229, SEQ ID NO: 230, or SEQ ID NO: 231 for LC-CDR 3.

In any embodiment, a polynucleotide comprising a DNA sequence encoding any one of the antibodies described above is provided herein. For example, a polynucleotide may encode a variable heavy chain as described herein and may have a nucleotide sequence having at least about 80%, at least about 85% sequence identity, at least about 90% sequence identity, or at least about 95% sequence identity with a sequence of SEQ ID NO:117, SEQ ID NO:118, SEQ ID NO:119, SEQ ID NO:120 SEQ ID NO:121, SEQ ID NO:122, SEQ ID NO:123, SEQ ID NO:124, SEQ ID NO:125, SEQ ID NO:126, SEQ ID NO:127, SEQ ID NO:128, or SEQ ID NO:186. Additionally or alternatively, a polynucleotide encoding a variable heavy chain as described herein may have a nucleotide sequence of SEQ ID NO:117, SEQ ID NO:118, SEQ ID NO:119, SEQ ID NO:120 SEQ ID NO:121, SEQ ID NO:122, SEQ ID NO:123, SEQ ID NO:124, SEQ ID NO:125, SEQ ID NO:126, SEQ ID NO:127, SEQ ID NO:128, or SEQ ID NO:186.

A polynucleotide may further comprise a DNA sequence encoding a heavy chain leader sequence having at least about 80% sequence identity, at least about 85% sequence identity, at least about 90% sequence identity, or at least about 95% sequence identity with the sequence of SEQ ID NO:115 or SEQ ID NO:116. Additionally or alternatively, a nucleotide sequence for the DNA encoding a heavy chain leader sequence may be SEQ ID NO:115 or SEQ ID NO:116.

In further embodiments, a polynucleotide may further comprise a DNA sequence encoding a heavy chain constant region to form a DNA sequence encoding a full heavy chain, wherein the DNA sequence encoding the heavy chain constant region may have a nucleotide sequence having at least about 80% sequence identity, at least about 85% sequence identity, at least about 90% sequence identity, or at least about 95% sequence identity with the sequence of SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, or SEQ ID NO:187. Additionally or alternatively, a nucleotide sequence for the DNA encoding the heavy chain constant region may have nucleotide sequence of SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, or SEQ ID NO:187.

In any embodiment, a polynucleotide may have a DNA sequence encoding the full heavy chain of an antibody as described herein and may have a sequence having at least about 80% sequence identity, at least about 85% sequence identity, at least about 90% sequence identity, or at least about 95% sequence identity with the sequence of SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:188. Additionally or alternatively, a polynucleotide encoding the full heavy chain may have a nucleotide sequence of SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:188.

In any embodiment, a polynucleotide may encode the variable light chain and may have a sequence having at least about 80% sequence identity, at least about 85% sequence identity, at least about 90% sequence identity, or at least about 95% sequence identity with the sequence of SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, or SEQ ID NO:189. Additionally or alternatively, a polynucleotide encoding the variable light chain may have a nucleotide sequence of SEQ ID NO:154, SEQ ID NO:155, SEQ ID NO:156, SEQ ID NO:157, SEQ ID NO:158, SEQ ID NO:159, SEQ ID NO:160, SEQ ID NO:160, SEQ ID NO:162, SEQ ID NO:163, SEQ ID NO:164, SEQ ID NO:165, or SEQ ID NO:189.

A polynucleotide may further comprise a DNA sequence encoding a light chain leader sequence having a sequence having at least about 80% sequence identity, at least about 85% sequence identity, at least about 90% sequence identity, or at least about 95% sequence identity with the sequence SEQ ID NO:153. Additionally or alternatively, a nucleotide sequence for the DNA encoding the light chain leader has a sequence of SEQ ID NO:153.

A polynucleotide may further comprise a DNA sequence encoding a light chain constant region to form a DNA sequence encoding a full light chain, wherein the DNA sequence encoding the light chain constant region may have a sequence having at least about 80% sequence identity, at least about 85% sequence identity, at least about 90% sequence identity, or at least about 95% sequence identity with the nucleotide sequence of SEQ ID NO:166. Additionally or alternatively, a nucleotide sequence for the DNA encoding the light chain constant region may have a nucleotide sequence of SEQ ID NO:166.

In any embodiment, a polynucleotide has a DNA sequence encoding the full light chain of the antibody and may have a sequence having at least about 80% sequence identity, at least about 85% sequence identity, at least about 90% sequence identity, or at least about 95% sequence identity with the nucleotide sequence of SEQ ID NO:167, SEQ ID NO:168, SEQ ID NO:169, SEQ ID NO:170 SEQ ID NO:171, SEQ ID NO:172, SEQ ID NO:173, SEQ ID NO:174, SEQ ID NO:175, SEQ ID NO:176, SEQ ID NO:177, SEQ ID NO:178, or SEQ ID NO:190. Alternatively or additionally, a polynucleotide may have a DNA sequence encoding the full light chain of the antibody and may have a nucleotide sequence of SEQ ID NO:167, SEQ ID NO:168, SEQ ID NO:169, SEQ ID NO:170 SEQ ID NO:171, SEQ ID NO:172, SEQ ID NO:173, SEQ ID NO:174, SEQ ID NO:175, SEQ ID NO:176, SEQ ID NO:177, SEQ ID NO:178, or SEQ ID NO:190. In any embodiment for the production of a full antibody (e.g., an antibody comprising two pairs of a full heavy chain and a full light chain), polynucleotides encoding the full heavy chain and full light chain may be provided to a production cell (e.g., a hybridoma, bacteria, yeast, etc.) to produce the full antibody. The polynucleotides may be provided in any combination such that both a polynucleotide encoding a full heavy chain and a polynucleotide encoding a full light chain are provided.

Table 5 correlates the amino acid sequences described herein with the corresponding nucleotide sequences.

TABLE 5

| HEAVY CHAIN | | | LIGHT CHAIN | | |
|---|---|---|---|---|---|
| SEQUENCES | | | SEQUENCES | | |
| | Amino | Nucleotide | | Amino | Nucleotide |
| | Acid SEQ | SEQ ID | | Acid SEQ | SEQ ID |
| Region | ID NO | NO | Region | ID NO | NO |
| HC leader | 2 | 115 | LC leader | 33 | 153 |
| | 3 | 116 | LC | 91 | 154 |
| | | | variable | | |
| HC | 67 | 117 | | 92 | 155 |
| variable | | | | | |
| | 68 | 118 | | 93 | 156 |
| | 69 | 119 | | 94 | 157 |
| | 70 | 120 | | 95 | 158 |
| | 71 | 121 | | 96 | 159 |
| | 72 | 122 | | 97 | 160 |
| | 73 | 123 | | 98 | 161 |
| | 74 | 124 | | 99 | 162 |
| | 75 | 125 | | 100 | 163 |
| | 76 | 126 | | 101 | 164 |
| | 77 | 127 | | 102 | 165 |
| | 78 | 128 | | 184 | 189 |
| | 182 | 186 | LC | 66 | 166 |
| | | | constant | | |
| HC | 54 | 129 | LC | 103 | 167 |
| constant | | | full | | |
| | 55 | 130 | | 104 | 168 |
| | 56 | 131 | | 105 | 169 |
| | 57 | 132 | | 106 | 170 |
| | 58 | 133 | | 107 | 171 |
| | 59 | 134 | | 108 | 172 |
| | 60 | 135 | | 109 | 173 |
| | 61 | 136 | | 110 | 174 |
| | 62 | 137 | | 111 | 175 |
| | 63 | 138 | | 112 | 176 |
| | 64 | 139 | | 113 | 177 |
| | 65 | 140 | | 114 | 178 |
| | 134 | 187 | | 185 | 190 |
| HC full | 79 | 141 | | | |
| | 80 | 142 | | | |
| | 81 | 143 | | | |
| | 82 | 144 | | | |
| | 83 | 145 | | | |
| | 84 | 146 | | | |
| | 85 | 147 | | | |
| | 86 | 148 | | | |
| | 87 | 149 | | | |
| | 88 | 150 | | | |
| | 89 | 151 | | | |
| | 90 | 152 | | | |
| | 185 | 188 | | | |

### G. Kits

Kits for determining the presence of an ecstasy-class compound, such as MDA and/or MDMA in a sample, such as a biosample, are also provided herein.

In any embodiment, the kit may include, for example, in packaged combination, an antibody described above and a conjugate of an enzyme and an MDA analog and/or a conjugate of an enzyme and an MDMA analog. For example, the conjugate may correspond in structure to a Formula (I):

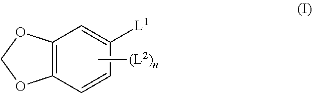

wherein
L¹ is

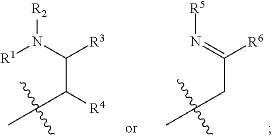

R¹ is hydrogen, an alkyl, or —CO(CF₃);
R² is hydrogen, an alkyl, or —(CH₂)_{b}—(X¹)—(Y¹)_{c};
   X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or

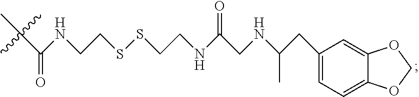 [SMILES: *C(=O)NCCSSCCNC(=O)CNC(C)Cc1ccc2c(c1)OCO2]

Y¹ is an enzyme;
b is 1 to 10;
c is zero or 1;
R³ is hydrogen, alkyl, or —(CH₂)_{d}—(X²)—(Y²)_{c};
   X² is —SH, —NH₂, —COOH, —CONH—, —S(CH₂)(CO)NH—, —CO(NH)(C₂H₄)S(CH₂)(CO)NH—, or

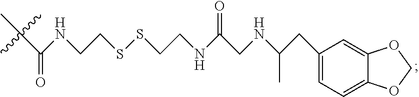 [SMILES: *C(=O)NCCSSCCNC(=O)CNC(C)Cc1ccc2c(c1)OCO2]

Y² is an enzyme;
d is 2 to 12;
e is zero or 1;
R⁴ is hydrogen or —(CH₂)_{f}(X³)—(Y³)_{g};
   X³ is —SH, —NH₂, —COOH, —CONH—, —S(CH₂)(CO)NH—, —CO(NH)(C₂H₄)S(CH₂)(CO)NH—, or

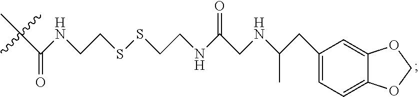 [SMILES: *C(=O)NCCSSCCNC(=O)CNC(C)Cc1ccc2c(c1)OCO2]

Y³ is an enzyme;
f is 1 to 12;
g is zero or 1;
a is zero or 1;
   R⁵ is hydrogen or an alkyl;
   R⁶ is hydrogen, an alkyl, or —(X⁴)—(Y⁴)ₕ;
      X⁴ is —SH, —NH₂, —COOH, —CONH—, —O(CH₂)(CO)(NH)(C₂H₄)(NH)(CO)(CH₂)Br, or —O(CH₂)(CO)(NH)(C₂H₄)(NH)(CO)(CH₂)S—;
   Y⁴ is an enzyme;
   h is zero or 1;
L² is —(CH₂)ᵢ—(X⁵)(Y⁵)ⱼ;
   X⁵ is —SH, —NH₂, —COOH, or —S(CH₂)(CO)NH—,
   Y⁵ is an enzyme;
   i is 1 to 12; and
   j is zero or 1.

Examples of a suitable enzyme, when present, include, but are not limited to, glucose-6-phosphate dehydrogenase (G6PDH), an alkaline phosphatase, or horseradish peroxidase, or a functional isoform and/or analog thereof. For example, the G6PDH conjugated to the conjugate may be a putative wild-type G6PDH or a mutant or recombinant form of G6PDH so long as the mutant or recombinant G6PDH can convert glucose-6-phosphate to 6-phospho-D-glucono-1,5-lactone. The functional isoform or analog of the alkaline phosphatase or horseradish peroxidase includes mutant or recombinant alkaline phosphatases or horseradish peroxidases that catalyze the same chemical reaction as wild-type or putative alkaline phosphatase (e.g., converting p-nitrophenyl phosphate (PNP) to p-nitrophenol) or horseradish peroxidase (e.g., oxidation of 3,3′,5,5′-tetramethylbenzidine (TMB)).

In an embodiment, the conjugate of (ii) is further defined wherein when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ is hydrogen; wherein when a is zero, R¹ is hydrogen or methyl, R² is —(CH₂)_{b}—(X¹)—(Y¹)_{c}, X¹ is —CONH—, c is 1, Y¹ is an immunogenic carrier or a label, R³ is methyl, R⁴ is hydrogen, then b is 5-10; wherein when a is zero, R² is —(CH₂)_{b}—(X¹)—(Y¹)_{c}, b is 3 or 4 X¹ is —COOH, c is zero, R³ is methyl, R⁴ is hydrogen, then R¹ is hydrogen; wherein when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b is 5-10; and/or wherein at least one of R¹, R², and R³ is neither hydrogen nor an alkyl.

In any embodiment, the kit may comprise reagents sufficient for a single use assay or for a multiple use assay. For example, where a single use of the assay is warranted, the kit may comprise an individually packaged volume or amount of the antibody sufficient for a single run of the assay and a volume or amount of the enzyme/MDA analog conjugate and/or enzyme MDMA analog conjugate sufficient to detect the presence of MDA and/or MDMA in the sample. Where multiples uses of the assay are warranted, the kit may comprise multiple, independently packaged volumes or amounts of the antibody and volumes or amounts of the enzyme/MDA analog conjugate and/or enzyme MDMA analog conjugate. Alternatively, the components of the kit may be packaged in larger volumes of the antibody and/or the conjugates where the artisan removes only a portion of said larger volume to run the assay to determine whether the sample contains MDA or MDMA. The components of the kit may otherwise be packaged at a relatively higher concentration of the antibody and/or the conjugates where the artisan removes only a portion of said concentrated volume and dilutes said volume prior to performing the assay to determine whether the sample contains MDA or MDMA. The antibody and/or conjugate may be provided in a powdered or lyophilized form for solubilization prior to use. The kit can further include a written description of a method in accordance with the present invention as described above.

### H. Methods and Assays

Compounds corresponding in structure to Formula (I), including where the compound comprises an enzyme, and antibodies raised against a compound corresponding in structure to Formula (I) may be employed as reagents in all types of immunoassays to determine the amount (e.g., concentration) of ecstasy-class compounds in samples having or suspected of having such compounds. The reagents may also be employed in multi-analyte immunoassays wherein the presence or absence of multiple analytes may be determined

Methods for determining the presence of an ecstasy-class compound (e.g., MDA and/or MDMA) in a sample, such as a biosample, are provided herein. In any embodiment, the method for determining the presence of MDA and/or MDMA in a sample includes providing to a medium: (i) the sample, (ii) a conjugate of an enzyme and an MDA analog, a conjugate of an enzyme and an MDMA analog, or a combination thereof, and (iii) an antibody described herein. The method further includes examining the medium for the presence of a complex comprising the antibody and MDA or MDMA (or both, if both the MDA conjugate and MDMA conjugate are utilized). In any embodiment of the method, the conjugate may correspond in structure to a compound of Formula (I):

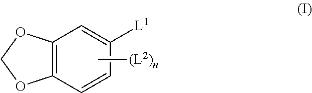

wherein
L¹ is

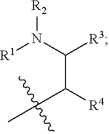

R¹ is hydrogen, an alkyl, or —CO(CF₃);
R² is hydrogen, an alkyl, or —(CH₂)_{b}—(X¹)—(Y¹)_{c};
   X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or

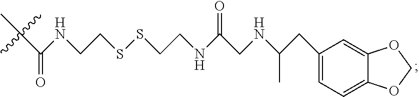 [SMILES: *C(=O)NCCSSCCNC(=O)CNC(C)Cc1ccc2c(c1)OCO2]

Y¹ is an enzyme;
b is 1 to 10;
c is zero or 1;
R³ is hydrogen, alkyl, or —(CH₂)_{d}—(X²)—(Y²)_{c};
   X² is —SH, —NH₂, —COOH, —CONH—, —S(CH₂)(CO)NH—, —CO(NH)(C₂H₄)S(CH₂)(CO)NH—, or

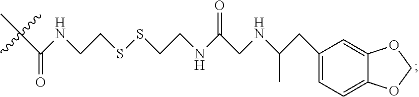 [SMILES: *C(=O)NCCSSCCNC(=O)CNC(C)Cc1ccc2c(c1)OCO2]

Y² is an enzyme;
d is 2 to 12;
e is zero or 1;
R⁴ is hydrogen or —(CH₂)_{f}(X³)—(Y³)_{g};
   X³ is —SH, —NH₂, —COOH, —CONH—, —S(CH₂)(CO)NH—, —CO(NH)(C₂H₄)S(CH₂)(CO)NH—, or

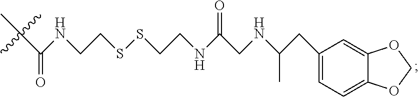 [SMILES: *C(=O)NCCSSCCNC(=O)CNC(C)Cc1ccc2c(c1)OCO2]

Y³ is an enzyme;
f is 1 to 12;
g is zero or 1;
a is zero or 1;
L² is —(CH₂)ᵢ—(X⁵)—(Y⁵)ⱼ;
   X⁵ is —SH, —NH₂, —COOH, or —S(CH₂)(CO)NH—,
   Y⁵ is an enzyme;
   i is 1 to 12; and
   j is zero or 1.

In any embodiment, the enzyme may include, but is not limited to, glucose-6-phosphate dehydrogenase (G6PDH), an alkaline phosphatase, or horseradish peroxidase.

In some embodiments, the compound of Formula (I) may further be defined by when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ is hydrogen; when a is zero, R¹ is hydrogen or methyl, R² is —(CH₂)_{b}—(X¹)—(Y¹) c X¹ is —CONH—, c is 1, Y¹ is an immunogenic carrier or a label, R³ is methyl, R⁴ is hydrogen, then b is 5-10; when a is zero, R² is —(CH₂)_{b}—(X¹)—(Y¹)_{c}, b is 3 or 4, X¹ is —COOH, c is zero, R³ is methyl, R⁴ is hydrogen, then R¹ is hydrogen; when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b is 5-10; and/or at least one of R¹, R², and R³ is neither hydrogen nor an alkyl.

Alternatively, the method may comprise a conjugate that may correspond in structure to a compound of Formula (I):

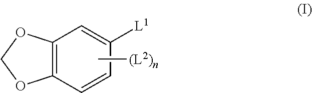

wherein
L¹ is

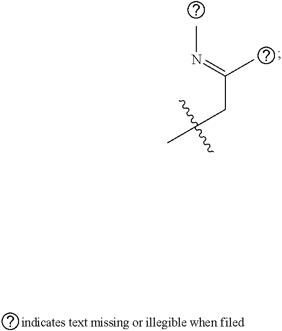 [SMILES: *C/C(C)=N/C]

a is zero or 1;
   R⁵ is hydrogen or an alkyl;
   R⁶ is hydrogen, an alkyl, or —(X⁴)—(Y⁴)ₕ;
      X⁴ is —SH, —NH₂, —COOH, —CONH—, —O(CH₂)(CO)(NH)(C₂H₄)(NH)(CO)(CH₂)Br, or —O(CH₂)(CO)(NH)(C₂H₄)(NH)(CO)(CH₂)S—;
   Y⁴ is an enzyme;
   h is zero or 1;
L² is —(CH₂)ᵢ—(X⁵)(Y⁵)ⱼ;
   X⁵ is —SH, —NH₂, —COOH, or —S(CH₂)(CO)NH—,
   Y⁵ is an enzyme;
   i is 1 to 12; and
   j is zero or 1.

In any embodiment, the enzyme may include, but is not limited to, glucose-6-phosphate dehydrogenase (G6PDH), an alkaline phosphatase, or horseradish peroxidase.

Regardless of which conjugate is used in the method, the method may further include: incubating the sample, conjugate, and antibody for a time sufficient for the antibody to bind to the ecstasy-class compound (e.g., MDA and/or MDMA) in the sample; adding a substrate for the enzyme to the sample; and measuring the activity of the enzyme. The substrate for the enzyme may be G6PDH substrate (e.g., D-glucose 6-phosphate), an alkaline phosphatase substrate (e.g., p-nitrophenol (PNP)), or a horseradish peroxidase substrate (e.g., 3,3′,5,5′-tetramethylbenzidine (TMB)). In such a method, the presence of a complex comprising the ecstasy-class compound (e.g., MDA and/or MDMA) and the antibody is proportional to the activity of the enzyme.

The sample tested in the method is not particularly limited. It may be organic or inorganic, biological (e.g., a “biosample”), non-biological, or environmental. Examples of a biological or biosample include, but are not limited to, urine, whole blood, plasma, serum, lymph, mucus, expressed breast milk, semen, stool, sputum, cerebral spinal fluid, tears, hair, saliva, cells, tissues, an organ, and/or a biopsy. In particular, the sample may be urine, blood, plasma, mucus, or saliva.

The assays described above may use various buffers to achieve and maintain a desired pH. The buffer is not particularly limited and may be borate, phosphate, carbonate, tris, barbital, and the like. Additional components, such as stabilizers for the medium, additional proteins (e.g., albumins to block non-specific and/or off-target antibody binding), organic solvents (e.g., formamide), quaternary ammonium salts, polyanions, surfactants, and binding enhances may be used as necessary. Incubation times and temperatures (e.g., for antibody binding) are not particularly limited and may be adjusted as necessary. Incubation temperatures may be about 5° C. to about 99° C., such as about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 99° C. Incubation times may be about 0.2 sec to about 6 h or overnight, for example about 5 min, about 10 min, about 15 min, about 30 min, about 45 min, about 1 h, about 1.5 h, about 2 h, about 3 h, about 4 h, about 5 h, or about 6 h. The specific time and temperature of the incubation may depend on the reagents used.

The concentration of ecstasy analyte that may be assayed generally varies from about 10⁻⁵ to about 10⁻⁹M, more usually from about 106 to about 10⁻⁸ M. Considerations, such as whether the assay is qualitative, semi-quantitative or quantitative (relative to the amount of analyte present in the sample), the particular detection technique and the concentration of the analyte will normally determine the concentrations of the various reagents.

Binding of the antibody for MDA and/or MDMA may result in the formation of an immune complex that can be detected directly or indirectly in numerous ways that are well known in the art. The immune complexes are detected directly, for example, when the antibodies employed are conjugated to a label. The immune complex is detected indirectly by examining for the effect of immune complex formation in an assay medium on a signal producing system.

Activation of the signal producing system depends on the nature of the signal producing system members. Activation methods include for example, light activation, addition of base of pH systems, radioactivity, and addition of substrate, wherein a cofactor may be also added if necessary.

In certain embodiments first and second labels may be employed and comprise a label pair. These label pairs may be, for example, a singlet oxygen generator or sensitizer and chemiluminescent reactant pair, an enzyme pair wherein a product of the first enzyme serves as a substrate for the second enzyme and a luminescent energy donor and acceptor pair, e.g., an energy donor or acceptor and a fluorescent compound. The signal will usually be initiated by and/or detected as electromagnetic radiation and will preferably be luminescence such as chemiluminescence, fluorescence, electroluminescence, or phosphorescence.

The examination for presence and level of the signal also includes the detection of the signal, which is generally merely a step in which the signal is read. The signal is normally read using an instrument, the nature of which depends on the nature of the signal. The instrument may be a spectrophotometer, fluorometer, absorption spectrometer, luminometer, chemiluminometer, actinometer, photographic instrument, and the like. The presence and level of signal detected is related to the presence and amount of the entactogen/analyte present in a sample above the predetermined cut-off level. Temperatures during measurements generally range from about 10° C. to about 70° C., more usually from about 20° C. to about 45° C., more usually about 20° C. to about 25° C. In one approach standard curves are formed using known concentrations of the analytes to be screened. Calibrators and other controls may also be used.

In any embodiment, the method may be or utilize an immunoassay, such as enzyme multiplied immunoassay technique (EMIT), an enzyme-linked immunosorbent assay (ELISA), a radioimmunoassay (RIA), an enzyme channeling immunoassay (ECIA), a fluorescence polarization immunoassay (FPIA), an enzyme modulate mediated immunoassay (EMMIA), a substrate labeled fluorescence immunoassay (SLFIA), a combined enzyme donor immunoassay (CEDIA), a particle enhanced turbidimetric inhibition immunoassay (PETINIA), a particle enhanced turbidimetric immunoassay (PETIA), a sol particle immunoassay (SPIA), a disperse dye immunoassay (DIA), a metalloimmunoassay (MIA), an enzyme membrane immunoassays (EMIA), and a luminoimmunoassays (LIA).

The EMIT assay is a homogenous enzyme immunoassay based on competition between a drug (e.g., MDA or MDMA) in the sample and the drug-conjugated to the enzyme (e.g., G6PDH). The method comprises the steps of: incubating the sample, drug-enzyme conjugate, and antibody for a time sufficient for the antibody to bind to the drug (e.g., MDA and/or MDMA) in the sample; adding an enzyme substrate to the sample; and measuring the activity of the enzyme, wherein the presence of the complex comprising the MDA and/or MDA and the antibody is proportional to the activity of the enzyme. The unbound enzyme conjugate converts the oxidized nicotinamide adenine dinucleotide (NAD⁺) to NADH and a change in absorbance at 340 nm is measured. Enzyme activity decreases upon binding to the antibody, which allows the analyte concentration in the sample to be measured in terms of enzyme activity. Enzyme activity can be measured as conventional in the art, such as measuring a change in fluorescence, radioactivity, or color of the sample. Measurement of the enzyme activity may be quantitative or qualitative. In light of the new SAMHSA guidelines, a new ecstasy EMIT assay may recover 80-96% MDA with low cross-reactivity for amphetamine and methamphetamine was developed.

### EXAMPLES

### General Synthesis Procedures

Materials and Equipment: The compounds were purified on a Shimadzu HPLC system (Riverwood, MD) equipped with a Silica-bond-C18 reverse phase column and Biotage LC (Charlotte, NC). The chemical reactions were monitored by TLC (thin layer chromatography) using Silica gel plates from Analtech Inc. (Newark, DE) and ESI-MS Waters HPLC (Milford, MA). The silica gel plates were visualized using UV short wave (254 nm). All chemicals were obtained from Sigma Aldrich (St. Louis, MO), Fluka (Waltham, MA), Thermo Scientific (Waltham, MA), VWR (Radnor, PA) and used as received. ¹H NMR was recorded on a Bruker UltraShield™ 600 MHz spectrometer (Bruker, Billerica, MA). Chemical shifts were reported in parts per million (ppm, δ) and related to tetramethylsilane or with deuterated solvent as internal reference. NMR abbreviations used are: s (singlet), brs (broad singlet), d (doublet), t (triplet), q (quartet), dd (doublet of doublets), qui (quintet) J (coupling constant), Hz (Hertz). ESI-MS spectra were recorded on a Water UPLC (Milford, MA) instrument at Siemens Healthineers RD department (Newark, DE). UV: Carry 60 was used for OD₂₈₀.

The following abbreviations have the meanings set forth below:
ACN—acetonitrile
AcOH—acetic acid
AcO⁻—acetate salt
Amph—amphetamine
mAmph—methamphetamine
BGG—bovine gamma globulin
Boc₂O—di-tert-butyl dicarbonate
BrAcSu—bromoacetic acid N-hydroxysuccinimide ester
BSA—bovine serum albumin
cBSA—cationized bovine serum albumin
BTG—bovine thyroglobulin
calcd.—calculated
CDCN-d₃—deuterated Acetonitrile having 3 deuterium atoms (for NMR spectra)
CDCl₃—deuterated Chloroform (for NMR spectra)
CFA—complete Freund's adjuvant
cm²—square centimeter
CV—column volume
DCM—dichloromethane
DI water—deionized water
DIPEA—N,N-diisopropylethylamine
DMF—N,N-dimethylformamide
DMSO—dimethyl sulfoxide
DNA—deoxyribonucleic acid
DTE—dithioerythritol
DTNB—5,5′-dithiobis(2-nitrobenzoic acid)
EDC or EDC·HCl—N-(3-dimethylaminopropyl)-N′-ethylcarbodiimide hydrochloride
eq.—molar equivalent
EDTA—ethylenediaminetetraacetic acid
EDTA-Na₂—ethylenediaminetetraacetic acid disodium salt
EMIT—enzyme multiplied immunoassay technique
ELISA—enzyme-linked immunosorbent assay
ESI-MS—electrospray ionization mass spectrometry
EtOAc—ethyl acetate
Fab fragment—antigen binding fragment
FBS—fetal bovine serum
g—grams
G6P—glucose-6-phosphate
G6PDH—glucose-6-phosphate native enzyme
G₆PDNa₂—glucose-6-phosphate disodium salt (substrate)
h—hour
HAT—hypoxanthine, aminopterin, and thymidine
HBS-N—10 mM HEPES, pH 7.4, 150 mM NaCl
HBS-P+—10 mM HEPES, pH 7.4, 150 mM NaCl, 0.02% surfactant P20
HPLC—high-performance liquid chromatography
HRP—horseradish peroxidase
HT—hypoxanthine and thymidine
IFM—incomplete Freund's adjuvant
IMDM—Iscove's Modified Dulbecco's Media
IP—intraperitoneal
K_{D}—equilibrium dissociation constant
KLH—keyhole limpet hemocyanin
KU—dimeric protein complex of nG₆PDH
LC—liquid chromatography
mAb—monoclonal antibody
min—minute
MDA—3,4-methylenedioxyamphetamine
MDA·HCl—3,4-methylenedioxyamphetamine hydrochloride salt
MDMA—3,4-methylenedioxymethamphetamine (also known as “ecstasy”)
MDMA·HCl—3,4-methylenedioxymethamphetamine hydrochloride salt
MeOH—methanol
MeOD-d₄—deuterated methanol having 4 deuterium atoms (for NMR spectra)
mg—milligram
μg—microgram
MHz—megahertz
mmol, mM—millimole
MsCl—mesyl chloride
MWCO—molecular weight cut-off
m/z—mass to charge ratio
NaOAc—sodium acetate
NaOAc buffer—sodium acetate buffer
β-NADH—nicotinamide adenine dinucleotide
NEt₃—triethylamine
nG6PDH—native glucose-6-phosphate dehydrogenase enzyme
nm—nanometer
NMR—nuclear magnetic resonance
OVA—ovalbumin isolated from chicken egg white (Sigma)
PB—phosphate buffer
PBS—phosphate buffered saline (20 mM sodium phosphate, 150 NaCl, pH 7.0)
Pd(dppf)₂Cl₂—[1,1′-bis(diphenylphosphino)ferrocene]dichloropalladium (II)
PEG—polyethylene glycol
PMK—piperonyl methyl ketone
R_{f}—retention factor in TLC analysis
RPM—rotations per minute
RU—response unit
rG6PDH—recombinant G6PDH enzyme
SDS-PAGE—sodium dodecyl sulphate-polyacrylamide gel electrophoresis
SuOH—N-hydroxysuccinimide
TCEP·HCl—tris(2-carboxyethyl) phosphine hydrochloride
THF—tetrahydrofuran
TFA—trifluoroacetic acid
TFAA—trifluoroacetic acid anhydride
TLC—thin-layer chromatography
TMB—3,3′,5,5′-tetramethylbenzidine
TRIS—tris(hydroxymethyl)aminomethane
TLC—thin layer chromatography
UV—ultraviolet
v/v—volume ratio

### Example 1: Synthesis and Activation of Compound 10

Synthesis of compound (2) (FIG. 1, Scheme 1): In an oven-dried microwave tube equipped with a magnetic stir bar compound (1) (500 mg, 2.18 mmol), Pd(dppf)₂Cl₂ catalyst (80 mg, 5 mol %), and LiCl (~90 mg) are loaded and the tube was capped. THF (6 ml) was then added, followed by ethoxy-4-oxobutylzinc bromide-THF solution (1.6 eq., 3.49 mmol, 7 mL). The resulting reaction mixture was heated to 60° C. for 8 h. The reaction was quenched with Si-Thiol resin (~50 mg) for 15 min, the precipitate was filtered off. The solvent was removed on a rotatory evaporator to give a pale-yellow oil which was purified on a Biotage LC (Hexanes/Ethyl Acetate) system equipped with an Ultra-SNAP 10 g column. Fractions containing the useful product were pooled out, concentrated on a rotatory evaporator and further dried on an oil pump to give 240 mg (0.909 mmol) of compound (2) as a pale-yellow oil in 46% yield. ESI-MS m/z calcd, for [C₁₄H₁₆O₅Na]⁺ 287.09, found 287.23 (ret. Time 4.47 min). ¹H NMR (600 MHz, CDCl₃) 10.14 (s, 1H), 7.30 (s, 1H), 6.71 (s, 1H), 6.03 (s, 2H), 4.13 (q, J=7.14 Hz, 2H), 3.01-2.99 (m, 2H), 2.37 (t, J=7.26 Hz, 2H), 1.96-1.91 (m, 2H), 1.26 (t, J=7.09 Hz, 3H). ¹³C NMR (150 MHz, CDCl₃) 189.55, 173.26, 152.58, 147.06, 142.12, 128.53, 110.69, 109.16, 102.09, 60.62, 33.71, 31.41, 27.81, 14.43.

Synthesis of compound (3) (FIG. 1, Scheme 1): Compound (2) (240 mg, 0.909 mmol) was suspended in a mixture of formic acid:ethanolamine (1:1 molar ratio, 2 mL), then, nitroethane (110 μL, 1.5 eq.) was added. The resulting reaction mixture was heated to 60° C. for 4 h. As the reaction proceeds, it turns bright yellow. After completion, the reaction mixture was diluted with ice-water (10 mL) and extracted with EtOAc (3×20 mL). The organic solvent was removed on a rotatory evaporator to give a bright yellow oil and purified on a Biotage LC (Hexanes/Ethyl Acetate). Fractions containing the useful product were pooled out, concentrated on a rotatory evaporator and further dried on an oil pump to give 220 mg (0.68 mmol) of compound (3) as a bright yellow oil in 75% yield. ESI-MS m/z calcd, for [C₁₆H₂₀NO₆]⁺ 322.13, found 322.24. ¹H NMR (600 MHz, CDCl₃) 8.14 (s, 1H), 6.76 (s, 1H), 6.72 (s, 1H), 6.00 (s, 2H), 4.13 (q, J=7.08 Hz, 2H), 2.62 (dd, J=9.06, 7.62 Hz, 2H), 2.34 (s, 3H), 2.30 (t, J=7.32 Hz, 2H), 1.86-1.81 (m, 2H), 1.25 (t, J=7.14 Hz, 3H). ¹³C NMR (150 MHz, CDCl₃) 173.20, 149.29, 147.92, 146.36, 137.02, 132.44, 124.20, 110.25, 109.07, 101.76, 60.66, 33.63, 32.96, 26.48, 14.43, 14.18.

Synthesis of Compound (5) (FIG. 1, Scheme 1): Compound (3) (95 mg, 0.30 mmol) was dissolved in anhydrous THF (2 mL), the reaction mixture was blanked with Argon, cooled on an ice bath; then, LiAlH₄-THE solution (7 eq., 2.10 mL THF solution 1M) was added dropwise through a septum. The resulting reaction mixture was then heated to 60-75° C. for 16 h under Argon positive pressure. The reaction mixture was cooled on an ice bath, then quenched by slow addition of NaOH solution (80 μL 3.75 N) followed by water (240 μL). The formed precipitate was filtered-off and washed with THF (5×10 mL). The filtrate was concentrated on a rotatory evaporator to give the reduced intermediate solution {ESI-MS m/z for [C₁₄H₂₂NO₃]⁺ 252.16, found 252.25, ret. time 4.52 min}. This solution was concentrated to ~3 mL, then NEt₃ (100 μL) was added followed by BoczO (180 μL, 0.36 mmol, as 2M THF solution). The resulting reaction mixture was stirred at rt for 4 h and monitored by TLC (EtOAc:Hexane 1/1 v/v, R_{f_prod}=0.50). The product was purified by preparative TLC, the third spot form bottom (R_{f}~0.50) was collected. An amount of 65 mg (0.19 mmol) of compound (5) was recovered (62% yield as a colorless oil). ESI-MS m/z for [C₁₉H₂₉NO₅Na]⁺ 374.19, found 374.35. ¹H NMR (600 MHz, CDCl₃) 6.65 (s, 1H), 6.60 (s, 1H), 5.90-5.87 (m, 2H), 4.50-4.49 (m, 1H), 3.80-3.78 (m, 1H), 3.69-3.67 (m, 2H), 2.92 (m, 1H), 2.66-2.56 (m, 3H), 2.40 (brs, 1H), 1.64 (brs, 4H), 1.42 (s, 9H), 1.25 (brs, 1H), 1.11 (d, J=7.0 Hz, 3H).

Synthesis of compound (6) (FIG. 1, Scheme 1): Compound (5) (65 mg, 0.19 mmol) was suspended in DCM (0.4 mL), then NEt₃ (59 μL, 2.3 eq.) was added. The resulting reaction mixture was cooled on an ice-bath (0-4° C.), then, mesyl chloride (MsCl) (17 μL, 0.221 mmol, 1.2 eq.) was added. The resulting reaction mixture was allowed to warm up to rt and completes after 1 h. The solvent was removed on a rotatory evaporator to give a colorless oil. ESI-MS m/z calcd, for [C₂₀H₃₁NO₇Sna]⁺ 452.17, found 452.39.

Synthesis of compound (7) (FIG. 1, Scheme 1): Compound (6) was dissolved in THF (0.2 mL); then, a solution of potassium thioacetate (25.04 mg, 1.2 eq., in 0.2 mL DMF) was added. The resulting reaction mixture was heated to 80° C. and it completed after 2 h. The volatiles were removed on a rotatory evaporator to give 110 mg of crude product as an orange oil. The crude product was suspended in water (5 mL) and extracted with EtOAc (3×10 mL). The combined organic layers dried over Na₂SO₄, concentrated on a rotatory evaporator and further dried on an oil pump to give 42 mg (0.102 mmol) of compound (7) in 55% yield. ESI-MS m/z for [C₂₁H₃₀NO₅Sna]⁺ 432.18, found 432.37. ¹H NMR (600 MHz, CDCl₃) 6.63 (s, 1H), 6.62 (s, 1H), 5.88 (dd, J=5.41, 1.51 Hz, 2H), 4.40 (s, 1H), 3.79 (s, 1H), 2.91 (t, J=7.02 Hz, 2H), 2.79-2.70 (m, 1H), 2.62-2.50 (m, 3H), 2.32 (s, 3H), 1.65-1.58 (m, 4H), 1.42 (s, 9H), 1.09 (d, J=6.66 Hz, 3H).

Synthesis of Compound 8 and E5 hapten (9) (FIG. 1, Scheme 1): Compound (7) (42 mg, 0.102 mmol) was suspended in MeOH (1 mL), then NaOH (4 eq. 10 N) was added, the hydrolysis of the acetyl group and subsequent dimerization of the by-product took place to generate compound (8). ESI-MS m/z calcd. for [C₃₈H₅₆N₂O₈S₂Na]⁺ 755.34, found 755.64. The solvent was evaporated on a rotatory evaporator to give a pale-yellow oil which was suspended in DCM (20 mL) and washed with DI water (2×10 mL). The volatiles were removed on a rotatory evaporator, then MeOH (1 mL) and TFA (150 μL) were added and the resulting reaction mixture was stirred at 60° C. for 3 days. The volatiles were removed in vacuo to give 40 mg of E5 hapten (9) in quantitative yield. ESI MS m/z calcd, for [C₂₈H₄₁N₂O₄S₂]⁺ 533.25, found 533.48 (ret. Time 2.53 mins). ¹H NMR (600 MHz, MeOD-d4) 6.72 (s, 2H), 6.71 (s, 2H), 5.90 (s, 4H), 3.44-3.42 (m, 2H), 2.94 (dd, J=13.98, 6.18 Hz, 2H), 2.76 (dd, J=13.92, 8.82 Hz, 2H), 2.70 (t, J=7.14 Hz, 4H), 2.59 (t, J=8.06 Hz, 4H), 1.77-1.72 (m, 4H), 1.67-1.62 (m, 4H), 1.25 (d, J=6.60 Hz, 6H).

Activation of E5 hapten (9) (FIG. 1, Scheme 1): E5 hapten (9) (28 mg, 0.05 mmol) was suspended in MeOH (1 mL), then, NaOAc buffer (0.4 mL, 25 mM, pH 4.5) was added. The resulting mixture was not homogenous. TCEP·HCl (12 mg, 0.9 eq.) was added. The resulting reaction mixture was stirred at rt and completed after 1 h (reaction becomes clear when completed) to give activated E5 hapten (10). ESI-MS m/z for [C₁₄H₂₂NO₂S]⁺ 268.14, found 268.24 (ret. time 4.87 min). The concentration of the E5-hapten (9) solution was 20 mg/mL.

### Example 2: Preparation of Protein Conjugates and Immunogens with Activated E5 Hapten (10)

Preparation of the protein conjugate and immunogens (FIG. 2, Scheme 2): Each protein (OVA, cBSA, KLH) was dissolved in PB (100 mM, pH 7.4) to make a 5 mg/mL solution. BrAcSu (21 mg, 0.089 mmol) was dissolved in DMF (1.05 mL) to make a 20 mg/mL solution. The BrAcSu-DMF solution was added dropwise to the protein solutions (Table 6, Column 3) in a cold room (2-8° C.). The resulting reactions were stirred for 3 h in a cold room. The BrAcOVA activated protein was purified on a G25M Sephadex® column (CV=71 mL; diameter: 1.5 cm, height 40 cm), whereas the BrAc-cBSA and BrAc-KLH were purified by Amicon stirring cells (10000 MWCO, 100 mM PB, pH 7.4, 5×20 mL). Each protein solution volume was adjusted to 5 mL. The activated proteins were cooled on an ice bath, then, a solution of activated E5 hapten (10) was added dropwise (Table 6, Column 4).

The resulting reaction mixtures are stirred at rt for 3 h. The E5-OVA (44) conjugate was purified on a G25M Sephadex® column (CV=71 mL; diameter: 1.5 cm, height 40 cm,) whereas E5-cBSA1 (45) conjugate, E5-cBSA2 (46) conjugate, and E5-KLH (47) immunogen were purified by dialysis using PB (100 mM, pH 7.0) on Amicon stirring cell (10000 MWCO). The amounts recovered are presented in Column 5.

TABLE 6

| | Protein \| | Molx \| m_{BrAcOSu} \| | MolXₕₐₚₜₑₙ \|mₕₐₚₜₑₙ \| | Amounts |
|---|---|---|---|---|
| Conj. ID | mass | v_{DMFsol} | Vₛₒₗᵤₜᵢₒₙ | recovered |
| 1 | 2 | 3 | 4 | 5 |
| E5-OVA (44) | OVA \| 15 mg | 40x \| 3.34 mg \| | 40x \| 3.77 mg \| 190 | 0.50 mg/mL \| |
| | | 165 μL | μL | 11.50 mL |
| E5-cBSA1 (45) | cBSA1 \| 20 | 60x \| 4.22 mg \| | 60x \| 4.80 mg \| 240 | 2.01 mg/mL \| |
| | mg | 211 μL | μL | 6.6 mL |
| E5-KLH (47) | KLH \| 20 mg | 800x \| 5.94 mg \| | 800x \| 6.76 mg \| 340 | 0.84 mg/mL \| |
| | | 300 μL | μL | 11.0 mL |
| E5-cBSA2 (46) | cBSA2 \| 20 | 30x \| 2.11 mg \| | 30x \| 2.40 mg \| 120 | 1.43 mg/mL \| |
| | mg | 106 μL | μL | 9.0 mL |

### Example 3: Synthesis and Activation of E1 Hapten (51)

Synthesis of E1 hapten (51) (FIG. 3, Scheme 3): Route for intermediate “a” is selected as an example: MDA·HCl (50 mg, 0.232 mmol) was suspended in DMF (0.500 mL), K₂CO₃ (70 mg, 0.506 mmol) and LiBr (~80 mg) were added and the resulting reaction mixture was stirred at rt for 5 min. In a separate vial, a solution of linker 50a (46 mg, 117 mmol in 1.00 mL THF) was prepared; 200 μL of this solution was added every 2 h and the reaction progression was monitored by LC-MS. The reaction was completed after 48 h. The solvent was removed in vacuo to give a pale-yellow oil which was dissolved in Water/ACN (2/1 v/v 2 mL total volume containing 0.1% AcOH) and injected into a Shimadzu HPLC system. Fractions containing the useful product are pooled out, concentrated on a rotatory evaporator and lyophilized overnight to give 34 mg (0.055 mmol) of an E1 hapten (51a) as white powder in 50% yield. ESI-MS m/z for [C₂₈H₃₉N₄O₆S₂]⁺ 592.23, found 591.42. ¹H NMR (600 MHz, MeOD-d₄) 6.77 (d, J=7.87 Hz, 1H), 6.75 (d, J=1.53 Hz, 1H), 6.70 (dd, J=7.88, 1.53 Hz, 1H), 5.93 (s, 2H), 3.51-3.47 (m, 3H), 3.42 (d, J=16.23 Hz, 1H), 3.03 (q, J=6.66 Hz, 1H), 2.82-2.78 (m, 3H), 2.61 (dd, J=13.48, 7.44 Hz, 1H), 1.97 (s, 3H), 1.13-1.12 (m, 3H). ¹³C NMR (125 MHz, MeOD-d₄) 177.68, 172.04, 149.41, 147.94, 133.36, 123.61, 110.68, 109.35, 102.43, 56.78, 42.87, 39.45, 38.52, 22.51, 18.85. Routes a and b used different linkers but they generated the same activated hapten by following the same coupling chemistry and route a is selected due to linker availability.

Activation of E1 hapten (Scheme 3) (route b): E1 hapten (51b) (14.89 mg, 0.043 mmol) was suspended in NaOAc buffer (25 mM, pH 4.2, 0.371 mL). The solution was degassed with Argon for 1 min, then, TCEP·HCl (0.8 eq., 10 mg) was added, the resulting reaction mixture was stirred at rt for 1 h. The vial was then connected to a vacuum line and evaporated to dryness to remove MeSH by-product. The vial was dried for additional 2 h on an oil pump. The formation of the activated E1 hapten (52) was confirmed by TLC (DCM/MeOH 8/2, R_{f-SM}=0.80, R_{f-Prod}=0.20) and ESI-MS m/z for [C₁₄H₂₁N₂O₃S]⁺ 297.1, expected 297.1. After evaporation, the activated E1 hapten (52) was dissolved in PB (0.1M pH 8.0, 1.08 mL) and the solution was cooled on an ice bath. This solution was used to prepare the E1-OVA conjugate (55) and E1-KLH immunogen (56). (FIG. 3, Scheme 3)

### Example 4: Preparation of Protein Conjugates and Immunogens with Activated E1 Hapten (52)

Synthesis of E1-OVA conjugate (55) (FIG. 3, Scheme 3): Ovalbumin (10 mg) was suspended in PB (100 mM, pH 8.0, 3.0 mL). The protein solution was cooled on an ice bath, then a solution of BrAcSu-DMF (2.21 mg, 0.111 mL, 20 mg/mL 40 mol excess) was added dropwise. The resulting reaction mixture was rocked in a cold room (2-8° C.) for 4 h, then buffer exchanged on an Amicon stirring cell (9000 MWCO). 2 mL of BrAc-OVA activated protein solution was recovered. The activated protein solution was cooled on an ice bath, then the activated E1 hapten (52) formed via route b (3.80 mg, 0.276 mL, 40 mol excess 13.79 mg/mL) was added dropwise. The resulting reaction mixture was allowed to warm up to rt for 15 min, then, it was transferred to a cold room (2-8° C.) and stirred for 2.5 h. The E1-OVA conjugate (55) was purified by Amicon stirring cell (9000 MWCO, 6×15 mL) using PB (100 mM, pH 7.0) and filtered through Corning filters 0.20 μm. 6 mg of compound (55) was recovered.

Synthesis of E1-KLH immunogen (56) (FIG. 3, Scheme 3): Protein KLH (20 mg) was suspended in PB (100 mM, pH 8.0, 4.0 mL). The enzyme solution was cooled on an ice bath, then a solution of BrAcSu-DMF (3.50 mg, 0.175 mL, 20.0 mg/mL) was added dropwise. The resulting reaction mixture was rocked in a cold room (2-8° C.) for 16 h, then buffer exchanged on an Amicon stirring cell (9000 MWCO). The next day, the protein was purified by Amicon stirring cell (30000 MWCO, 8×15 mL of PB 100 mM, pH 8.0). After buffer exchange, 4 mL of BrAc-KLH activated protein solution was recovered. The protein solution was transferred onto a plastic tube, cooled on an ice bath, then the activated E1 hapten (52) formed via route b (7.17 mg, 0.520 mL, 13.79 mg/mL) was added dropwise. The resulting reaction mixture was rocked in a cold room (2-8° C.) for 16 h. The E1-KLH immunogen (56) was purified by Amicon stirring cell (30000 MWCO) using PB (100 mM, pH 7.0, 6×15 mL). 16 mg of E1-KLH immunogen (56) was recovered.

### Example 5: Synthesis and Activation of E2-Hapten (60)

Synthesis of Compound 11 (FIG. 4, Scheme 4): MDA·HCl (50 mg, 0.232 mmol) was dissolved in acetonitrile (1 mL), then aqueous K₂CO₃ (38 mg, 0.27 mmol in 100 μL water) was added. The formed precipitate was filtered through a 20 μm filter, the solid was further extracted with DMF (2×0.5 mL). The combined clear solutions were collected into a 50 mL round bottom flask. Tert-Butyl-5-bromovalerate (1.2 eq. 52 μL, 0.27 mmol) and LiBr (50 mg) were added was added and the resulting reaction mixture was stirred at rt for 48 h. After this time, more linker (0.2 eq., 8.7 μL) and K₂CO₃ (6 mg, 0.2 eq) are added and the reaction mixture was heated to 40° C. for an additional 6 h. The product was purified by Biotage LC (Hexanes/Ethyl Acetate). Fractions containing the product were pooled out, concentrated on a rotatory evaporator and further dried on an oil pump to give 53 mg (0.158 mmol) of compound (11) as a pale-yellow oil in 68% yield. ESI-MS m/z for [C₁₉H₃₀NO₄]⁺ 336.22, found 336.41 (mixture of isomers). ¹H NMR (600 MHz, CDCl₃) 6.72 (d, J=7.88 Hz, 1H), 6.67 (d, J=1.4 Hz, 1H), 6.64 (dd, J=7.88, 1.4 Hz, 1H), 6.21 (s, 2H), 5.92 (s, 2H), 3.03-3.01 (m, 1H), 2.96-2.92 (m, 1H), 2.82-2.78 (m, 1H), 2.72-2.67 (m, 1H), 2.59 (dd, J=13.37, 8.24 Hz, 1H), 2.20 (t, J=7.23 Hz, 2H), 1.64-1.58 (m, 4H), 1.43 (s, 9H), 1.15 (d, J=6.37 Hz, 3H). ¹³C NMR (150 MHz, CDCl₃) 176.99, 172.65, 147.78, 146.25, 131.77, 122.26, 109.49, 108.29, 100.89, 80.23, 54.90, 45.50, 41.27, 35.02, 28.09, 27.64, 23.56, 22.53, 17.83.

Synthesis of compound 12 (FIG. 4, Scheme 4): Compound 11 (53 mg, 0.158 mmol) was suspended in anhydrous dichloromethane (2 mL) under Argon. DIPEA (82 μL, 0.473 mmol, 3 eq.) was added and the resulting reaction mixture was cooled on an ice-bath. Using an oven dried glass syringe, trifluoracetic anhydride (66 μL, 99.36 mg, 3 eq.) was then added. The resulting reaction mixture was allowed to warm up to rt and stirred for 2 h [TLC analysis (EtOAc/Hexane 2/1 v/v showed the product formation at R_{f-Prod}=0.7, R_{fsM}=0.2]. The volatiles were removed on a rotatory evaporator to give a yellow oil which was purified by Biotage LC (Solvent A: Hexanes/EtOAc 3/1 and Solvent B: DCM/MeOH 9/1, v/v). Fractions containing the useful product were pooled out, concentrated on a rotatory evaporator and further dried on an oil pump to give 35 mg (0.081 mmol) of compound (12) in 51% yield as a yellow oil. ESI-MS m/z calcd, for [C₂₁H₂₉F₃NO₅Na]⁺ 454.20, found 454.40 (mixture of rotamers). ¹H NMR (600 MHz, CDCl₃) 6.74 (d, J=7.89 Hz, 0.6H), 6.72 (d, J=8.0 Hz, 0.4H), 6.66 (d, J=1.59 Hz, 0.5H), 6.61 (m, 1H), 6.60-6.58 (m, 1H), 5.94-5.93 (m, 2H), 4.15-4.12 (m, 0.5H), 3.77-3.74 (m, 0.5H), 3.43-3.38 (m, 0.5H), 3.23-3.16 (m, 1.5H), 2.97-2.93 (m, 0.5H), 2.83 (dd, J=13.36, 5.33 Hz, 0.5H), 2.74 (dd, J=13.62, 6.99 Hz, 0.5H), 2.64 (dd, J=13.37, 9.25 Hz, 0.5H), 2.28-2.26 (m, 1H), 2.20-2.18 (m, 1H), 1.76-1.73 (m, 0.5H), 1.67-1.62 (m, 2H), 1.51-1.50 (m, 2H), 1.49-1.48 (m, 9H), 1.38 (d, J=6.86 Hz, 1.5H), 1.19 (d, J=6.60 Hz, 1.5H). 19F NMR (564 MHz, CDCl₃) −68.46, −69.61. ¹³C NMR (150 MHz, CDCl₃) 172.63, 172.26, 156.92-156.10 (m, C—F coupling), 147.88, 147.74, 146.55, 146.26, 132.21, 130.81, 122.12, 122.01, 116.64 (q, J=287.6 Hz), 116.34 (q, J=288.53 Hz), 109.32, 109.24, 108.43, 108.27, 101.01, 100.89, 80.43, 80.28, 59.27, 54.88, 54.86, 47.94, 42.77, 41.47, 39.11, 35.00, 34.80, 29.68, 29.01, 28.10, 28.06, 27.65, 22.78, 22.11, 18.24, 17.61.

Synthesis of E2 hapten (60) (FIG. 4, Scheme 4): Compound 12 (35 mg, 0.081 mmol) was suspended in DCM (0.4 mL) then trifluoroacetic acid (124 μL, 185 mg, 20 eq.) was added. The resulting reaction mixture was stirred overnight and monitored by TLC (DCM/MeOH 9/1 v/v R_{f_prod}=0.30). The solvent was removed on a rotatory evaporator to give a yellow oil was then purified on a Biotage LC (DCM/MeOH). The product eluted after 5 CV, fractions containing the product were collected and concentrated in vacuum to give 30 mg (0.080 mmol) of E2 hapten (60) as a yellow oil in 99% yield. ESI-MS m/z calcd, for [C₁₇H₁₉F₃NO₅]⁻ 374.12, found 374.32. ¹H NMR (600 MHz, CDCl₃) 6.74 (d, J=7.88 Hz, 0.6H), 6.72 (d, J=7.93 Hz, 0.4H), 6.65 (d, J=1.36 Hz, 0.4H), 6.61 (m, 0.6H), 6.60-6.55 (m, 1H), 5.94 (s, 0.6H), 5.92 (s, 0.4H), 4.16-4.12 (m, 0.6H), 3.74-3.71 (m, 0.4H), 3.44-3.39 (m, 0.6H), 3.25-3.17 (m, 1.4H), 3.00-2.94 (m, 0.5H), 2.83 (dd, J=13.38, 5.31 Hz, 0.6H), 2.74 (dd, J=13.64, 6.81 Hz, 0.5H), 2.64 (dd, J=13.34, 9.27 Hz, 0.6H), 2.42 (t, J=7.02 Hz, 1H), 2.33 (t, J=6.65 Hz, 1H), 1.78-1.62 (m, 2.6H), 1.56-1.50 (m, 2H), 1.39 (d, J=6.85 Hz, 1.33H), 1.19 (d, J=6.58 Hz, 1.66H). 19F NMR (564 MHz, CDCl₃) −68.45, −69.58.

Activation of E2 hapten (60) (FIG. 4, Scheme 4): The E2-hapten (60) (14 mg, 0.037 mmol) was suspended in DMF (0.280 mL), SuOH (6 mg, 0.044 mmol) was added followed by EDC·HCl (8 mg, 0.0407 mmol). The resulting reaction mixture was stirred at rt for 48 h to give activated E2 hapten (61). ESI-MS m/z calcd. for [C₂₁H₂₃F₃N₂NaO₇]⁺ 495.20, found 495.40.

### Example 6: Preparation of Protein Conjugates and Immunogens with Activated E2-Hapten (61)

Synthesis of E2-OVA conjugate (65) (FIG. 4, Scheme 4): Ovalbumin (20 mg) was suspended in PB (50 mM, pH 7.5, 4 mL) in a vial equipped with a magnetic stir bar. The protein solution was cooled on an ice-bath, then activated E2-hapten (61)-DMF solution (70 μL, 3.51 mg, 20×) was added dropwise. The resulting reaction mixture was stirred at rt overnight (16 h). The reaction mixture was then dialyzed with PB (1 L of 50 mM, pH 12.8) for 7 days in a cold room to deprotect the trifluoroacetate group, then, with PB (3×1 L 50 mM pH 7.0). The conjugate was filtered off through Corning filters (20 μm). After filtration, 5.40 mL of E2-OVA (65) conjugate with a concentration of 3.70 mg/mL was recovered.

Synthesis of E2-KLH immunogen (66) (662×) (FIG. 4, Scheme 4): KLH (20 mg) was suspended in PB (50 mM, pH 7.5, 4 mL) in a vial equipped with a magnetic stir bar. The protein solution was cooled on an ice-bath, then, activated E2 hapten (61)-DMF solution (156 μL, 7.80 mg) was added dropwise. The resulting reaction mixture was stirred at rt overnight (16 h). The reaction mixture was then dialyzed against PB (1 L of 50 mM, pH 12.8) for 7 days in a cold room (2-8° C.) to deprotect the trifluoroacetate group, then with PB (3×1 L of 50 mM, pH 7.0). After dialysis, 6.5 mL of E2-KLH (66) (1000×) conjugate with a concentration of ~3.00 mg/mL was recovered.

Synthesis of E2-KLH (67) (220×) (FIG. 4, Scheme 4): KLH (20 mg) was suspended in PB (50 mM, pH 7.5, 4 mL) in a vial equipped with a magnetic stir bar. The protein solution was cooled on an ice-bath, then, activated E2 hapten (61)-DMF solution (50 μL, 2.6 mg) was added dropwise. The resulting reaction mixture was stirred at rt overnight (16 h). When the hapten-DMF solution was added, the reaction became milky, and it became clear as reaction proceeds. The reaction mixture was then dialyzed against PB (1 L of 50 mM, pH 12.8) for 7 days in a cold room to deprotect the trifluoroacetate group, then against PB (3×1 L of 50 mM, pH 7.0). After dialysis, 6.5 mL of E2-KLH (67) (220×) conjugate with a concentration of ~3.0 mg/mL was recovered.

### Example 7: Synthesis and Activation of E3 Hapten (70)

Synthesis of compound (14) (FIG. 5, Scheme 5): MDMA·HCl (40 mg, 0.174 mmol) was suspended in DMF (0.500 mL), K₂CO₃ (96.40 mg, 0.70 mmol) was added and the resulting reaction mixture was stirred at rt for 5 min, then LiBr (~30 mg) was added followed by ethyl-5-bromovalerate linker 13 (108 mg, 3 eq.) the resulting reaction mixture was heated to 50-70° C. for 24 h. The solvent was removed on a rotatory evaporator to give a white suspension which was extracted with in ACN/MeOH 8/2 v/v (2×4 mL). The product was purified by Biotage LC (Solvent A: Hexanes/EtOAc 3/1; Solvent B: dichloromethane/MeOH 9/1). Fractions containing the useful product (as a mixture of two rotamers) were pooled out, concentrated in vacuo to give 47 mg (0.146 mmol) of compound 14 as a pale-yellow oil in 84% yield. ESI-MS m/z calcd. for [C₁₈H₂₈NO₄]⁺ 322.19, found 322.34. ¹H NMR (600 MHz, CDCl₃) 6.69 (d, J=7.80 Hz, 1H), 6.64 (d, J=1.80 Hz, 1H), 6.58 (dd, J=8.0, 1.7 Hz, 1H), 5.88 (s, 2H), 4.10 (q, J=7.2 Hz, 2H), 2.86-2.84 (m, 2H), 2.46-2.43 (m, 2H), 2.32-2.26 (m, 6H), 1.63-1.58 (m, 2H), 1.53-1.48 (m, 2H), 1.24-1.22 (m, 3H), 0.91 (d, J=6.0 Hz, 3H).

Synthesis of the E3 hapten (70) (FIG. 5, Scheme 5): Compound (14) (47 mg, 0.146 mmol) was dissolved in MeOH (2 mL), then NaOH (10 N, 250 μL) was added. The resulting reaction mixture was stirred at rt for 4 h then, HCl (10N, 250 μL) was added until pH was ~4. The solvent was removed in vacuo to give a colorless powder which was extracted with ACN (2×8 mL). The compound was purified by Shimadzu HPLC to give E3-hapten (70) (38 mg, 0.108 mmol) in 74% yield (as acetate salt). The acetate salt was dissolved in MeOH/Water (50/50 v/v 1 mL) and treated with TFA (~40 μL) then evaporated to dryness to get the TFA salt of E3 hapten (70) (42 mg). ESI-MS m/z calcd, for [C₁₆H₂₄NO₄]⁺ 294.17, found 294.29. ¹H NMR (600 MHz, CDCl₃) 6.82 (d, J=1.38 Hz, 1H), 6.78 (d, J=7.89 Hz, 1H), 6.74 (dd, J=7.88, 1.38 Hz, 1H), 5.92 (s 1H), 3.62-3.58 (m, 1H), 3.16-3.12 (m, 3H), 2.80 (s, 3H), 2.69 (dd, J=12.99, 10.83 Hz, 1H), 2.31 (t, J=6.81 Hz, 2H), 1.96 (s, 3H from AcO salt.), 1.81-1.77 (m, 2H), 1.71-1.67 (m, 2H), 1.20 (d, J=6.65 Hz, 3H).

Activation of E3 hapten (70) (FIG. 5, Scheme 5): E3 hapten (70) (42 mg, 0.108 mmol) was dissolved in DMF (2.1 mL), then SuOH (18.98 mg, 0.229 mmol) was added followed by EDC·HCl (27.65 mg, 0.145 mmol). The resulting reaction mixture was stirred at rt for 48 h to give activated E3 hapten (71). ESI-MS m/z calcd. for [C₂₀H₂₇N₂O₆]′ 391.19, found 391.38. The activated E3 hapten (71)-DMF solution (20 mg/mL) was used to prepare the conjugates/immunogens in Table 7 as well as (74a)-(74e) conjugates.

### Example 8: Preparation of Protein Conjugates and Immunogens with Activated E3 Hapten (71)

Synthesis of E3-OVA conjugate (72) and E3-KLH immunogen (73a)-(73c)) (FIG. 5, Scheme 5): Proteins (OVA, KLH), were dissolved in PBS (100 mM, pH 8.0, 200 mM NaCl) at 5 mg/mL concentration. The protein solutions were cooled on an ice bath, then, the activated E3 hapten (71)-DMF solutions were added dropwise to each protein solution (Table 7, Column 2). The resulting reaction mixtures were stirred in a cold room overnight. The E3-OVA conjugate (72) was filtered-off and dialyzed with PB (2×1 L of 100 mM, pH 7.0+200 mM NaCl) then, with PB (4×1 L of 50 mM, pH 7.0). The E3-KLH immunogens (73a)-(73c) were dialyzed without filtration with PB (4×1 L of 50 mM, pH 7.0).

TABLE 7

| Conjugate ID | Hapten mol excess. | V [mL] \| |
|---|---|---|
| (E3 conjugates) | V_{DMF} at 20 mg/mL | conc [mg/mL] |
| 1 | 2 | 3 |
| MDMA-N-VAL-OVA | 25x \| 4.38 mg \| | 5.5 \| 3.18 |
| (E3-OVA (72)) | 0.214 mL | |
| MDMA-N-Val-KLH-250x | 250x \| 3.21 mg \| | 5.5 \| 3.63 |
| (E3-KLH (73a)) | 0.160 mL | |
| MDMA-N-Val-KLH-500x | 500x \| 6.41 mg \| | 3.33 \| 6.00 |
| (E3-KLH (73b)) | 0.320 mL | |
| MDMA-N-Val-KLH-1000x | 1000x \| 12.83 mg \| | 3.33 \| 6.00 |
| (E3-KLH (73c)) | 0.640 mL | |

### Example 9: Preparation and Activation of a Native G6PDH Enzyme Conjugate

Preparation of the native G6PDH enzyme conjugate (FIG. 6, Scheme 6): The native G6PDH enzyme emulsion (4.5 mL, 45 KU, 52.7 mg) was loaded onto a centrifuge tube and spun at 18,000 g, 4° C. for 18 min. The supernatant was disposed, and the white precipitate was dissolved in PB (50 mM, pH 7.9, 5 mL) and loaded onto a dialysis bag (10000 MWCO). The enzyme was buffer exchanged with PB (3×500 mL of 50 mM, pH 7.9) in a cold room (2-8° C.). A volume of 5.2 mL of enzyme solution with a concentration of 10.5 mg/mL was recovered. The enzyme solution was diluted with PB (200 μL of 50 mM, pH 7.9) to a conc. of 9.14 mg/mL (49.36 mg enzyme in total).

The enzyme solution was then cooled on an ice bath; then, Glucose-6-phosphate di-sodium salt (G6PDNa₂) 116 mg was added, the resulting mixture was swirled until dissolved (approximately 1 min); then, β-NADH 200 mg was added, the resulting reaction mixture was swirled until dissolved. The enzyme solution was then divided into two vials (~25 mg of enzyme each). The enzyme solutions were cooled on an ice bath.

Activation of enzyme (FIG. 6, Scheme 6): A solution of BrAcSu (10 mg/mL) in degassed DMF was prepared by dissolving BrAcSu (6.8 mg) in DMF (0.68 mL). The BrAcSu-DMF solution was cooled and was added dropwise and added as following:
To vial 1, BrAcSu-DMF solution (108 μL, 1.08 mg, 20 mol excess) was added dropwise over 1 min and to vial 2 BrAcSu-DMF solution (163 μL, 1.63 mg, 30 mol excess) was added dropwise over 1 min. The resulting reaction mixtures were allowed to warm up to 7° C. for 15 min; then, they were transferred to a cold room (2-8° C.) and stirred for 90 min. The reaction mixtures were then loaded onto dialysis bags (10000 MWCO) and buffer exchanged with PB (4×500 mL) in a cold room, then further buffer exchanged on Amicon Ultra-15 centrifugal filters (10000 MWCO, 4×15 mL) until no UV active substrates were detected in the waste by OD₂₈₀.
For the 20× BrAcSu reaction, a volume of 3.48 mL enzyme was recovered with conc. 5.53 mg/mL; for the 30× BrAcSu reaction a volume of 3.42 mL was recovered with a conc. 4.79 mg/mL.
The 20×BrAc-G6PDH enzyme (57a) was placed into vials 1-3 (7 mg of enzyme per vial), and the 30×BrAc-G6PDH enzyme (57b) was placed into vials 4-6 (7 mg of enzyme per vial) see Table 8, column 2. The enzyme vials were cooled on an ice bath.

### Example 10: Preparation of Protein Conjugates and Immunogens with E1 Reduced Hapten (52)

Synthesis of activated E1 hapten (52) Conjugates (FIG. 6, Scheme 6): E1 hapten (51a) (3.0 mg), which was prepared by route a, was dissolved in MeOH (143 μL) then sodium acetate buffer (25 mM, pH 4.5, 340 μL) was added, followed by TCEP·HCl solution (141 μL, from a 10 mg/mL TCEP·HCl-NaOAc buffer). The resulting reaction mixture was stirred for 2 h at rt to give activated E1 hapten (52). The hapten activation was confirmed by ESI-MS {m/z calcd, for [C₁₄H₂₁N₂O₃S]⁺ 297.13, found 297.13}. The activated E1 hapten (52) solution, having a concentration was 4.81 mg/mL, was added dropwise to the activated enzymes ((57a) (57b)) at 15 to 35 mol excess (Table 8, Column 3) on an ice bath. The resulting reaction mixtures were allowed to warm to 7° C. for 15 min; then, were placed in a cold room (2-8° C.) and stirred overnight (16 h). The resulting conjugates were purified on Sephadex® G50 M column [(CV=71 mL; diameter: 1.5 cm, height 40 cm, pre-equilibrated with PB (50 mM, pH 7.0)], filtered through Corning 0.20 μm filters, the concentration was estimated by OD₂₈₀. The concentrations and volumes of the recovered E1-G6PDH (58a, 58b, 58c, 59a, 59b, 59c) conjugates are provided in Table 8, Column 4.

TABLE 8

| | | Act. (51) | c [mg/mL] \| |
|---|---|---|---|
| Vial ID | v [mL] m [mg] | [mol x] \| v [μL] | v [mL] |
| 1 | 2 | 3 | 4 |
| 1 (58a) (20x BrAcSu) | 1.27 \| 7 | 15x \| 60 | 0.66 \| 9.80 |
| 2 (58b) 20x BrAcSu) | (of 5.53 | 20x \| 79.5 | 0.70 \| 10.40 |
| 3 (58c) (20x BrAcSu) | mg/mL) | 25x \| 99.3 | 0.64 \| 10.50 |
| 4 (59a) (30x BrAcSu) | 1.46 \| 7 | 20x \| 79.4 | 0.80 \| 9.00 |
| 5 (59b) (30x BrAcSu) | (of 4.80 | 30x \| 119.2 | 0.61 \| 6.70 |
| 6 (59c) (30x BrAcSu) | mg/mL) | 35x \| 139.0 | 0.78 \| 9.50 |

### Example 10: Preparation of Protein Conjugates and Immunogens with Activated E2 Hapten (61) and E3 Hapten (70)

Activation of E2 hapten (60) (FIG. 7B, Scheme 7b): E2 hapten (60) (1.6 mg, 0.0043 mmol) was dissolved in DMF (0.2 mL), then EDC·HCl-DMF solution (45 μL, 0.90 mg, 0.0047 mmol at 20 mg/mL) and SuOH-DMF solution (15 μL, 0.6 mg, 0.0052 mmol at 40 mg/mL) were added. The resulting reaction mixture was stirred at rt for 24 h, then heated to 40° C. for 6 h to give activated E2 hapten (61). The formation of the activated E2 hapten (61) was confirmed by ESI-MS m/z calcd. for [C₂₁H₂₃F₃N₂NaO₇]⁺ 495.14, found 495.40.

The remaining activated E3 hapten (71)-DMF (E3-DMF) solution prepared in Example 7 (FIG. 5, Scheme 5) was used for the preparation of E3-G6PDH conjugates (E3-G6PDH conjugates) (FIG. 7A, Scheme 7a). The activated E3 hapten (71)-DMF (100 μL at 20 mg/mL) was diluted with DMF (300 μL) to make a 5 mg/mL solution.

Enzyme preparation: G6PDH enzyme emulsion (6 mL, 60 KU, 70 mg) was loaded onto a centrifuge stirring tube and spun at 18,000 g, 4° C. for 30 min. The supernatant was disposed, and the resulting precipitate was dissolved under gentle mixing in 5 mL of Tris Buffer (55 mM, Tris, pH 8.0). The clear solution was divided into two portions.

E3-G6PDH (bioconjugation process (FIG. 7A, Scheme 7a): The first portion of G6PDH enzyme was buffer exchanged with Tris buffer (3×1 L of 55 mM Tris, pH 8.0 in a cold room (2-8° C.)), the concentration was then adjusted to 5 mg/mL using the Tris buffer. The enzyme was cooled on an ice-bath then G6PDNa₂ (68 mg) was added, the resulting mixture was stirred until all solids are dissolved (~40 sec.), then β-NADH (34 mg) was added and the resulting mixture was stirred until all solids are dissolved (30 sec-1 min.). A volume of 0.980 mL (5.0 mg enzyme) was placed in vials 1-5. (Table 9) These vials were placed on an ice bath; then, a volume of activated E3 hapten (71)-DMF solution (5 mg/mL), corresponding to 5 to 25 mol excess respective to enzyme, was added dropwise under gentle stirring to each vial (Table 9, column 3). The resulting reaction mixtures were allowed to warm-up for 10 min, then they were transferred to a cold room for 2 h, and then quenched with L-Lysine (1M, 10 mol excess respective to activated E3 hapten (71)) at rt for 15 min. The resulting conjugates were purified on a manually packed G50M Sephadex® column (CV=71 mL; diameter: 1.5 cm, height 40 cm) using a Tris buffer (55 mM Tris, pH 7.0). The concentration/volumes of recovered amounts E3-G6PDH conjugates (74a-74e) are presented in Table 9, columns 4 and 5.

E2-G6PDH bioconjugation process (FIG. 7B, Scheme 7b): The second portion of G6PDH enzyme was buffer exchanged with Tris buffer (55 mM, TRIS pH 7.5, 3×1 L in a cold room), the concentration was adjusted to 5 mg/mL using the Tris buffer. The enzyme was cooled on an ice-bath, then G6PDNa₂ (68 mg) was added, the resulting mixture was stirred until all solids are dissolved (~40 sec), then β-NADH (34 mg) was added, and the resulting mixture was stirred until all solids were dissolved (30 sec-1 min). A volume of 952 μL (4.9 mg enzyme) was placed in vials 6-10 (Table 9). These vials were placed on an ice-bath; then, a volume of activated E2 hapten (61)-DMF solution (5 mg/mL) corresponding to 5 to 25 mol excess respective to enzyme was added dropwise under gentle stirring to each vial. The resulting reaction mixtures were allowed to warm up for 10 min, then transferred to a cold room for 2 h, and then quenched with L-Lysine (1M, 10 mol excess respective to activated E2 hapten (61)) for 15 min at rt.

Trifluoroacetate deprotection (FIG. 7B, Scheme 7b): Each E2-G6PDH conjugate was loaded onto a dialysis bag (10,000 MWCO) and buffer exchanged with PB (50 mM, pH 11.4) in a cold room for 16 h, then with PB (50 mM, pH 8.4) for 7 days. The resulting E2-G6PDH conjugates are purified on a manually packed G50M Sephadex® column (CV=71 mL; diameter: 1.5 cm, height 40 cm) using Tris Buffer (55 mM, Tris pH 7.0). The concentration/volumes of the recovered E2-G6PDH conjugates (68a-68e) are presented in Table 9, columns 4 and 5.

TABLE 9

| | hapten | hapten-DMF | Conc | Vol |
|---|---|---|---|---|
| Conjugate ID | [mg] | sol. [μL] | mg/mL | [mL] |
| 1 | 2 | 3 | 4 | 5 |

E3-G6PDH (74) conjugates

| | | | | |
|---|---|---|---|---|
| E3-G6PDH (74a) (5X) | 0.068 | 13.6 | 0.46 | 8.87 |
| E3-G6PDH (74b) (10X) | 0.137 | 27.2 | 0.49 | 8.91 |
| E3-G6PDH (74c) (15X) | 0.204 | 40.8 | 0.50 | 8.90 |
| E3-G6PDH (74d) (20X) | 0.275 | 55.0 | 0.57 | 8.33 |
| E3-G6PDH (74e) (25X) | 0.343 | 69.0 | 0.53 | 9.71 |

E2-G6PDH (68) conjugates

| | | | | |
|---|---|---|---|---|
| E2-G6PDH (68a) (5x) | 0.102 | 21 | 0.45 | 8.87 |
| E2-G6PDH (68b) (10x) | 0.206 | 42 | 0.42 | 9.86 |
| E2-G6PDH (68c) (15x) | 0.304 | 63 | 0.46 | 9.10 |
| E2-G6PDH (68d) (20x) | 0.272 | 84 | 0.49 | 8.33 |
| E2-G6PDH (68e) (25x) | 0.340 | 105 | 0.49 | 8.17 |

### Example 11: Synthesis of E4 Hapten (81)

Synthesis of E4 hapten (81) (FIG. 8, Scheme 8): Piperonyl methyl ketone (PMK) (79) (32.14 mg, 0.168 mmol) was suspended in MeOH (0.4 mL); then, NaOAc (68 mg) was added followed by the aminooxyaminobromoacetate linker (80) (56 mg, 0.168 mmol). The resulting reaction mixture was stirred at rt overnight, then heated to 40° C. for 6 h. The volatiles were removed on a rotatory evaporator to give a pale-yellow precipitate which was suspended in EtOAc (20 mL) and washed with a saturated monobasic phosphate solution (pH 4.3, 3×10 mL). The organic layer was dried over Na₂SO₄; the solvent was removed on a rotatory evaporator to give a yellow oil, which was then purified on a Biotage LC (Hexanes/EtOAc). Fractions containing the useful product were pooled out, concentrated on a rotatory evaporator and further dried on an oil pump to give 60 mg of product (0.145 mmol) for E4 hapten (81) as a yellow oil in 86% yield as a mixture of -syn/-anti isomers in 1:2 molar ratio. ESI-MS m/z calcd, for [C₁₆H₂₁BrN₃O₅]⁺ 414.07, 416.06, found 414.20, 416.21. ¹H NMR (600 MHz, CD₃CN) 7.03 (brs, 1H), 6.86 (brs, 1H), 6.86-6.70 (m, 3H), 5.93-5.92 (m, 2H), 4.41 (brs, 2H), 3.78 (m, 1.6H), 3.67 (s, 0.4H), 3.37 (s, 1.3H), 3.33-3.29 (m, 3.7H), 1.82 (s, 2H), 1.77 (s, 1H). ¹³C NMR (150 MHz, CD₃CN) 171.36, 171.25, 167.72, 160.24, 159.76, 148.97, 148.93, 147.62, 147.41, 131.67, 131.42, 130.96, 123.36, 123.23, 123.17, 110.57, 110.51, 110.25, 109.24, 109.20, 109.10, 102.37, 102.31, 73.51, 73.46, 43.44, 41.99, 40.73, 40.63, 39.51, 35.90, 30.03, 26.44, 20.05, 14.40.

### Example 12: Preparation of Protein Conjugates and Immunogens with E4 Hapten (81)

E4-rG6PDH (82) bioconjugation process (Scheme 8): Recombinant G6PDH (3KG6PDH) enzyme (10.2 mg) was loaded onto a manually packed G50M Sephadex® column (CV=71 mL; diameter: 1.5 cm, height 40 cm) pre-equilibrated with PB (50 mM, pH 7.3, 1 mM EDTA). 3KG6PDH is a mutant G6PDH from E. coli having a cystine at amino acid position 52. Both the wild-type G6PDH and 3KG6PDH are described in U.S. Pat. No. 6,455,288 (Benjamin, et al.), which is incorporated herein by reference in its entirety. After column, the recovered enzyme was concentrated to 5 mg/mL on a Amicon Ultra-15 centrifugal filter unit (1.19 mL). The enzyme was cooled on an ice-bath, blanketed with N₂, then DTT solution (19.1 μL, 0.5M) is added. The resulting mixture was rocked in a cold room (2-8° C.) overnight (16 h) to produce the reduced enzyme. The reduced enzyme was buffer exchanged on a manually packed G50M Sephadex® column (CV=71 mL; diameter: 1.5 cm, height 40 cm) with PB (1 L of PB5+0.025 mM DTT). After column, the reduced enzyme was concentrated to 4.17 mg/mL using a centrifugal tube.

E4 hapten (81) (3.93 mg, 0.0095 mmol) was dissolved in DMF (390 μL) to make a 10 mg/mL solution. E4 hapten (81)-DMF solution (202 μL), corresponding to 60 mol excess respective to rG6PDH was added dropwise to the reduced enzyme. The resulting reaction mixture was blanketed with N₂ and rocked in a cold room (2-8° C.) for 16 h. The conjugate was then purified on manually packed G50M Sephadex® column (CV=71 mL; diameter: 1.5 cm, height 40 cm) equilibrated with PB (50 mM PB, pH 7.0). A volume of 11.26 mL of E4-rG6PDH conjugate (82) with a concentration of 0.67 mg/mL was recovered.

### Example 13: Synthesis and Activation of E6 Hapten (19)

Preparation of compound 15 (FIG. 9A, Scheme 9a): PMK (1.00 g, 6.66 mmol) and methyl-4-nitrobutirate (1.27 g, 1.3 eq., 8.66 mmol) were added to a mixture of formic acid:aminoethanol (5 mL 1/1 molar ratio). The resulting reaction mixture was heated to 60° C. for 7 days. [TLC analysis EtOAc/Hexanes 4/3 v/v R_{f prod}~0.6 as yellow spot]. Ethyl acetate (30 mL) was added and the organic layer was washed with water (2×15 mL). The organic layer was then concentrated to give a yellow oil, which was purified on a Biotage LC (Hexanes/EtOAc); the peak recorded at 360 nm was collected. The volatiles were concentrated in vacuo, traces of methyl-4-nitrobutirate are distilled-off at 140° C. under oil-pump vacuum, to give 1.40 g (5.018 mmol) of compound 15 as a yellow oil in 75% yield. ¹H NMR (600 MHz, CD₃CN) 8.02 (s, 1H), 7.08-7.05 (m, 2H), 6.93 (d, J=8.03 Hz, 1H), 6.041 (s, 2H), 3.63 (s, 3H), 3.15-3.12 (m, 2H), 2.64-2.61 (m, 2H). ESI-MS m/z calcd, for [C₁₃H₁₄NO₆]⁺ 280.08; parent peak not stable under the LC conditions, ret. time 6.290 min (with UV absorption at 360 nm).

Preparation of compound 16 (FIG. 9A, Scheme 9a): In an oven-dried flask equipped with a magnetic stir bar and a condenser compound 15 (400 mg, 1.433 mmol) was dissolved in anhydrous THF (10 mL). The resulting reaction mixture was blanketed with Argon, then, LiAlH₄/THF (10 mL, 7 eq., 1M) was added dropwise through a septum. The resulting reaction mixture was then heated to 60-70° C. for 20 h under Argon positive pressure. The reaction mixture was then allowed to cool to rt for 30 min, then, on an ice-bath and quenched by slowly adding NaOH (400 μL, 3.75N) followed by water (1.20 mL) under inert gas flow (argon). The formed LiAl(OH)₄—NaOH precipitate separated and it was filtered-off. The precipitate was further washed with THF (~20 mL), the combined extracts were concentrated on a rotatory evaporator to ~2 mL {ESI-MS m/z for [C₁₂H₁₈NO₃]⁺ 224.13, found 224.08}. NEt₃ (0.340 mL, 1.7 eq) was added followed by BoczO-THF solution (1.2 eq. 0.860 mL 2M). The resulting reaction mixture was stirred at rt for 4 h. The product was extracted in DCM (40 mL), washed with water (2×20 mL) and dried under vacuum to give 460 mg (98% yield) of compound 16. ESI-MS m/z calcd, for [C₁₇H₂₅NnaO₅]⁺ 346.16, found 346.21, ret. time 3.864 min.

Preparation of compound 17 (FIG. 9A, Scheme 9a): Compound 16 (460 mg, 1.42 mmol) was dissolved in dichloromethane (3 mL), then NEt₃ (359 μL, 259 mg, 1.8 eq.) was added and the resulting reaction mixture was cooled on an ice-bath. MsCl (163 μL, 1.5 eq.) was added and the resulting reaction mixture was stirred for 1 h at rt. The reaction mixture was concentrated on a rotatory evaporator, the formed residue was dissolved in dichloromethane (20 mL) and washed with water (2×10 mL). The solvent was removed in vacuum to give the crude intermediate which was suspended in THF (1.5 mL). An emulsion of potassium thioacetate in DMF (177 mg, 1.56 mmol, 1.1 eq. in 1 mL DMF) was added followed by K₂CO₃ (98 mg, 0.5 eq.). The resulting reaction mixture was heated to 80° C. for 2 h. The volatiles were removed in vacuum to yield a crimson solid which was purified on Biotage LC (Hexanes/Ethyl Acetate). Fractions containing the useful product are pooled out, concentrated in vacuo to give 300 mg (0.787 mmol) of compound 17 in 55% yield. ESI-MS m/z calcd. for [C₁₉H₂₇NnaO₅S]⁻ 404.15, found 404.20, ret., time 5.035, 5.356 mins (mixture of isomers).

Preparation of compound 18 (FIG. 18, Scheme 9a): Compound 17 (300 mg, 0.787 mmol) was dissolved in MeOH (1 mL), then NaOH (0.31 mL, 4 eq., 10N) was added. The resulting reaction mixture was stirred at rt for 2 h. The volatiles were removed on a rotatory evaporator. The resulting oil was dissolved in DCM (30 mL) and washed with DI water (2×15 mL). The organic layer was concentrated in vacuo to give (238 mg, 0.354 mmol) of compound 18 in 90% yield-dimerization). ESI-MS m/z for [C₃₄H₄₈N₂NaO₅S₂]⁺ 699.27, found 699.49.

Preparation of E6 hapten (19) (FIG. 9A, Scheme 9a): Compound 18 (238 mg, 0.354 mmol) was dissolved in MeOH (7 mL). HCl (300 μL, 12N, 10 eq.) was added and the resulting reaction mixture was heated to 60° C. for 6 h. The volatiles were removed in vacuo, the resulting oily product lyophilized overnight to give 140 mg (0.294 mmol) of E6 hapten (19) in 83% yield as brown hydroscopic crystals. ESI-MS m/z calcd, for [C₂₄H₃₃N₂O₄S₂]⁺ 477.19, found 477.27, ret. time 2.399 min. ¹H NMR (600 MHz, MeOD-d₄) 6.79 (d, J=7.88 Hz, 2H), 6.77 (d, J=1.58 Hz, 2H), 6.72 (dd, J=7.88, 1.60 Hz, 2H), 5.93 (s, 4H), 3.41 (qui, J=6.53 Hz, 2H), 2.87 (dd, J=14.12, 7.17 Hz, 2H), 2.82 (dd, J=14.11, 7.02 Hz, 2H), 2.69-2.66 (m, 4H), 1.86-1.81 (m, 2H), 1.77-1.70 (m, 6H). ¹³C NMR (150 MHz, MeOD-d₄) 148.29, 147.10, 129.13, 122.25, 109.02, 108.16, 101.10, 52.62, 38.14, 37.10, 30.69, 24.24.

Activation of E6 hapten (19) (FIG. 9A, Scheme 9a): The E6 hapten (19) 2.80 mg (0.51 mmol) was dissolved in MeOH (140 μL), NaOAc buffer (307 μL, 0.25 M) was added, followed by TCEP·HCl (113 μL, 1.13 mg, 10 mg/mL in NaOAc buffer). The resulting reaction mixture was blanketed with Argon and stirred at rt for 16 h to give activated compound 20. The formation of the activated E6 hapten (20) was confirmed by ESI-MS, however, additional TCEP·HCl solution was necessary to complete the reaction. The final concentration of this compound 20 was 4.37 mg/mL. ESI-MS m/z calcd, for [C₁₂H₁₈NO₂S]⁺ 240.11, found 240.13 ret time 2.334 and 2.505 min (mixture of isomers).

### Example 14: Activation of E5 Hapten (9)

Activation of E5 hapten (9) (FIG. 9B, Scheme 9b): E5 hapten (9) (3 mg (0.5 mmol)) was dissolved in MeOH (150 μL), NaOAc buffer (337 μL, 0.25M) was added, followed by TCEP·HCl (1.13 mg, 113 μL 10 mg/mL in NaOAc buffer). The resulting reaction mixture was blanketed with Argon and stirred at rt for 16 h to give activated compound 22. The formation of activated E5 hapten (22) was confirmed by ESI-MS; however, additional TCEP·HCl solution was necessary to complete the reaction. The final concentration of activated E5 hapten (22) was 4.41 mg/mL. ESI-MS m/z calcd, for [C₁₄H₂₂NO₂S]⁺ 268.14, found 268.12 ret. Time 2.228 min and 2.702 min (mixture of two isomers).

### Example 15: Preparation of Protein Conjugates and Immunogens with Activated E6 Hapten (20) and Activated E5 Hapten (22)

Enzyme preparation (FIG. 9C, Scheme 9C): The G6PDH enzyme emulsion (6 mL, 60 KU, 70 mg) was loaded onto a centrifuge stirring tube and spun at 18,000 g, 4° C. for 30 min. The supernatant was disposed, and the precipitate was dissolved in PB (50 mM, pH 7.9) and loaded onto a dialysis bag. The enzyme was buffer exchanged with PB (3×1000 mL of 50 mM, pH 7.9), then, the concentration was adjusted to 20 mg/mL using PB8. The enzyme was then cooled on an ice-bath, G6PDNa₂ (70 mg) was added, the resulting mixture was mixed until all solids were dissolved (~40 sec), then, β-NADH (140 mg) was added, the resulting reaction mixture was mixed until all solids were dissolved (30 sec-1 min). The enzyme solution was then divided into two vials (~33 mg enzyme each). A fresh solution of BrAcSu (5 mg) in degassed DMF (0.4 mL) was prepared.

Preparation of 20× BrAcG6PDH (23a) (FIG. 9C, Scheme 9C): The first vial, containing the G6PDH enzyme (33 mg, 1.65 mL), was placed on an ice bath and cooled to 7° C., then BrAcSu-DMF (72 μL, 20 mol. excess of 20 mg/mL solution) was added dropwise. The resulting reaction mixture was allowed to warm up to rt and stirred for 90 min.

Preparation of 30× BrAcG6PDH (23b) (FIG. 9C, Scheme 9): To the second vial containing the G6PDH enzyme (33 mg, 1.65 mL), BrAcSu-DMF solution (107 μL, 30 mol. Excess) was added dropwise, following the same reaction conditions as for 20× BrAcG6PDH.

The activated enzymes were loaded onto a pre-equilibrated G25M Sephadex® column (CV=71 mL; diameter: 1.5 cm, height 40 cm) and buffer exchanged with PB (50 mM, pH 7.9). The concentration was then adjusted to approx. 10 mg/mL.

The activated enzyme solutions were placed in 12 vials (5.3 mg enzyme per vial) and cooled on an ice bath (4-7° C.), then, the volumes of activated E6 hapten (20)-DMF and activated E5 hapten (22)-DMF solutions shown in Table 4, column 4 are added dropwise. After the hapten addition, each reaction was allowed to warm up to room temperature for 15 mins, then, placed in a cold room and stirred for an additional 90 mins. Each conjugate was then purified on G25M Sephadex® column (CV=71 mL; diameter: 1.5 cm, height 40 cm), using PB (50 mM PB, pH 7.0). The concentration/volume of each recovered E5-G6PDH conjugate (24a-24c, 26a-26c) and E6-G6PDH (25a-c, 27a-c) is presented in Table 10, column 5.

TABLE 10

| | | | Compound | | Recovered | |
|---|---|---|---|---|---|---|
| | | | 20/22Hapten | | conjugate | |
| | Activated | Hapten | Mass | vol | | vol |
| Conj. ID | BrAcG6PDH | Mol x | [mg] | [μL] | c.[mg/mL] | [mL] |

| | | | | |
|---|---|---|---|---|
| 1 | 2 | 3 | 4 | 5 |

E5-G6PDH (22) conjugates

| | | | | | | |
|---|---|---|---|---|---|---|
| 24a | 20x | 15 | 0.28 | 63 | 0.46 | 11.5 |
| (BD109886773-1) | BrAcG6PDH | | | | | |
| 24b | | 20 | 0.37 | 84 | 0.43 | 12.0 |
| (BD109886773-2) | | | | | | |
| 24c | | 25 | 0.46 | 112 | 0.49 | 12.0 |
| (BD109886773-3) | | | | | | |
| 26a | 30x | 20 | 0.37 | 84 | 0.48 | 12.0 |
| (BD109886773-4) | BrAcG6PDH | | | | | |
| 26b | | 30 | 0.56 | 126 | 0.24 | 11.5 |
| (BD109886773-5) | | | | | | |
| 26c | | 35 | 0.65 | 157 | 0.15 | 14.5 |
| (BD109886773-6) | | | | | | |

E6-G6PDH (20) conjugates

| | | | | | | |
|---|---|---|---|---|---|---|
| 25a | 20x | 15 | 0.26 | 59 | 0.51 | 9.50 |
| (BD109886773-7) | BrAcG6PDH | | | | | |
| 25b | | 20 | 0.34 | 78.51 | 0.45 | 13.0 |
| (BD109886773-8) | | | | | | |
| 25c | | 25 | 0.43 | 85.77 | 0.50 | 10.5 |
| (BD109886773-9) | | | | | | |
| 27a | 30x | 20 | 0.34 | 78.51 | 0.52 | 10.5 |
| (BD109886773-10) | BrAcG6PDH | | | | | |
| 27b | | 30 | 0.51 | 117.77 | 0.29 | 14.5 |
| (BD109886773-11) | | | | | | |
| 27c | | 35 | 0.60 | 120.1 | 0.51 | 9.50 |
| (BD109886773-12) | | | | | | |

### Example 16: Production of Anti-Ecstasy Class Polyclonal Antibodies

Three groups of New Zealand White rabbits (5 per group) received 3 subcutaneous injections at four-week intervals of 250-500 μg/dose/animal of one of E1-KLH (56), E2-KLH (66), or E3-KLH (73). Additionally, female Balb/c, Swiss Webster, and A/J mice (minimum age of 12 weeks) were immunized with 3 intraperitoneal injections of 10⁻²⁰ μg/dose/animal of one of E1-KLH (56), E2-KLH (66), or E3-KLH (73) (10 mice/strain/immunogen) at 1-month intervals. For all groups, the immunogens were emulsified in CFA for the first dose and in IFA for the subsequent two doses. The animals were bled one week after the last injection. Antisera titers to E1-OVA (55), E2-OVA (65), and E3-OVA (72) were estimated by an indirect ELISA assay (respective pre-immune sera were used as negative controls). E1-OVA, E2-OVA, and E3-OVA are identical to E1-KLH, E2-KLH, and E3-KLH, respectively, except that KLH is replaced with OVA. Immune sera at appropriate dilutions were tested for binding with free MDMA or MDA in competitive ELISA.

### Indirect ELISA

All ELISA steps were performed at rt. Wells of a Nunc Maxi-Sorp™ flat-bottom ELISA plates (ThermoFisher Scientific, Waltham, MA) were coated with 50 μL E1-OVA (55), E2-OVA (65), and E3-OVA (72) at a concentration of 1 μg/mL in PBS for 1 h. The plates were flicked dried and remaining binding sites were blocked with 200 μL per well of blocking solution (0.5% casein, 0.05% (v/v) Tween 20 in PBS) for one hour. Plates were washed six times with MilliQ water containing 0.05% (v/v) Tween 20 on Biotek 405 LS plate washer equipped with microplate Biostacker 3 (Biotek, Winooski, VT). 50 μL of serially diluted antibody samples (serial dilutions of serum, hybridoma supernatant, or monoclonal antibodies in PBS) were added to each well and incubated for one hour. Unbound antibodies were removed by washing as described above, and 50 μL of secondary goat anti-rabbit IgG HRP or goat anti-mouse IgG (Fc)-HRP conjugate (ThermoFisher Scientific, USA), diluted 1:3,000 in the blocking solution was added to each well for detection bound rabbit or mouse antibody, respectively. Plates were incubated for one hour, washed and 100 μL of TMB substrate (Moss, Pasadena, MD) was added for 15 min to determine bound peroxidase. Optical density (OD) of the samples at 650 nm was measured using an ELISA plate reader (Molecular Devices LLC., San Jose, CA). The titer of each serum sample was designated as the maximum dilution that yielded at least twice the absorbance of the same dilution of the nonimmune control serum.

### Competitive ELISA

Competitive ELISA was used to assess the presence of anti-Ecstasy-class antibodies in animal sera, hybridoma supernatants, and monoclonal antibody (mAb) samples as well as to estimate the level of antibody cross reactivity to amphetamine (Amph) and methamphetamine (mAmph). Antisera and antibody samples were used at the dilution giving a response of about OD=1 at 650 nm (OD₆₅₀ nm) in an indirect ELISA. Hybridoma supernatants were diluted 1:2 in PBS. (±)-MDMA, (±)-MDA, (±)-amphetamine, and (±)-methamphetamine were used as inhibitors (Cerilliant Corporation, Round Rock, TX). Microtiter ELISA plates were coated and blocked as described above for indirect ELISA. The inhibitors were serially diluted in PBS. 25 μL of the diluted inhibitor was mixed with 25 μL of fixed appropriate antiserum or antibody sample dilution. The mixture was incubated over the E1-OVA (55) solid phase for 1 h. The plates were washed as described above and the bound antibodies were detected with anti-rabbit or anti-mouse IgG HRP-conjugates and TMB substrate as described above.

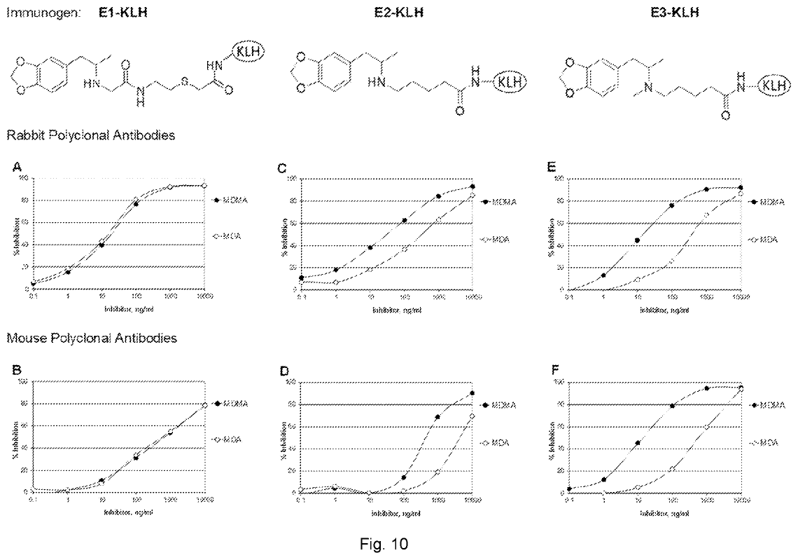

After receiving 3 injections of E1-KLH (56), E2-KLH (66), or E3-KLH (73), rabbits and mice developed high antibody titers (>1:100,000) not only to corresponding ovalbumin conjugate but to all three E1-OVA (55), E2-OVA (65), and E3-OVA (72) antigens (data not shown). The reactivities of the polyclonal antibodies toward MDMA and MDA were assessed by competitive ELISA. Antigen-binding properties of rabbit and mouse polyclonal antibodies generated in response to E1-KLH (56), E2-KLH (66) and E3-KLH (73) immunogens and tested on E1-OVA (55) coated plates are compared in FIG. 10. All groups of immunized animals developed MDMA/MDA-specific response. The inhibition pattern of each polyclonal antibody was not influenced by ovalbumin antigen used for coating ELISA plates (data not shown) and depends only on the immunogen structure. While antisera of all E2-KLH (FIGS. 10C and 10D) and E3-KLH (FIGS. 10E and 10F) immunized animals preferably bind MDMA over MDA, several E1-KLH immunized rabbits (2 out of 5) and mice (6 out of 30) produced polyclonal antibodies with unique ability to bind both compounds equally well (FIGS. 10A, and 10B) in 10,000-0.1 ng/mL range of drug concentration. Thus, it was demonstrated that animal immunization with E1-KLH reproducibly generates polyclonal antibodies recognizing MDMA and MDA with the same efficiency.

### Example 17: Development of MDMA/MDA-Specific Monoclonal Antibodies

This example illustrates the development of MDMA/MDA-specific monoclonal antibodies (mAbs) with equal sensitivity to both compounds which can be used in Ecstasy-class drugs specific assay.

Mice immunized with E1-KLH (56) that produced polyclonal antibodies with equal sensitivity to both MDA and MDMA were selected for generation of monoclonal antibodies. One month following the third injection of E1-KLH (56), the mice received an IP prefusion boost of 10 μg/dose/mouse of E1-KLH (56) in PBS. Three days later, the mice were sacrificed by cervical dislocation. Immune spleens were aseptically harvested and homogenized using Potter-Elvehjem glass tissue grinder with Teflon pestle (Sigma-Aldrich, St. Louis, MO). The resulting splenocyte suspensions were washed twice in 30 mL of cold serum-free IMDM containing L-glutamine (IMDM, Mediatech Inc, 10⁻⁰¹⁶-CM) by centrifugation at 400 g for 5 min at 4° C. Viable splenocytes were counted by trypan blue exclusion, resuspended at 1-2×10⁸ cells/mL in freezing medium (90% (v/v) heat-inactivated FBS, 10% (v/v) tissue culture grade DMSO (Sigma-Aldrich, St. Louis, MO)), transferred to cryovials (1 ml/vial) and frozen in liquid nitrogen.

The frozen immune splenocytes were later thawed and fused with murine P3X63Ag8.653 myeloma cells (ATCC CRL-1580) in the presence of PEG1500 (MilliporeSigma, Burlington, MA). A cryovial with frozen murine splenocytes was removed from liquid nitrogen storage and placed in 37° C. water bath for 2-3 min. The thawed cells were transferred to 30 mL of serum-free IMDM pre-warmed to 37° C. and centrifuged at 400 g for 5 min at 25° C. The supernatant was decanted, and the cell pellet was resuspended in 30 mL of the same medium.

P3X63Ag8.653 myeloma cells grown in IMDM containing L-glutamine and 10% (v/v) heat-inactivated FBS were counted by trypan blue exclusion, washed twice in pre-warmed IMDM and added to the tube with mouse splenocytes at 1:3 ratio of viable splenocytes count. The cells mixture was centrifuged as described above and supernatant was decanted. The tube with the cells mixture was gently tapped to loosen the pelleted cells. One mL of pre-warmed PEG solution was added to the cells drop-by-drop during 1 min while gently mixing. The cells were incubated at room temperature for 1 min, then pre-warmed serum-free IMDM was added to the tube as follows: 1 mL during 1 min, and another 15 mL during 3 min. Then the cell suspension was centrifuged, and the supernatant was decanted. The fused cells were resuspended in 200 mL of selective medium containing hypoxanthine, aminopterin and thymidine (HAT) (IMDM with 20% (v/v) heat-inactivated FBS and 50×HAT supplement (Sigma-Aldrich, St. Louis, MO) diluted 1:50), and pipetted at 200 μL/well on 10 sterile, covered 96-well tissue culture plates (Corning, Corning, NY). The plates were incubated in CO₂ incubator at 37° C., 5% CO₂ and >80% relative humidity. After a 10⁻¹⁴ day cell cultivation, 100 μL of the supernatant was withdrawn from each well for screening and replaced with 120 μL of HT medium (hypoxanthine and thymidine and consisting of IMDM with 20% (v/v) FBS and 50× HT supplement (Sigma-Aldrich, St. Louis, MO) diluted 1:50). Then hybridomas cultivation was continued at conditions described above.

The supernatants were screened in a competitive ELISA for the presence of anti-ecstasy compound monoclonal antibodies (mAbs) as follows. 25 μL of the hybridoma supernatant was mixed with equal volume of PBS alone or containing 20 μg/mL inhibitor (MDA or MDMA) were incubated for 1 h in the wells of E1-OVA (55) coated ELISA plates. The plates were washed and the presence of bound mAbs were detected using goat anti-mouse IgG (Fc)-HRP conjugate and TMB substrate as described above. The level of inhibition of mAb/E1-OVA (55) binding in the presence of the inhibitor was estimated using a Percent (%) Inhibition calculated by the below formula:

$$\% Inhibition = ( 1 - \frac{{OD}_{650 nm} with Inhibitor}{{OD}_{650 nm} w / o Inhibitor} ) \cdot 100 \% ,$$

where “OD_{650 nm} w/o Inhibitor” is the binding response of the antibody sample diluted in PBS alone (with no inhibitor), and the “OD_{650 nm} with Inhibitor” is the binding response of the same antibody sample in the presence of 10 μg/mL of inhibitor (i.e., MDA or MDMA).

Hybridoma supernatants demonstrating strong binding to E1-OVA (55) (≥2 OD_{650 nm}) and similar inhibition (±5%) in the presence of 10 μg/mL of MDMA and MDA were considered positive, and corresponding hybridomas were subcloned twice by limiting dilution. Three positive single sub-clones per hybridoma were frozen to create a cell bank, and one was expanded for mAb production. Examples of the selected hybridomas producing MDMA/MDA-specific mAb are shown in Table 11.

TABLE 11
Hybridoma producing MDMA/MDA-specific monoclonal antibodies

| | | E1-OVA (55) Binding, OD_{650 nm} | | | % Inhibition by 10 | |
|---|---|---|---|---|---|---|
| | mAb | w/o | | | μg/mL of | |
| mAb | isotype | inhib | +MDA | +MDMA | MDA | MDMA |
| 178F 4H5 | IgG2a, κ | 2.948 | 0.109 | 0.076 | 95 | 96 |
| 178F 4C12 | IgG1, κ | 2.836 | 0.306 | 0.216 | 87 | 91 |
| 178H 2A7 | IgG1, κ | 2.900 | 0.114 | 0.095 | 97 | 96 |
| 178H 4B1 | IgG2b, κ | 3.164 | 0.105 | 0.093 | 97 | 98 |
| 178K 1B2 | IgG2b, κ | 4.000 | 0.730 | 0.667 | 82 | 83 |
| 178K 1F4 | IgG2b, κ | 3.012 | 0.254 | 0.359 | 93 | 88 |
| 178K 1E11 | IgG2b, κ | 3.390 | 0.316 | 0.301 | 91 | 91 |
| 178K 2B7 | IgG2b, κ | 3.497 | 0.429 | 0.374 | 88 | 89 |
| 178K 3C8 | IgG1, κ | 2.389 | 0.069 | 0.071 | 97 | 97 |
| 178K 4E11 | IgG2b, κ | 1.930 | 0.091 | 0.088 | 95 | 95 |
| 178K 5B11 | lgG1, κ | 2.893 | 0.057 | 0.073 | 98 | 97 |

The anti-ecstasy class mAbs were purified from clarified and filtered hybridoma culture supernatants on a recombinant Protein A Sepharose Fast Flow (GE Life Sciences, Boston USA), dialyzed against PBS containing 0.02% sodium azide and stored at 4° C. The purified mAbs were tested for MDA and MDMA reactivity as well as amphetamine (Amph) and methamphetamine (mAmph) cross reactivity in a competitive assay.

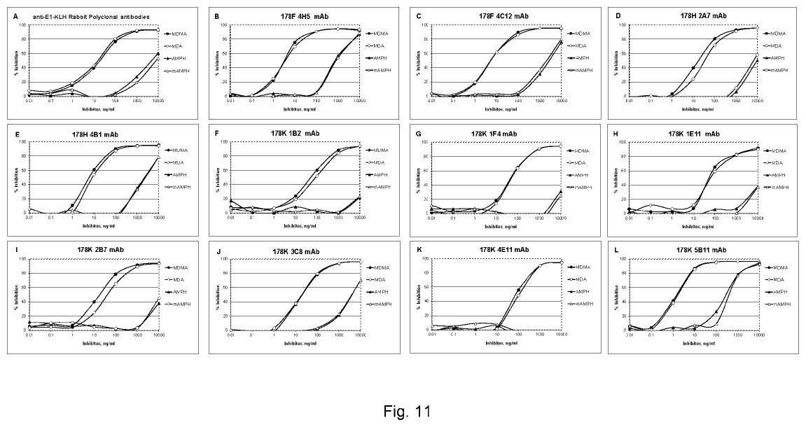

As shown in FIG. 11, anti-E1-KLH (56) rabbit polyclonal (FIG. 11.3A) and mouse monoclonal (FIG. 11B-L) antibodies are specific to Ecstasy class drugs and distinguish them from amphetamines. The sensitivity of the mAbs to MDMA and MDA in competitive ELISA varies from 10 to 0.1 ng/mL. All tested antibodies do not show any significant cross reactivity in the presence up to 100 ng/ml of amphetamines, and six of them (178K 2A7, 178K 1B2, 178K 1F4, 178K 1E11, 178K 2B7 and 178K 4E11)—in the presence of 1,000 ng/ml of Amph or mAmph (FIGS. 11D, 11F-11I, and 11K, respectfully). Anti-E1-KLH (56) rabbit polyclonal (FIG. 11A) and 7 mouse mAbs (FIGS. 11B, 11C, 11G, 11H, 11J, 11K, and 11L, respectively) demonstrate identical sensitivity to MDMA and MDA in 10,000-0.1 ng/mL range of drug concentration in competitive ELISA. Another four mAbs (178H 2A7, 178H 4B1, 178K 1B2 and 178K 2B7) bind MDMA stronger than MDA (FIGS. 11D-11F, and 11I, respectively) but the difference does not exceed 20% within the same range of drug concentration. Thus, the rabbit polyclonal and mouse monoclonal antibodies generated in response to E1-KLH (56) immunogen have a unique ability to bind both MDMA and MDA compounds with the same efficiency and distinguish Ecstasy class drugs from structure related compounds such as amphetamine and methamphetamine.

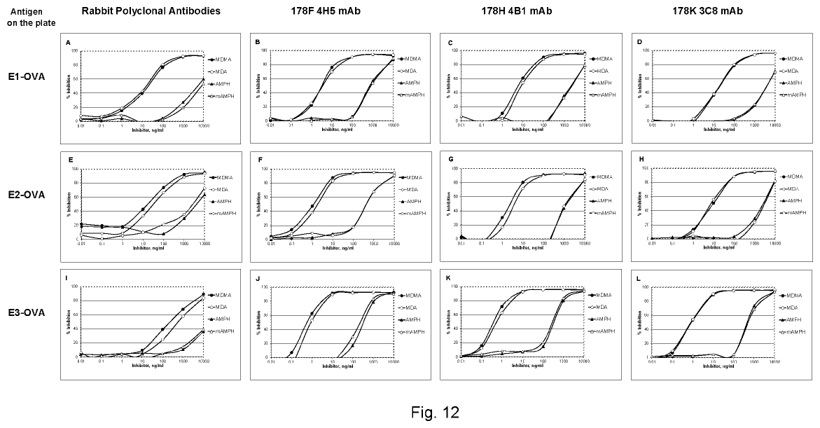

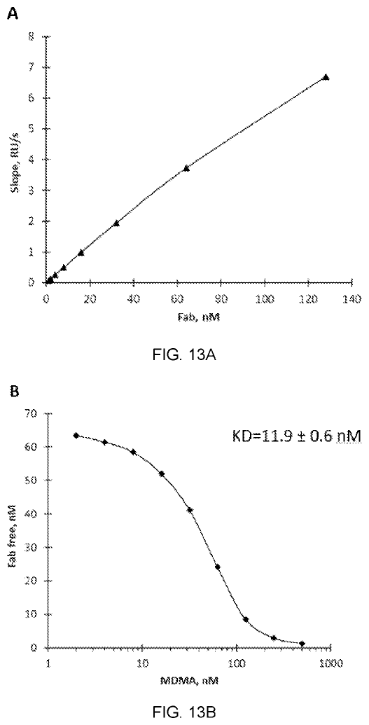

The results of competitive assays performed for E1-KLH (56)-specific antibodies on E1-OVA (55) (FIGS. 12A-12D), E2-OVA (65) (FIGS. 12E-12H) and E3-OVA (72) (FIGS. 121-12L) coated plates demonstrate that the antigen-binding properties of the antibodies do not depend on the antigen used in ELISA but depends only on the primary structure of the corresponding immunoglobulin heavy and light chains.

### Example 18: Monoclonal Antibody Sequencing

The hybridomas produced in Example 17 were used to sequence the corresponding monoclonal antibody heavy and light chains. Tables 12-22 show the sequence identifier for the amino acids that comprise the heavy and light chains of the specific mAbs reported herein. These sequences include DNA sequences of both heavy and light chains, amino acid sequences of heavy and light chains, amino acid sequences of heavy and light chain variable regions, and amino acid sequences of the three complementarity determining regions (CDR1, CDR2, and CDR3) for both heavy and light chain variable regions.

TABLE 12
Sequence Identifiers for Nucleotide and Amino
acid sequences of the Anti-Ecstasy 178F 4H5 mAb

| | SEQ ID NO: of | SEQ ID NO: of |
|---|---|---|
| | Amino Acid | DNA |
| Sequence | Sequence | Sequence |
| Heavy Chain | 81 | 143 |
| Heavy Chain CDR1 | 5 | 191 |
| Heavy Chain CDR2 | 13 | 197 |
| Heavy Chain CDR3 | 27 | 210 |
| Light Chain | 105 | 169 |
| Light Chain CDR1 | 35 | 216 |
| Light Chain CDR2 | 44 | 224 |
| Light Chain CDR3 | 49 | 228 |
| Heavy Chain Variable Region | 69 | 119 |
| Light Chain Variable Region | 93 | 156 |

TABLE 13
Sequence Identifiers for Nucleotide and Amino acid
sequences of the Anti-Ecstasy 178F 4C12 mAb

| | SEQ ID NO: of | SEQ ID NO: of |
|---|---|---|
| | Amino Acid | DNA |
| Sequence | Sequence | Sequence |
| Heavy Chain | 80 | 142 |
| Heavy Chain CDR1 | 6 | 192 |
| Heavy Chain CDR2 | 14 | 198 |
| Heavy Chain CDR3 | 26 | 209 |
| Light Chain | 104 | 168 |
| Light Chain CDR1 | 36 | 217 |
| Light Chain CDR2 | 44 | 224 |
| Light Chain CDR3 | 50 | 229 |
| Heavy Chain Variable Region | 68 | 118 |
| Light Chain Variable Region | 92 | 155 |

TABLE 14
Sequence Identifiers for Nucleotide and Amino
acid sequences of the Anti-Ecstasy 178H 2A7 mAb

| | SEQ ID NO: of | SEQ ID NO: of |
|---|---|---|
| | Amino Acid | DNA |
| Sequence | Sequence | Sequence |
| Heavy Chain | 82 | 144 |
| Heavy Chain CDR1 | 7 | 193 |
| Heavy Chain CDR2 | 15 | 199 |
| Heavy Chain CDR3 | 28 | 211 |
| Light Chain | 106 | 170 |
| Light Chain CDR1 | 37 | 218 |
| Light Chain CDR2 | 44 | 224 |
| Light Chain CDR3 | 50 | 229 |
| Heavy Chain Variable Region | 70 | 120 |
| Light Chain Variable Region | 94 | 157 |

TABLE 15
Sequence Identifiers for Nucleotide and
Amino acid sequences of the 178H 4B1 mAb

| | SEQ ID NO: of | SEQ ID NO: of |
|---|---|---|
| | Amino Acid | DNA |
| Sequence | Sequence | Sequence |
| Heavy Chain | 83 | 145 |
| Heavy Chain CDR1 | 8 | 194 |
| Heavy Chain CDR2 | 16 | 200 |
| Heavy Chain CDR3 | 26 | 209 |
| Light Chain | 107 | 171 |
| Light Chain CDR1 | 36 | 217 |
| Light Chain CDR2 | 44 | 224 |
| Light Chain CDR3 | 50 | 229 |
| Heavy Chain Variable Region | 71 | 121 |
| Light Chain Variable Region | 95 | 158 |

TABLE 16
Sequence Identifiers for Nucleotide and Amino
acid sequences of the Anti-Ecstasy 178K 1F4 mAb

| | SEQ ID NO: of | SEQ ID NO: of |
|---|---|---|
| | Amino Acid | DNA |
| Sequence | Sequence | Sequence |
| Heavy Chain | 85 | 147 |
| Heavy Chain CDR1 | 5 | 191 |
| Heavy Chain CDR2 | 18 | 202 |
| Heavy Chain CDR3 | 30 | 213 |
| Light Chain | 109 | 173 |
| Light Chain CDR1 | 36 | 217 |
| Light Chain CDR2 | 44 | 224 |
| Light Chain CDR3 | 50 | 229 |
| Heavy Chain Variable Region | 73 | 123 |
| Light Chain Variable Region | 97 | 160 |

TABLE 17
Sequence Identifiers for Nucleotide and Amino acid
sequences of the Anti-Ecstasy 178J 2E11 mAb

| | SEQ ID NO: of | SEQ ID NO: of |
|---|---|---|
| | Amino Acid | DNA |
| Sequence | Sequence | Sequence |
| Heavy Chain | 86 | 148 |
| Heavy Chain CDR1 | 11 | 191 |
| Heavy Chain CDR2 | 19 | 203 |
| Heavy Chain CDR3 | 31 | 214 |
| Light Chain | 110 | 174 |
| Light Chain CDR1 | 39 | 220 |
| Light Chain CDR2 | 46 | 226 |
| Light Chain CDR3 | 51 | 230 |
| Heavy Chain Variable Region | 74 | 124 |
| Light Chain Variable Region | 98 | 161 |

TABLE 18
Sequence Identifiers for Nucleotide and Amino
acid sequences of the Anti-Ecstasy 178K 1B2 mAb

| | SEQ ID NO: of | SEQ ID NO: of |
|---|---|---|
| | Amino Acid | DNA |
| Sequence | Sequence | Sequence |
| Heavy Chain | 84 | 146 |
| Heavy Chain CDR1 | 5 | 191 |
| Heavy Chain CDR2 | 17 | 201 |
| Heavy Chain CDR3 | 29 | 212 |
| Light Chain | 108 | 172 |
| Light Chain CDR1 | 38 | 219 |
| Light Chain CDR2 | 45 | 225 |
| Light Chain CDR3 | 49 | 228 |
| Heavy Chain Variable Region | 72 | 122 |
| Light Chain Variable Region | 96 | 159 |

TABLE 19
Sequence Identifiers for Nucleotide and Amino
acid sequences of the Anti-Ecstasy 178K 2B7 mAb

| | SEQ ID NO: of | SEQ ID NO: of |
|---|---|---|
| | Amino Acid | DNA |
| Sequence | Sequence | Sequence |
| Heavy Chain | 87 | 149 |
| Heavy Chain CDR1 | 5 | 191 |
| Heavy Chain CDR2 | 20 | 204 |
| Heavy Chain CDR3 | 29 | 212 |
| Light Chain | 111 | 175 |
| Light Chain CDR1 | 40 | 221 |
| Light Chain CDR2 | 47 | 227 |
| Light Chain CDR3 | 52 | 231 |
| Heavy Chain Variable Region | 75 | 125 |
| Light Chain Variable Region | 99 | 162 |

TABLE 20
Sequence Identifiers for Nucleotide and Amino
acid sequences of the Anti-Ecstasy 178K 3C8 mAb

| | SEQ ID NO: of | SEQ ID NO: of |
|---|---|---|
| | Amino Acid | DNA |
| Sequence | Sequence | Sequence |
| Heavy Chain | 88 | 150 |
| Heavy Chain CDR1 | 9 | 195 |
| Heavy Chain CDR2 | 21 | 205 |
| Heavy Chain CDR3 | 32 | 215 |
| Light Chain | 112 | 176 |
| Light Chain CDR1 | 41 | 222 |
| Light Chain CDR2 | 44 | 224 |
| Light Chain CDR3 | 50 | 229 |
| Heavy Chain Variable Region | 76 | 126 |
| Light Chain Variable Region | 100 | 163 |

TABLE 21
Sequence Identifiers for Nucleotide and Amino acid
sequences of the Anti-Ecstasy 178K 4E11 mAb

| | SEQ ID NO: of | SEQ ID NO: of |
|---|---|---|
| | Amino Acid | DNA |
| Sequence | Sequence | Sequence |
| Heavy Chain | 89 | 151 |
| Heavy Chain CDR1 | 5 | 191 |
| Heavy Chain CDR2 | 22 | 206 |
| Heavy Chain CDR3 | 29 | 212 |
| Light Chain | 113 | 177 |
| Light Chain CDR1 | 38 | 219 |
| Light Chain CDR2 | 47 | 227 |
| Light Chain CDR3 | 49 | 228 |
| Heavy Chain Variable Region | 77 | 127 |
| Light Chain Variable Region | 101 | 164 |

TABLE 22
Sequence Identifiers for Nucleotide and Amino acid
sequences of the Anti-Ecstasy 178K 5B11 mAb

| | SEQ ID NO: of | SEQ ID NO: of |
|---|---|---|
| | Amino Acid | DNA |
| Sequence | Sequence | Sequence |
| Heavy Chain | 90 | 152 |
| Heavy Chain CDR1 | 10 | 196 |
| Heavy Chain CDR2 | 23 | 207 |
| Heavy Chain CDR3 | 29 | 212 |
| Light Chain | 114 | 178 |
| Light Chain CDR1 | 42 | 223 |
| Light Chain CDR2 | 44 | 224 |
| Light Chain CDR3 | 49 | 228 |
| Heavy Chain Variable Region | 78 | 128 |
| Light Chain Variable Region | 102 | 165 |

### Example 19: Binding Affinities of Anti-Ecstasy Compound-Specific Monoclonal Antibodies

The binding affinities of the anti-ecstasy compound-specific mAbs for MDMA, MDA, amphetamine, and methamphetamine was performed on a Biacore™ T200 instrument (GE Healthcare) using a Series S CM5 sensor chip, buffers, amine coupling kit and regeneration solutions from Cytiva. The avidity effect associated with bivalency of the intact antibodies was avoided and the 1:1 binding model requirement was satisfied by conducting the experiment with the Fab fragments of the anti-ecstasy mAbs. The Fab fragments were prepared by papain digestion of the mAbs in the presence of 10 mM L-cysteine or 12.5 mM-mercaptoethanol in PBS, pH 7.0 containing 2 mM Na₂EDTA at an enzyme/mAb ratio of 1:200. After incubation at 37° C. for 1-18 h papain hydrolysis was stopped with 20 mM iodacetamide. The Fab fragments were purified in flow-through mode by capturing the digested Fc fragments with recombinant Protein A Sepharose Fast Flow (GE Life Science) and/or gel filtration on HiLoad 16/600 Superdex 200 μg column (Cytiva). Homogeneity and integrity of the Fab fragments was determined by SDS-PAGE under reduced and non-reduced conditions (data not shown).

An affinity in solution protocol was used to determine the affinities of the interactions of the anti-ecstasy compound Fab fragments with low molecular weight compounds, such as MDMA, MDA, amphetamine, and methamphetamine. In this format, an immobilized antigen (E1-OVA (55)) and a low molecular weight antigen in solution (inhibitor) compete for binding sites on the common Fab fragment. The initial binding rate of the Fab fragment to the immobilized antigen is constant and directly proportional to the Fab fragment concentration in solution under mass transport limited conditions. A full description of this protocol can be found in Adamczyk, et al., Methods, 2000, 20; p. 319-328, which is incorporated herein by reference. The concentration of the free Fab fragment available for binding to E1-OVA (55) (not bound to the inhibitor) is calculated from a calibration curve prepared separately by running a known concentration of the Fab fragment over the same sensor surface under mass transport limited conditions. The equilibrium dissociation constant (K_{D}) for the inhibitor/Fab fragment interaction is calculated using the solution 1:1 affinity model according to the equation:

$${Fab}_{free} = \frac{( Fab - Inh - {K}_{D} )}{2} \pm \sqrt{\frac{{( Inh + Fab + {K}_{D} )}^{2}}{4} - Inh \cdot Fab}$$

where “Fabfree” is the concentration of free Fab fragment in solution, “Fab” is the total concentration of the Fab fragment, and “Inh” is the total concentration of the low molecular weight inhibitor.

To measure the concentration of free anti-Ecstasy Fab fragment in solution a high-density E1-OVA (55) biosensor surface (~1,300 RU) was prepared as follows. A new Series S CM5 sensor chip was washed with HBS-N buffer and activated by a 7 min injection of 200 mM EDC and 50 mM SuOH at a flow rate of 10 μL/min. E1-OVA (55) (20 μg/mL in 10 mM sodium acetate, pH 4.0) was immobilized on the EDC/SuOH-activated chip surface during the 7 min injection, followed by a 7 min injection of 1 M ethanolamine HCl (pH 8.5) to block the excess of active ester groups. The reference surface was prepared in the same manner using a conjugate of a non-ecstasy compound and ovalbumin.

Affinity assays were performed in HBS-P+ running buffer at 25° C. To generate calibration curve for solution affinity analysis, seven concentrations of the anti-ecstasy compound Fab fragment (two-fold dilution from 2 to 128 nM) were injected over the surface of the chip for 7 min at 2 μL/min followed by a 1 min of dissociation and 1 min surface regeneration with 10 mM glycine HCl, pH 2.0. It was confirmed that the anti-ecstasy compound Fab fragment binding to the immobilized E1-OVA (55) was mass transport limited by varying the flow rate on separate injections (data not shown). The response from reference surface was subtracted from the E1-OVA (55) sensograms to remove the bulk effect. An initial binding rate for each Fab fragment concentration tested was determined by measuring the sensogram slope 15 sec after Fab fragment injection. The calibration curve was prepared by four-parameter fit of a nonlinear regression plot of the initial binding rate vs Fab fragment concentration.

For inhibition analysis, 64 nM of the anti-ecstasy compound Fab fragment was equilibrated with varying concentrations of inhibitors (MDA, MDMA, amphetamine, or methamphetamine) in running buffer for 2 h at 25° C. The inhibitor/Fab fragment samples were then run over the E1-OVA (55)-immobilized chip surface and a sensogram slope was recorded 15 sec after sample injection. Flow parameters and binding conditions were the same as that used for calibration curve generation. Affinity in solution assay data were collected over 2-2,000 nM MDA or MDMA and 16-16,000 μM amphetamine or methamphetamine concentrations. The amount of free Fab fragment in solution was determined from the calibration curve and plotted against inhibitor concentrations using the BIAevaluation software version 3.2.1. The K_{D} for the inhibitor/Fab fragment binding was calculated using the solution 1:1 affinity model described above.

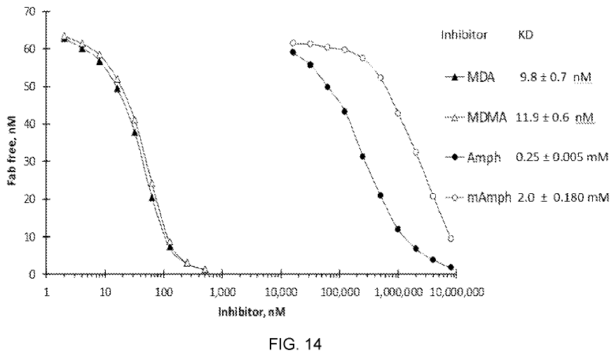

An example of an affinity in solution experiment to determine the affinity between MDMA and a 178F 4H5 mAb Fab fragment is shown in FIG. 13. Increasing concentrations of the 178F 4H5 mAb Fab fragment were injected over E1-OVA (55) and the initial slope of each response was used to generate a calibration curve (FIG. 13A). MDMA in varying concentrations was incubated with a constant concentration (64 mM) of the 178F 4H5 mAb Fab fragment and allowed to reach equilibrium. The MDMA/Fab fragment samples were injected over the E1-OVA (55) surface and the amount of Fab fragment in solution that was not bound to MDMA (free Fab fragment) was determined from the calibration curve. The amount of free Fab fragment was plotted against MDMA concentration using the BIAevaluation software version 3.2.1 (FIG. 13B). The K_{D} for the interaction of MDMA with 178F 4H5 mAb Fab fragment was determined to be 11.9±0.6 nM. The K_{D} for the MDA, amphetamine, and methamphetamine interactions was similarly calculated and presented in Table 23. Comparison of 178F 4H5 mAb Fab fragment affinities for interaction with MDMA, MDA, Amph and mAmph is presented in FIG. 14. The data show that mAbs generated in response to the E1-KLH (56) immunogen are highly specific to ecstasy class compounds (e.g., MDA and MDMA). That is, they bind both MDMA and its MDA metabolite with nanomolar affinities, but their affinities to amphetamine and methamphetamine are very low (about 0.2-2.7 mM). Due to the minimal differences in affinities to MDMA and MDA, these mAbs are capable of binding MDA and MDMA with similar efficiency (80-100% recovery) and also distinguishing ecstasy class compounds MDMA and MDA from structure-related, non-ecstasy class compounds (e.g., amphetamine or methamphetamine).

TABLE 23

| | K_{D} (nM) | | K_{D} (mM) | |
|---|---|---|---|---|
| mAb | MDMA | MDA | Amph | mAmph |
| 178 4C12 | 3.9 ± 1.0 | 3.7 ± 1.3 | 0.33 ± 0.01 | 1.9 ± 0.01 |
| 178F 4H5 | 11.9 ± 0.6 | 9.8 ± 0.7 | 0.25 ± 0.01 | 2.0 ± 0.02 |
| 178H 2A7 | 11.1 ± 0.4 | 11.3 ± 0.6 | 0.53 ± 0.01 | 2.7 ± 0.02 |
| 178H 4B1 | 11.4 ± 1.0 | 6.6 ± 0.5 | 0.28 ± 0.02 | 2.1 ± 0.01 |
| 178K 2B7 | 11.6 ± 1.0 | 6.6 ± 0.3 | 0.28 ± 0.01 | 2.5 ± 0.02 |
| 178K 3C8 | 13.3 ± 0.4 | 5.8 ± 0.6 | 0.29 ± 0.01 | 2.5 ± 0.01 |

### Example 20: Prototype EMIT Ecstasy Assay Using MDMA-G6PDH Conjugate

Anti-ecstasy antibodies and E1-G6PDH (58) were evaluated in an EMIT assay format. The EMIT format assay is a homogenous enzyme immunoassay technique used for the analysis of specific compounds in human urine. The assay is based on competition for antibody binding sites between a drug in a sample and the drug labelled with a marker, such as G6PDH. Enzyme activity decreases upon binding to the antibody, so the drug concentration in the sample can be measured in terms of enzyme activity. When the marker is G6PDH, the enzyme converts nicotinamide adenine dinucleotide (NAD) to NADH in the presence of glucose-6-phosphate (G6P), resulting in an absorbance change that is measured spectrophotometrically. Endogenous G6PDH does not interfere with the assay because the coenzyme NAD functions only with the bacterial enzyme (from Leuconostoc mesenteroides) employed in the assay.

Anti-ecstasy antibodies were spiked to EMIT format antibody diluent (EMIT diluent SMN 10872252-R¹) at a loading point between 3.5 and 7.5 g/mL. The EMIT antibody diluent contains BSA, G6P, NAD, preservatives, and stabilizers. E1-G6PDH (58) was added to the enzyme conjugate diluent (EMIT diluent SMN 10872252-R²) and adjusted to a maximum rate between 620-720 mA/min to produce the enzyme conjugate reagent. The conjugate diluent contains MDA labeled with bacterial recombinant G6PDH, HEPES buffer, BSA, preservatives, and stabilizers. MDMA was spiked into a urine pool at concentrations of 0, 150, 300, 500, and 1000 ng/mL for calibrators. The assay calibration curves were generated using a VIVA-ER System (Siemens Healthineers). The assay reaction was monitored at 340 nm using an incubation time of 50 sec and a read time of 106 sec. This assay can demonstrate the affinity and specificity of the antibodies described herein.

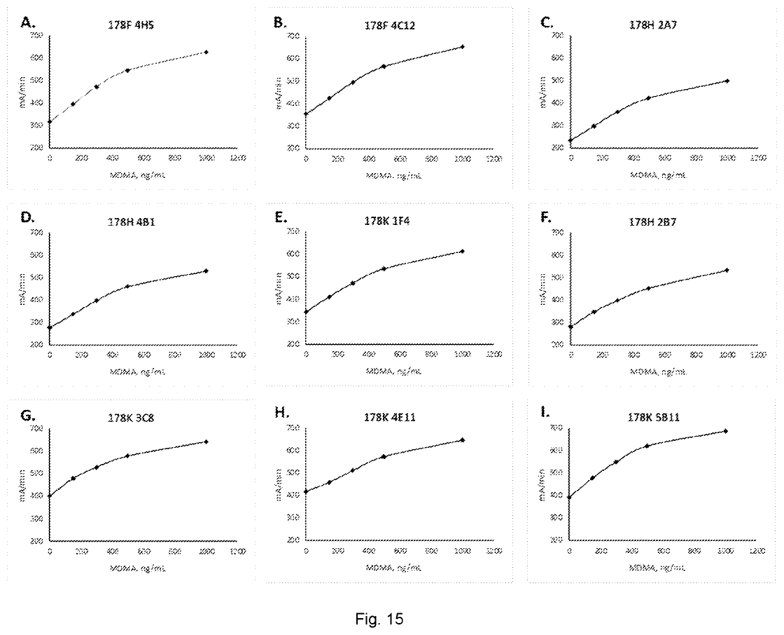

FIGS. 15A-K show the change in absorbance of the monoclonal antibody clones 178F 4H5 (A), 178F 4C12 (B), 178H 2A7 (C), 178H 4B1 (D), 178K IF4 (E), 178K 2B7 (F), 178K 3C8 (G), 178K 4E11 (H), and 178K 5B11 (I), respectively, in an EMIT ecstasy assay using the MDA-G6PDH conjugate described above.

A negative human urine sample was spiked with MDA and tested using anti-ecstasy mAbs and a G6PDH-MDA conjugate. The recovery was calculated using the MDMA calibration curve on the VIVA-ER System (Siemens Healthineers).

Following incubation, G6P and NAD⁺ were added to the sample. MDA recovery was calculated based on the absorbance at 340 nm using an incubation time of 50 sec and a read time of 106 sec. Table 24 shows the percent recovery of MDA, which meets the SAMHSA requirement of a ≥80% at cutoff the level of 500 ng/ml. Amphetamine (“Amph”) and methamphetamine (“mAmph”) were spiked into another negative urine pool at a concentration of 500 ng/mL. These concentrations produced a response which was equivalent to the 500 ng/mL cutoff. These results demonstrate that EMIT assaying using the MDA conjugated to G6PDH (an embodiment of Formula (I)) and antibodies raised against the MDA conjugated to KLH as the immunogenic carrier (another embodiment of Formula (I)) recover over 80% of the MDA and MDMA in a sample, thereby meeting the new SAMHSA guidelines. Furthermore, the antibodies have low cross-reactivity with amphetamine and methamphetamine.

TABLE 24

| | Compound 58 | | | | | | | | |
|---|---|---|---|---|---|---|---|---|---|
| Conjugate | 178F | 178F | 178H | 178H | 178K | 178K | 178K | 178K | 178K |
| Antibody | 4H5 | 4C12 | 2A7 | 4B1 | 1F4 | 2B7 | 3C8 | 4E11 | 5B11 |
| Curve Size | 239 | 246 | 274 | 264 | 263 | 235 | 257 | 221 | 299 |
| (mA/min) | | | | | | | | | |
| MDA (500 | 480 | 428 | 399 | 460 | 591 | 426 | 640 | 501 | 507 |
| ng/mL) | | | | | | | | | |
| MDA | 96% | 86% | 80% | 92% | 118% | 85% | 128% | 100% | 101% |
| Recovery | | | | | | | | | |

Concentrations (μg/mL) of Structurally Related
Compounds Equivalent to 500 ng/mL MDMA Cutoffs

| | | | | | | | | | |
|---|---|---|---|---|---|---|---|---|---|
| (+/−) Amph | 30 | 40-45 | 40-45 | 40 | 30 | 40-45 | 30 | 40-45 | 25-30 |
| (+/−) | 30-40 | 40-50 | 30-40 | 40-50 | 30 | 50-60 | 50 | 50-60 | 30-50 |
| mAmph | | | | | | | | | |

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A compound corresponding in structure to a Formula (I): 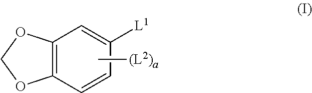
wherein
L¹ is 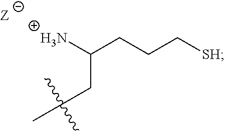 [SMILES: *CC([NH3+])CCCS]
a is zero or 1;
L² is —(CH₂)ᵢ—(X⁵)—(Y⁵)ⱼ;
X⁵ is —SH, —NH₂, —COOH, or —S(CH₂)(CO)NH—,
Y⁵ is an immunogenic carrier or a label;
I is 1 to 12;
j is zero or 1; and
Z is Cl, (CF₃)CO₂, F, or Br.

2.-13. (canceled)

14. The compound of claim 1 corresponding in structure to Formula (Id): 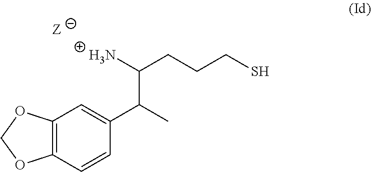
wherein Z is Cl, (CF₃)CO₂, F, or Br.

15. (canceled)

16. A compound corresponding in structure to a Formula (I): 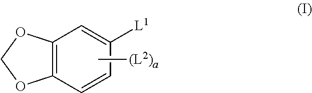
wherein
L¹ is 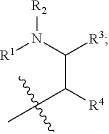
R¹ is hydrogen, an alkyl, or —CO(CF₃);
R² is hydrogen, an alkyl, or —(CH₂)_{b}—(X¹)—(Y¹)_{c};
X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or 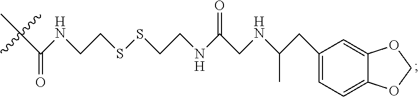 [SMILES: *C(=O)NCCSSCCNC(=O)CNC(C)Cc1ccc2c(c1)OCO2]
Y¹ is an immunogenic carrier or a label;
b is 1 to 10;
c is zero or 1;
R³ is hydrogen, alkyl, or —(CH₂)_{d}—(X²)—(Y²)ₑ;
X² is —SH, —NH₂, —COOH, —CONH—, —S(CH₂)(CO)NH—, —CO(NH)(C₂H₄)S(CH₂)(CO)NH—, or 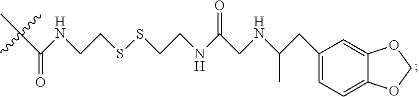 [SMILES: *C(=O)NCCSSCCNC(=O)CNC(C)Cc1ccc2c(c1)OCO2]
Y² is an immunogenic carrier or a label;
d is 2 to 12;
e is zero or 1;
R⁴ is hydrogen or —(CH₂)_{f}(X³)—(Y³)_{g};
X³ is —SH, —NH₂, —COOH, —CONH—, —S(CH₂)(CO)NH—, —CO(NH)(C₂H₄)S(CH₂)(CO)NH—, or 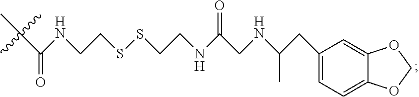 [SMILES: *C(=O)NCCSSCCNC(=O)CNC(C)Cc1ccc2c(c1)OCO2]
Y³ is an immunogenic carrier or a label;
f is 1 to 12;
g is zero or 1;
a is zero or 1;
L² is —(CH₂)ᵢ—(X⁵)(Y⁵)ⱼ;
X⁵ is —SH, —NH₂, —COOH, or —S(CH₂)(CO)NH—,
Y⁵ is an immunogenic carrier or a label;
i is 1 to 12; and
j is zero or 1;
wherein when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ is hydrogen;
wherein when a is zero, R¹ is hydrogen or methyl, R² is —(CH₂)_{b}—(X¹)—(Y¹)_{c}, X¹ is —CONH—, c is 1, Y¹ is an immunogenic carrier or a label, R³ is methyl, R⁴ is hydrogen, then b is 5-10;
wherein when a is zero, R² is —(CH₂)_{b}—(X¹)—(Y¹)_{c}, b is 3 or 4, X¹ is —COOH, c is zero, R³ is methyl, R⁴ is hydrogen, then R¹ is hydrogen;
wherein when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b is 5-10; and
wherein at least one of R¹, R², and R³ is neither hydrogen nor an alkyl.

17.-19. (canceled)

20. The compound of claim 16, corresponding in structure to Formula (Ia): 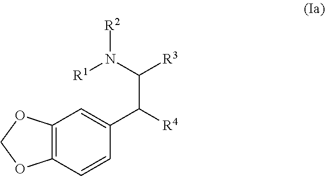
wherein
R¹ is hydrogen;
R² is hydrogen or C₁₋₆ alkyl;
R³ is —(CH₂)_{d}—(X²)—(Y²)_{c};
X² is —S(CH₂)(CO)NH—,
Y² is an immunogenic carrier or a label;
d is 2 to 6;
e is 1;
R⁴ is hydrogen.

21.-27. (canceled)

28. The compound of claim 20, wherein the compound corresponds in structure to: 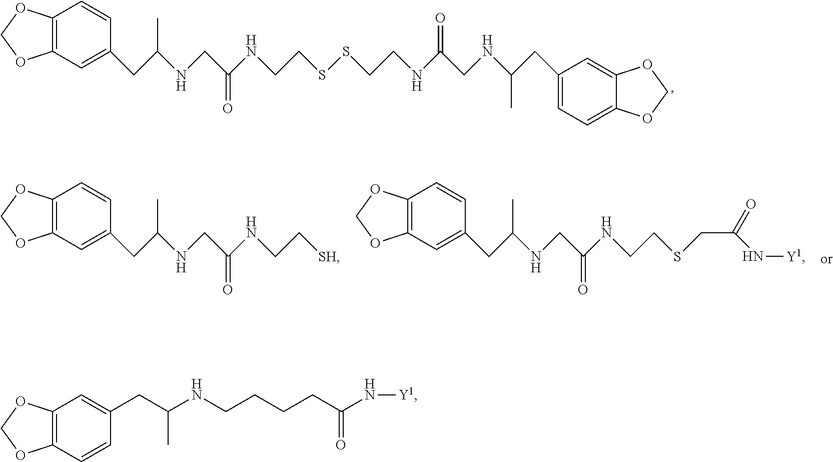
wherein Y¹ is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

29.-32. (canceled)

33. The compound of claim 20, wherein the compound corresponds in structure to: 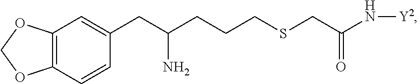
wherein Y² is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

34. The compound of claim 16 corresponding in structure to Formula (Ib): 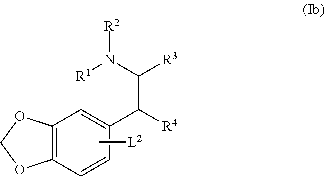
wherein
R¹ is hydrogen, an alkyl, or —CO(CF₃);
R² is hydrogen, an alkyl, or —(CH₂)_{b}—(X¹)—(Y¹)_{c};
X¹ is —SH, —NH₂, —COOH, —CONH—, —S(CH₂)(CO)NH—, —CO(NH)(C₂H₄)S(CH₂)(CO)NH—, or 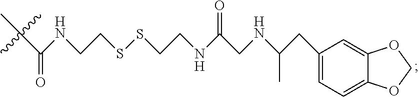 [SMILES: *C(=O)NCCSSCCNC(=O)CNC(C)Cc1ccc2c(c1)OCO2]
Y¹ is an immunogenic carrier or a label;
b is 1 to 10;
c is zero or 1;
R³ is hydrogen, alkyl, or —(CH₂)_{d}—(X²)—(Y²)_{c};
X² is —SH, —NH₂, —COOH, —CONH—, —S(CH₂)(CO)NH—, —CO(NH)(C₂H₄)S(CH₂)(CO)NH—, or 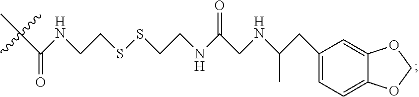 [SMILES: *C(=O)NCCSSCCNC(=O)CNC(C)Cc1ccc2c(c1)OCO2]
Y² is an immunogenic carrier or a label;
d is 2 to 12;
e is zero or 1;
R⁴ is hydrogen or —(CH₂)_{f}(X³)—(Y³)_{g};
X³ is —SH, —NH₂, —COOH, —CONH—, —S(CH₂)(CO)NH—, —CO(NH)(C₂H₄)S(CH₂)(CO)NH—, or 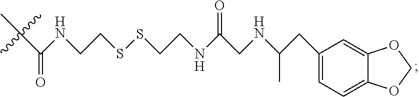 [SMILES: *C(=O)NCCSSCCNC(=O)CNC(C)Cc1ccc2c(c1)OCO2]
Y³ is an immunogenic carrier or a label;
f is 1 to 12; and
g is zero or 1;
L² is —(CH₂)ᵢ—(X⁵)(Y⁵)ⱼ;
X⁵ is —SH, —NH₂, —COOH, or —S(CH₂)(CO)NH—,
Y⁵ is an immunogenic carrier or a label;
i is 1 to 12; and
j is zero or 1.

35.-39. (canceled)

40. The compound of claim 34, wherein the compound corresponds in structure to: 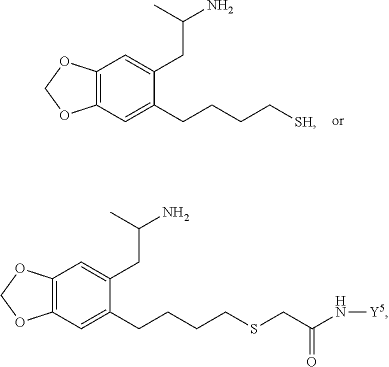
wherein Y⁵ is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

41. The compound of claim 1, wherein the immunogenic carrier is selected from the group consisting of a protein, a polypeptide, and a polysaccharide, wherein the protein is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

42. The compound of claim 16, wherein the immunogenic carrier is selected from the group consisting of a protein, a polypeptide, and a polysaccharide, wherein the protein is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

43. An antibody raised against a compound according to claim 41, wherein the antibody is raised against the compound corresponding in structure to 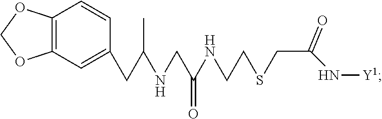
wherein Y¹ is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

44. An antibody raised against a compound according to claim 42, wherein the antibody is raised against the compound corresponding in structure to 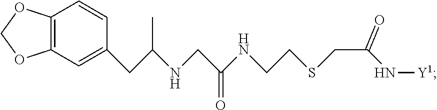
wherein Y¹ is selected from the group consisting of keyhole limpet hemocyanin (KLH), bovine serum albumin (BSA), bovine thyroglobulin (BTG), egg ovalbumin (OVA), bovine gamma globulin (BGG), and glucose-6-phosphate dehydrogenase (G6PDH).

45. The antibody of claim 43, wherein the antibody binds to an ecstasy-class compound selected from the group consisting of 3,4-methylenedioxymethamphetamine (MDMA) and 3,4-methylenedioxyamphetamine (MDA).

46. (canceled)

47. The antibody of claim 43, wherein the antibody recovers at least 80% of MDA in a sample from an immunoassay.

48. The antibody of claim 43, wherein the antibody has a K_{D} for MDA of about 10⁻⁵ M to about 10⁻⁹M and/or a K_{D} for MDMA of about 10⁻⁵M to about 10⁻ M.

49. (canceled)

50. (canceled)

51. The antibody of claim 43, wherein the antibody comprises:
a variable heavy chain comprising:
a first heavy chain complementarity determining region (HC-CDR 1) as set forth in SEQ ID NO:4;
a second heavy chain complementarity determining region (HC-CDR 2) as set forth in SEQ ID NO:12; and
a third heavy chain complementarity determining region (HC-CDR 3) as set forth in SEQ ID NO:24; and
a variable light chain comprising:
a first light chain complementarity determining region (LC-CDR 1) as set forth in SEQ ID NO:34;
a second light chain complementarity determining region (LC-CDR 2) as set forth in SEQ ID NO:43; and
a third light chain complementarity determining region (LC-CDR 2) as set forth in SEQ ID NO:48.

52. The antibody of claim 43, wherein
the HC-CDR 1 comprises an amino acid sequence having at least 80% sequence identity with the sequence selected from the group consisting of SEQ ID NO:5, SEQ ID NO:6, SEQ ID NO:7, SEQ ID NO:8, SEQ ID NO:9, and SEQ ID NO:10;
the HC-CDR 2 comprises an amino acid sequence having at least 80% sequence identity with the sequence selected from the group consisting of SEQ ID NO:13, SEQ ID NO:14, SEQ ID NO:15, SEQ ID NO:16, SEQ ID NO:17, SEQ ID NO:18, SEQ ID NO:20, SEQ ID NO:21, SEQ ID NO:22, and SEQ ID NO:23; and
the HC-CDR 3 comprises an amino acid sequence having at least 80% sequence identity with the sequence selected from the group consisting of SEQ ID NO:26, SEQ ID NO:27, SEQ ID NO:28, SEQ ID NO:29, SEQ ID NO:30, and SEQ ID NO:32; and/or
the LC-CDR 1 comprises an amino acid sequence having at least 80% sequence identity with the sequence selected from the group consisting of SEQ ID NO:35, SEQ ID NO:36, SEQ ID NO:37, SEQ ID NO:38, SEQ ID NO:40, SEQ ID NO:41, and SEQ ID NO:42;
the LC-CDR 2 comprises an amino acid sequence having at least 80% sequence identity with the sequence selected from the group consisting of SEQ ID NO:44, SEQ ID NO:45, and SEQ ID NO:47; and
the LC-CDR 3 comprises an amino acid sequence having at least 80% sequence identity with the sequence selected from the group consisting of SEQ ID NO:49, SEQ ID NO:50, and SEQ ID NO:52.

53. (canceled)

54. The antibody of claim 43, wherein the variable heavy chain has an amino acid sequence having at least 80% sequence identity with the sequence selected from the group consisting of SEQ ID NO:68, SEQ ID NO:69, SEQ ID NO:70, SEQ ID NO:71, SEQ ID NO:72, SEQ ID NO:73, SEQ ID NO:75, SEQ ID NO:76, SEQ ID NO:77, and SEQ ID NO:78; and/or wherein the variable light chain has an amino acid sequence having at least 80% sequence identity with the sequence selected from the group consisting of SEQ ID NO:92, SEQ ID NO:93, SEQ ID NO:94, SEQ ID NO:95, SEQ ID NO:96, SEQ ID NO:97, SEQ ID NO:99, SEQ ID NO:100, SEQ ID NO:101, and SEQ ID NO:102.

55. (canceled)

56. The antibody of claim 43, wherein the variable heavy chain is covalently attached to a heavy chain constant region having an amino acid sequence as set forth in SEQ ID NO:53 to form a full heavy chain and/or the variable light chain is covalently attached to a light chain constant region having an amino acid sequence having at least 80% sequence identity with SEQ ID NO:66 to form a full light chain.

57. (canceled)

58. The antibody of claim 43, wherein the full heavy chain comprises an amino acid sequence having at least 80% sequence identity with the sequence selected from the group consisting of SEQ ID NO:80, SEQ ID NO:81, SEQ ID NO:82, SEQ ID NO:83, SEQ ID NO:84, SEQ ID NO:85, SEQ ID NO:87, SEQ ID NO:88, SEQ ID NO:89, and SEQ ID NO:90; and/or the full light chain comprises an amino acid sequence having at least 80% sequence identity with the sequence selected from the group consisting of SEQ ID NO:104, SEQ ID NO:105, SEQ ID NO:106 SEQ ID NO:107, SEQ ID NO:108, SEQ ID NO:109, SEQ ID NO:111, SEQ ID NO:112, SEQ ID NO:113, and SEQ ID NO:114.

59.-64. (canceled)

65. The antibody of claim 43, wherein the isolated monoclonal antibody consists of a pair of a full heavy chain and a full light chain, wherein the pair is selected from the group consisting of:
SEQ ID NOs:80 and 104;
SEQ ID NOs:81 and 105;
SEQ ID NOs:82 and 106;
SEQ ID NOs:83 and 107;
SEQ ID NOs:84 and 108;
SEQ ID NOs:85 and 109;
SEQ ID NOs:87 and 111;
SEQ ID NOs:88 and 112;
SEQ ID NOs:89 and 113; and
SEQ ID NOs:90 and 114.

66. A polynucleotide comprising a DNA sequence encoding an antibody of claim 43.

67. The polynucleotide of claim 66, wherein the DNA sequence encoding the variable heavy chain has a sequence having at least 80% sequence identity with the sequence selected from the group consisting of SEQ ID NO:118, SEQ ID NO:119, SEQ ID NO:120 SEQ ID NO:121, SEQ ID NO:122, SEQ ID NO:123, SEQ ID NO:125, SEQ ID NO:126, SEQ ID NO:127, and SEQ ID NO:128; and/or the DNA sequence encoding the variable light chain has a sequence having at least 80% sequence identity with the sequence selected from the group consisting of SEQ ID NO:155, SEQ ID NO:156, SEQ ID NO:157, SEQ ID NO:158, SEQ ID NO:159, SEQ ID NO:160, SEQ ID NO:162, SEQ ID NO:163, SEQ ID NO:164, and SEQ ID NO:165.

68. (canceled)

69. (canceled)

70. The polynucleotide of claim 66, wherein the DNA sequence encoding the full heavy chain has a sequence having at least 80% sequence identity with the sequence selected from the group consisting of SEQ ID NO:142, SEQ ID NO:143, SEQ ID NO:144, SEQ ID NO:145, SEQ ID NO:146, SEQ ID NO:147, SEQ ID NO:149, SEQ ID NO:150, SEQ ID NO:151, and SEQ ID NO:152; and/or the DNA sequence encoding the variable light chain has a sequence having at least 80% sequence identity with the sequence selected from the group consisting of SEQ ID NO:168, SEQ ID NO:169, SEQ ID NO:170, SEQ ID NO:171, SEQ ID NO:172, SEQ ID NO:173, SEQ ID NO:175, SEQ ID NO:176, SEQ ID NO:177, and SEQ ID NO:178.

71.-74. (canceled)

75. A kit comprising in packaged combination:
(i) the antibody of claim 43; and
(ii) a conjugate of an enzyme and an MDA analog and/or a conjugate of an enzyme and an MDMA analog, wherein the conjugate corresponds in structure to Formula (I): 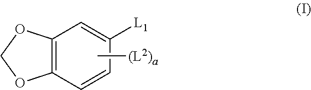
wherein
L¹ is 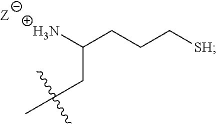
a is zero or 1;
L² is —(CH₂)ⱼ—(X⁵)—(Y⁵)ⱼ;
X⁵ is —SH, —NH₂, —COOH, or —S(CH₂)(CO)NH—,
Y⁵ is an immunogenic carrier or a label;
i is 1 to 12;
j is zero or 1; and
Z is Cl, (CF₃)CO₂, F, or Br.

76. A kit comprising in packaged combination:
(i) the antibody of claim 44; and
(ii) a conjugate of an enzyme and an MDA analog and/or a conjugate of an enzyme and an MDMA analog, wherein the conjugate corresponds in structure to Formula (I): 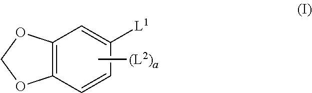
wherein
L¹ is 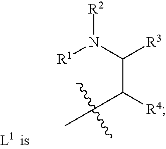
R¹ is hydrogen, an alkyl, or —CO(CF₃);
R² is hydrogen, an alkyl, or —(CH₂)_{b}—(X¹)—(Y¹)_{c};
X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or 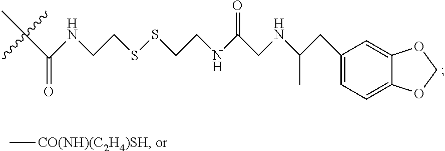
Y¹ is an immunogenic carrier or a label;
b is 1 to 10;
c is zero or 1;
R³ is hydrogen, alkyl, or —(CH₂)_{d}—(X²)—(Y²)ₑ;
X² is —SH, —NH₂, —COOH, —CONH—, —S(CH₂)(CO)NH—, —CO(NH)(C₂H₄)S(CH₂)(CO)NH—, or 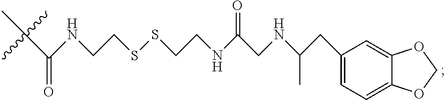 [SMILES: *C(=O)NCCSSCCNC(=O)CNC(C)Cc1ccc2c(c1)OCO2]
Y² is an immunogenic carrier or a label;
d is 2 to 12;
e is zero or 1;
R⁴ is hydrogen or —(CH₂)_{f}(X³)—(Y³)_{g};
X³ is —SH, —NH₂, —COOH, —CONH—, —S(CH₂)(CO)NH—, —CO(NH)(C₂H₄)S(CH₂)(CO)NH—, or 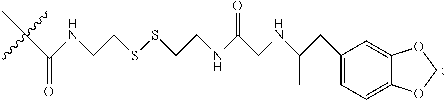 [SMILES: *C(=O)NCCSSCCNC(=O)CNC(C)Cc1ccc2c(c1)OCO2]
Y³ is an immunogenic carrier or a label;
f is 1 to 12;
g is zero or 1;
a is zero or 1;
L² is —(CH₂)ₙ—(X⁵)—(Y⁵)ⱼ;
X⁵ is —SH, —NH₂, —COOH, or —S(CH₂)(CO)NH—,
Y⁵ is an immunogenic carrier or a label;
i is 1 to 12; and
j is zero or 1;
wherein when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ is hydrogen;
wherein when a is zero, R¹ is hydrogen or methyl, R² is —(CH₂)_{b}—(X¹)—(Y¹)_{c}, X¹ is —CONH—, c is 1, Y¹ is an immunogenic carrier or a label, R³ is methyl, R⁴ is hydrogen, then b is 5-10;
wherein when a is zero, R² is —(CH₂)_{b}—(X¹)—(Y¹)_{c}, b is 3 or 4, X¹ is —COOH, c is zero, R³ is methyl, R⁴ is hydrogen, then R¹ is hydrogen;
wherein when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b is 5-10; and
wherein at least one of R¹, R², and R³ is neither hydrogen nor an alkyl.

77. (canceled)

78. A method for determining the presence of MDA and/or MDMA in a sample suspected of containing MDA and/or MDMA, the method comprising:
providing the sample, a conjugate of an enzyme and an MDA analog and/or a conjugate of an enzyme and an MDMA analog, and the antibody of claim 43 to a medium; and
examining the medium for the presence of a complex comprising the MDA and/or MDMA and the antibody,
wherein the conjugate corresponds in structure to a Formula (I): 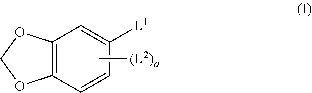
wherein
L¹ is 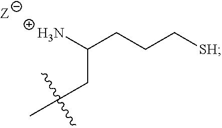 [SMILES: *CC([NH3+])CCCS]
a is zero or 1;
L² is —(CH₂)ᵢ—(X⁵)—(Y⁵)ⱼ;
X⁵ is —SH, —NH₂, —COOH, or —S(CH₂)(CO)NH—,
Y⁵ is an immunogenic carrier or a label;
i is 1 to 12;
j is zero or 1; and
Z is Cl, (CF₃)CO₂, E, or Br.

79. The method of claim 78, further comprising the steps of:
incubating the sample, conjugate, and antibody for a time sufficient for the antibody to bind to the MDA and/or MDMA in the sample;
adding a substrate for the enzyme to the sample; and
measuring the activity of the enzyme;
wherein the presence of the complex comprising the MDA and/or MDMA and the antibody is proportional to the activity of the enzyme.

80. A method for determining the presence of MDA and/or MDMA in a sample suspected of containing MDA and/or MDMA, the method comprising:
providing the sample, a conjugate of an enzyme and an MDA analog and/or a conjugate of an enzyme and an MDMA analog, and the antibody of claim 44 to a medium; and
examining said medium for the presence of a complex comprising the MDA and/or MDMA and the antibody,
wherein the conjugate corresponds in structure to a Formula (I): 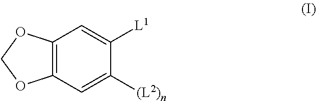
wherein
L¹ is 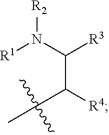
R¹ is hydrogen, an alkyl, or —CO(CF₃);
R² is hydrogen, an alkyl, or —(CH₂)_{b}—(X¹)—(Y¹)_{c};
X¹ is —SH, —NH₂, —COOH, —CONH—, —S(CH₂)(CO)NH—, —CO(NH)(C₂H₄)S(CH₂)(CO)NH—, —CO(NH)(C₂H₄)SH, or 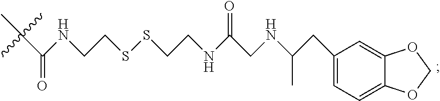 [SMILES: *C(=O)NCCSSCCNC(=O)CNC(C)Cc1ccc2c(c1)OCO2]
Y¹ is an immunogenic carrier or a label;
b is 1 to 10;
c is zero or 1;
R³ is hydrogen, alkyl, or —(CH₂)_{d}—(X²)—(Y²)_{c};
X² is —SH, —NH₂, —COOH, —CONH—, —S(CH₂)(CO)NH—, —CO(NH)(C₂H₄)S(CH₂)(CO)NH—, or 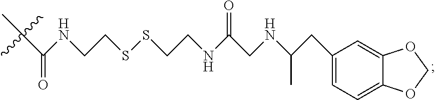 [SMILES: *C(=O)NCCSSCCNC(=O)CNC(C)Cc1ccc2c(c1)OCO2]
Y² is an immunogenic carrier or a label;
d is 2 to 12;
e is zero or 1;
R⁴ is hydrogen or —(CH₂)_{f}(X³)—(Y³)_{g};
X³ is —SH, —NH₂, —COOH, —CONH—, —S(CH₂)(CO)NH—, —CO(NH)(C₂H₄)S(CH₂)(CO)NH—, or 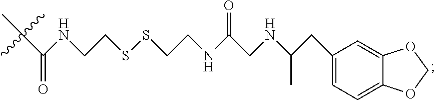 [SMILES: *C(=O)NCCSSCCNC(=O)CNC(C)Cc1ccc2c(c1)OCO2]
Y³ is an immunogenic carrier or a label;
f is 1 to 12;
g is zero or 1;
a is zero or 1;
L² is —(CH₂); (X⁵)ᵢ—(Y⁵)ⱼ;
X⁵ is —SH, —NH₂, —COOH, or —S(CH₂)(CO)NH—,
Y⁵ is an immunogenic carrier or a label;
i is 1 to 12; and
j is zero or 1;
wherein when a is zero, R² is —(CH₂)_{b}—(X¹)—(Y¹)_{c}, b is 1 or 4, X¹ is —CONH—, c is 1, Y¹ is an immunogenic carrier or a label, R³ is methyl, R⁴ is hydrogen, then R¹ is hydrogen;
wherein when a is zero, R¹ is hydrogen or methyl, R² is —(CH₂)_{b}—(X¹)—(Y¹)_{c}, X¹ is —CONH—, c is 1, Y¹ is an immunogenic carrier or a label, R³ is methyl, R⁴ is hydrogen, then b is 5-10;
wherein when a is zero, R² is —(CH₂)_{b}—(X¹)—(Y¹)_{c}, b is 3 or 4, X¹ is —COOH, c is zero, R³ is methyl, R⁴ is hydrogen, then R¹ is hydrogen;
wherein when a is zero, R¹ is hydrogen or methyl, R² is —(CH₂)_{b}—(X¹)—(Y¹)_{c}, X¹ is —COOH, c is zero, R³ is methyl, R⁴ is hydrogen, then b is 5-10; and
wherein at least one of R¹, R², and R³ is neither hydrogen nor an alkyl.

81. The method of claim 80, further comprising the steps of:
incubating the sample, conjugate, and antibody for a time sufficient for the antibody to bind to the MDA and/or MDMA in the sample;
adding a substrate for the enzyme to the sample; and
measuring the activity of the enzyme;
wherein the presence of the complex comprising the MDA and/or MDMA and the antibody is proportional to the activity of the enzyme.

82. (canceled)

83. (canceled)
